SEQUENCE LISTING (35 sequences)

SEQ ID NO: 1 - DNA, 1056 bp, Pelargonium x hortorum
atggcttctacctctatctctaaagttaaccacatccgtaaagttggtgttaccggtgttatggctccgcagaaaatcgaaatcttcaaatctatggaagaatggggtaaacacaacatcctgccgctggctaaaccggttgaaaaatcttggcagccgaccgacttcctgccggacccgtcttctgaaggtttcatggaagaatacaacgctttcaaagaacgtacccgtgaactgccggacgaatacttcgttgttctggctggtgacatgatcaccgaagaagctctgccgacctaccagaccctggttaaccgtccggacgaagttgctgacgaaaccggtcactctgaatctccgtgggctgtttggtctcgtgcttggaccgctgaagaaaaccgtcacggtgacctgctgaacaaatacctgtacctgtctggtaaactggacatgcgtcaggttgaaaaaaccatccagtacctgatcgctctgggtcaggacatcggtaccgaaaaaaacccgtaccacctgttcatctacacctctttccaggaacgtgctaccttcatctctcacgctaacaccgctaaactggctcagcagcacggtgacaaacagctggctcagatctgcggtaccatcgctgctgacgaaaaacgtcacgaaaccgcttacacccgtatcgttgacaaactgttcgaactggacccggacgaaaccatgtcttgcctggctcacatgatgaaacgtaaaatcaccatgccggctcacctgatgcgtgacggtcgtgacccgcacctgttccagcacttctctgtggttgcttctcgtaccggtgtttacaccgttatggactacatcaacatcctggaacacttcgttgaaaaatggaacatcgaaaaaatcaccgctggtctgtctgacaaaggtcgtgaagctcaggactacgtttgcaaactgggtgaacgtctgcgtaaagttgaagaacgtgctcaccagcgtgttgttcaggctgacccgatcccgttctcttggatcttcgaccgtaaagtttaa

SEQ ID NO: 2 - protein, 351 aa, Pelargonium x hortorum
MASTSISKVNHIRKVGVTGVMAPQKIEIFKSMEEWGKHNILPLAKPVEKSWQPTDFLPDPSSEGFMEEYNAFKERTRELPDEYFVVLAGDMITEEALPTYQTLVNRPDEVADETGHSESPWAVWSRAWTAEENRHGDLLNKYLYLSGKLDMRQVEKTIQYLIALGQDIGTEKNPYHLFIYTSFQERATFISHANTAKLAQQHGDKQLAQICGTIAADEKRHETAYTRIVDKLFELDPDETMSCLAHMMKRKITMPAHLMRDGRDPHLFQHFSVVASRTGVYTVMDYINILEHFVEKWNIEKITAGLSDKGREAQDYVCKLGERLRKVEERAHQRVVQADPIPFSWIFDRKV

SEQ ID NO: 3 - DNA, 921 bp, Nostoc punctiforme
atgactcaagcgaaagccaaaaaagaccacggtgacgttcctgttaacacttaccgtcccaatgctccatttattggcaaggtaatatctaatgaaccattagtcaaagaaggtggtattggtattgttcaacaccttaaatttgacctatctggtggggatttgaagtatatagaaggtcaaagtattggcattattccgccaggtttagacaagaacggcaagcctgaaaaactcagactatattccatcgcctcaactcgtcatggtgatgatgtagatgataagacagtatcactgtgcgtccgccagttggagtacaagcacccagaaactggcgaaacagtctacggtgtttgctctacgcacctgtgtttcctcaagccaggggaagaggtaaaaattacagggcctgtgggtaaggaaatgttgttacccaatgaccctgatgctaatgttatcatgatggctactggaacaggtattgcgccgatgcgggcttacttgtggcgtcagtttaaagatgcggaaagagcggctaacccagaataccaatttaaaggattctcttggctaatatttggcgtacctacaactccaaaccttttatataaggaagaactggaagagattcaacaaaaatatcctgagaacttccgcctaactgctgccatcagccgcgaacagaaaaatccccaaggcggtagaatgtatattcaagaccgcgtagcagaacatgctgatgaattgtggcagttgattaaaaatgaaaaaacccacacttacatttgcggtttgcgcggtatggaagaaggtattgatgcagccttaactgctgctgctgctaaggaaggcgtaacctggagtgattaccagaagcaactcaagaaagccggtcgctggcacgtagaaacttactaa

SEQ ID NO: 4 - protein, 306 aa, Nostoc punctiforme
MTQAKAKKDHGDVPVNTYRPNAPFIGKVISNEPLVKEGGIGIVQHLKFDLSGGDLKYIEGQSIGIIPPGLDKNGKPEKLRLYSIASTRHGDDVDDKTVSLCVRQLEYKHPETGETVYGVCSTHLCFLKPGEEVKITGPVGKEMLLPNDPDANVIMMATGTGIAPMRAYLWRQFKDAERAANPEYQFKGFSWLIFGVPTTPNLLYKEELEEIQQKYPENFRLTAAISREQKNPQGGRMYIQDRVAEHADELWQLIKNEKTHTYICGLRGMEEGIDAALTAAAAKEGVTWSDYQKQLKKAGRWHVETY

SEQ ID NO: 5 - DNA, 300 bp, Nostoc punctiforme
atgccaacttataaagtgacactaattaacgaggctgaagggctgaacacaacccttgatgttgaggacgatacctatattctagacgcagctgaagaagctggtattgacctgccctactcttgccgcgctggtgcttgctctacttgtgcaggtaaactcgtatcaggtaccgtcgatcaaggcgatcaatcattcttagatgacgatcaaatagaagctggatatgtactgacctgtgttgcttacccaacttctaatgtcacgatcgaaactcacaaagaagaagaactctattaa

SEQ ID NO: 6 - protein, 99 aa, Nostoc punctiforme
MPTYKVTLINEAEGLNTTLDVEDDTYILDAAEEAGIDLPYSCRAGACSTCAGKLVSGTVDQGDQSFLDDDQIEAGYVLTCVAYPTSNVTIETHKEEELY

SEQ ID NO: 7 - DNA, 939 bp, Arabidopsis thaliana
atggctgttgtatctgctgatcaaggtagtgtggttcaaggtttggctactctcgcggatcagctccgattaggtagtttgactgaagatggtttatcttataaagagaagtttgttgttagatcttacgaagtgggtagtaacaaaaccgctactgttgaaaccattgctaatcttttacaggaggtgggatgtaatcatgcacaaagtgttggtttttcgactgatgggtttgcaacaacaactactatgaggaagttgcatctcatttgggttactgcgagaatgcatatcgagatctataagtaccctgcttggggtgatgtggttgagatagagacttggtgtcagagtgaaggaaggattgggacaaggcgtgattggattcttaaggattctgtcactggtgaagtcactggccgtgctacaagcaagtgggtgatgatgaaccaagacacgagacggcttcagaaagtttctgatgatgttcgggacgagtacttggtcttctgtcctcaagaaccgaggttagcatttccggaagagaataaccgtagcttgaagaaaatcccgaaactcgaagatccggctcagtattcaatgattgggcttaagcctagacgagctgatctcgacatgaaccagcatgtcaataatgtcacctatattggatgggttctggagagcataccacaagaaattgtagacacgcacgagcttcaggtcataactctggattatagaagagaatgtcaacaagacgatgtggtggattcactcaccaccaccacctctgaaattggtggaaccaatggctctgccacgtctggcacacagggccacaacgatagccagttcttgcacctcctgaggttgtctggagatggtcaggagatcaaccgcgggacaactctgtggagaaagaagccttcaagttaa

SEQ ID NO: 8 - protein, 312 aa, Arabidopsis thaliana
MAVVSADQGSVVQGLATLADQLRLGSLTEDGLSYKEKFVVRSYEVGSNKTATVETIANLLQEVGCNHAQSVGFSTDGFATTTTMRKLHLIWVTARMHIEIYKYPAWGDVVEIETWCQSEGRIGTRRDWILKDSVTGEVTGRATSKWVMMNQDTRRLQKVSDDVRDEYLVFCPQEPRLAFPEENNRSLKKIPKLEDPAQYSMIGLKPRRADLDMNQHVNNVTYIGWVLESIPQEIVDTHELQVITLDYRRECQQDDVVDSLTTTTSEIGGTNGSATSGTQGHNDSQFLHLLRLSGDGQEINRGTTLWRKKPSS

SEQ ID NO: 9 - DNA, 762 bp, Clostridium thermocellum
atgcagaagaaaagattttcaaagaagtatgaagtacattactacgaaatcaactcaatgcaggaagcaactcttctctccctgctaaactatatggaggactgcgcaatatcccactcaacctctgccggatacggtgtcaacgagttattggctgctgacgcaggatgggtattataccgctggttaattaaaatagacagacttcccaagctcggagaaacaattactgttcagacatgggcctcttccttcgaacgcttctacggcaacagggaatttatcgtattggacggcagggataaccccattgtcaaagcctcatccgtatggatatatttcaatattaaaaaaagaaaacctatgagaatccccctcgaaatgggagatgcttatggcatagacgaaacaagagctttggaagaaccctttaccgacttcgattttgattttgaacccaaagttattgaagaatttactgtaaaaagaagtgatatagacacaaacagccacgtaaacaacaagaaatacgttgactggattatggaaaccgtaccccagcaaatatatgacaactacaaagttacatctcttcagattatatacaaaaaggaatcttctttgggttcaggcataaaggccggatgtgtaattgatgagcaaaataccgataatccgcggctccttcacaaaatatgggacaagaataccggtttggagcttgtatccgccgaaacaatctggcaaaagattcagtcataa

SEQ ID NO: 10 - protein, 240 aa, Hungateiclostridium thermocellum
MQKKRFSKKYEVHYYEINSMQEATLLSLLNYMEDCAISHSTSAGYGVNELLAADAGWVLYRWLIKIDRLPKLGETITVQTWASSFERFYGNREFIVLDGRDNPIVKASSVWIYFNIKKRKPMRIPLEMGDAYGIDETRALEEPFTDFDFDFEPKVIEEFTVKRSDIDTNSHVNNKKYVDWIMETVPQQIYDNYKVTSLQIIYKKESSLGSGIKAGCVIDEQNTDNPRLLHKIWDKNTGLE

SEQ ID NO: 11 - DNA, 1089 bp, Vitis vinifera
atggcttccatgcaccgctctgtgtccagggagattaagaatacaaagaagacttctagctctcctcgcaaggtgcaagtaacccattcaatgccaccacacaagattgagattttcaaatccatggagaattgggttgaggagaacgttttaattcacctgaagccagttgagaaatgttggcaacctcaggattttctgcctcatcctgcttctgatggatttcatgagcgagtcgaggagctaaaggagagagcaaaggggatcccggatgactactttgtcgttttggttggagatatgatcactgaagaagcccttccaacttaccaaacactgttcaataccactgatggaatccgtgatgaaacaggtgcaagccccacttcttgggcaacttggacaagggcatggaccgctgaagagaacaggcacggtgaccttcttaataagtatctctatctgtctggaagagtagacatgaaacaaattgagaagacgatccagtatttgattagggctggaatggatttccagacggaaaacaatccgtaccttttattcatctatacttcatttcaagaaagggcaaccttcatatcccatggcaatactgccaggctcgccaagcaacatggggacaagagcttggctcaaatatgtggcataatagcctcagatgagaagcgccatgaaactgcctacaccaagatagtggaaaagctctttgagattgatcccaatgggactgtcttggcttttgcagacaggatgaggaagaaaatcaccatgccggccctcttgatgtatgatggatgtgatgacgacctttttgaacacttctcagcagttgctcagcggcttggtgtgtatactgccaaggactatgttgataacttagaattctttgtggaaagatggaatgtggaaaagctaactgggctttctagtgaggggcgaaaagctcaggattatgtttgtgggttagctaaaagattgagaacactggaggagagagctcaagaaaaggctaagcaagcacccaccattcctttcagttggatttttgatagagaagtgaagctctga

SEQ ID NO: 12 - protein, 360 aa, Vitis vinifera
MASMHRSVSREIKNTKKTSSSPRKVQVTHSMPPHKIEIFKSMENWVEENVLIHLKPVEKCWQPQDFLPHPASDGFHERVEELKERAKGIPDDYFVVLVGDMITEEALPTYQTLFNTTDGIRDETGASPTSWATWTRAWTAEENRHGDLLNKYLYLSGRVDMKQIEKTIQYLIRAGMDFQTENNPYLLFIYTSFQERATFISHGNTARLAKQHGDKSLAQICGIIASDEKRHETAYTKIVEKLFEIDPNGTVLAFADRMRKKITMPALLMYDGCDDDLFEHFSAVAQRLGVYTAKDYVDNLEFFVERWNVEKLTGLSSEGRKAQDYVCGLAKRLRTLEERAQEKAKQAPTIPFSWIFDREV

SEQ ID NO: 13 - DNA, 747 bp, Escherichia coli
atggctgattgggtaacaggcaaagtcactaaagtgcagaactggaccgacgccctgtttagtctcaccgttcacgcccccgtgcttccgtttaccgccgggcaatttaccaagcttggccttgaaatcgacggcgaacgcgtccagcgcgcctactcctatgtaaactcgcccgataatcccgatctggagttttacctggtcaccgtccccgatggcaaattaagcccacgactggcggcactgaaaccaggcgatgaagtgcaggtggttagcgaagcggcaggattctttgtgctcgatgaagtgccgcactgcgaaacgctatggatgctggcaaccggtacagcgattggcccttatttatcgattctgcaactaggtaaagatttagatcgcttcaaaaatctggtcctggtgcacgccgcacgttatgccgccgacttaagctatttgccactgatgcaggaactggaaaaacgctacgaaggaaaactgcgcattcagacggtggtcagtcgggaaacggcagcggggtcgctcaccggacggataccggcattaattgaaagtggggaactggaaagcacgattggcctgccgatgaataaagaaaccagccatgtgatgctgtgcggcaatccacagatggtgcgcgatacacaacagttgctgaaagagacccggcagatgacgaaacatttacgtcgccgaccgggccatatgacagcggagcattactggtaa

SEQ ID NO: 14 - protein, 247 aa, Escherichia coli
MADWVTGKVTKVQNWTDALFSLTVHAPVLPFTAGQFTKLGLEIDGERVQRAYSYVNSPDNPDLEFYLVTVPDGKLSPRLAALKPGDEVQVVSEAAGFFVLDEVPHCETLWMLATGTAIGPYLSILQLGKDLDRFKNLVLVHAARYAADLSYLPLMQELEKRYEGKLRIQTVVSRETAAGSLTGRIPALIESGELESTIGLPMNKETSHVMLCGNPQMVRDTQQLLKETRQMTKHLRRRPGHMTAEHY

SEQ ID NO: 15 - DNA, 488 bp, Acinetobacter baylyi
atgatgactgagtcaaacactcctgcgtttacgatccctgaattgccaatggacattcaaaaaattcgtgaatatttgccacatcgctatccattcctattggttgatcgtgtagttgaagttggtgaaaataatattgttggctataaaaacgtttcgattaacgaagagttttttcagggccattttcctgattatccgattatgccaggtgtcttgattgtagaggctttggcacaaatttctggtattttgggttttattatgaataatgaaactcctaaaccaggttctttgtttctgttcgcaggggctgagaaagttcgtttcaaaaaacaagtggttgctggtgatcagctcgttttaaaagctgaacttgtcatgcaaaaacgtggtatctacaaatacaattgtactgctacggttgatggtaaagtcgctacaaccgctgaaattatagtttcgcatttaagaacagagcaggcatg

SEQ ID NO: 16 - protein, 162 aa, Acinetobacter baylyi
MMTESNTPAFTIPELPMDIQKIREYLPHRYPFLLVDRVVEVGENNIVGYKNVSINEEFFQGHFPDYPIMPGVLIVEALAQISGILGFIMNNETPKPGSLFLFAGAEKVRFKKQVVAGDQLVLKAELVMQKRGIYKYNCTATVDGKVATTAEIIVSHLRTEQA

SEQ ID NO: 17 - DNA, 3525 bp, Mycobacterium smegmatis
atgggcacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttag

SEQ ID NO: 18 - protein, 1174 aa, Mycobacterium smegmatis
MGTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 19 - DNA, 1026 bp, Acinetobacter baylyi
atggcaacaactaatgtgattcatgcttatgctgcaatgcaggcaggtgaagcactcgtgccttattcgtttgatgcaggcgaactgcaaccacatcaggttgaagttaaagtcgaatattgtgggctgtgccattccgatgtctcggtactcaacaacgaatggcattcttcggtttatccagtcgtggcaggtcatgaagtgattggtacgattacccaactgggaagtgaagccaaaggactaaaaattggtcaacgtgttggtattggctggacggcagaaagctgtcaggcctgtgaccaatgcatcagtggtcagcaggtattgtgcacgggcgaaaataccgcaactattattggtcatgctggtggctttgcagataaggttcgtgcaggctggcaatgggtcattcccctgcccgacgaactcgatccgaccagtgctggtcctttgctgtgtggcggaatcacagtatttgatccaattttaaaacatcagattcaggctattcatcatgttgctgtgattggtatcggtggtttgggacatatggccatcaagctacttaaagcatggggctgtgaaattactgcgtttagttcaaatccaaacaaaaccgatgagctcaaagctatgggggccgatcacgtggtcaatagccgtgatgatgccgaaattaaatcgcaacagggtaaatttgatttactgctgagtacagttaatgtgcctttaaactggaatgcgtatctaaacacactggcacccaatggcactttccattttttgggcgtggtgatggaaccaatccctgtacctgtcggtgcgctgctaggaggtgccaaatcgctaacagcatcaccaactggctcgcctgctgccttacgtaagctgctcgaatttgcggcacgtaagaatatcgcacctcaaatcgagatgtatcctatgtcggagctgaatgaggccatcgaacgcttacattcgggtcaagcacgttatcggattgtacttaaagccgatttttaa

SEQ ID NO: 20 - protein, 341 aa, Acinetobacter baylyi
MATTNVIHAYAAMQAGEALVPYSFDAGELQPHQVEVKVEYCGLCHSDVSVLNNEWHSSVYPVVAGHEVIGTITQLGSEAKGLKIGQRVGIGWTAESCQACDQCISGQQVLCTGENTATIIGHAGGFADKVRAGWQWVIPLPDELDPTSAGPLLCGGITVFDPILKHQIQAIHHVAVIGIGGLGHMAIKLLKAWGCEITAFSSNPNKTDELKAMGADHVVNSRDDAEIKSQQGKFDLLLSTVNVPLNWNAYLNTLAPNGTFHFLGVVMEPIPVPVGALLGGAKSLTASPTGSPAALRKLLEFAARKNIAPQIEMYPMSELNEAIERLHSGQARYRIVLKADF

SEQ ID NO: 21 - DNA, 2400 bp, Artificial Sequence
  Hybrid fusion cytochrome P450 Cyp153A16 (G307A)
atgccaacactgcccagaacatttgacgacattcagtcccgactgattaacgccacctccagggtggtgccgatgcagaggcaaattcagggactgaaattcttaatgagcgccaagaggaagaccttcggcccacgccgaccgatgcccgaattcgttgaaacacccatcccggacgttaacacgctggcccttgaggacatcgatgtcagcaatccgtttttataccggcagggtcagtggcgcgcctatttcaaacggttgcgtgatgaggcgccggtccattaccagaagaacagccctttcggccccttctggtcggtaactcggtttgaagacatcctgttcgtggataagagtcacgacctgttttccgccgagccgcaaatcattctcggtgaccctccggaggggctgtcggtggaaatgttcatagcgatggatccgccgaaacacgatgtgcagcgcagctcggtgcagggagtagtggcaccgaaaaacctgaaggagatggaggggctgatccgatcacgcaccggcgatgtgcttgacagcctgcctacagacaaaccctttaactgggtacctgctgtttccaaggaactcacaggccgcatgctggcgacgcttctggattttccttacgaggaacgccacaagctggttgagtggtcggacagaatggcaggtgcagcatcggccaccggcggggagtttgccgatgaaaatgccatgtttgacgacgcggcagacatggcccggtctttctccaggctttggcgggacaaggaggcgcgccgcgcagcaggcgaggagcccggtttcgatttgatcagcctgttgcagagcaacaaagaaacgaaagacctgatcaatcggccgatggagtttatcggtaatttgacgctgctcatagtcgccggcaacgatacgacgcgcaactcgatgagtggtggcctggtggccatgaacgaattccccagggaatttgaaaaattgaaggcaaaaccggagttgattccgaacatggtgtcggaaatcatccgctggcaaacgccgctggcctatatgcgccgaatcgccaagcaggatgtcgaactgggcggccagaccatcaagaagggtgatcgagttgtcatgtggtacgcgtcgggtaaccgggacgagcgcaaatttgacaaccccgatcagttcatcattgatcgcaaggacgcacgaaaccacatgtcgttcggctatggggttcaccgttgcatgggcaaccgtctggctgaactgcaactgcgcatcctctgggaagaaatactcaagcgttttgacaacatcgaagtcgtcgaagagcccgagcgggtgcagtccaacttcgtgcggggctattccaggttgatggtcaaactgacaccgaacagtgtactccatcgtcatcaacctgtcaccatcggcgagccggccgctcgtgctgtgagccgcacggtgaccgttgagcgtcttgatcgcattgccgacgatgtccttcgcctggtccttcgcgatgctggaggtaaaaccctcccgacgtggacgcctggcgctcacatcgacctggatctgggtgctctgagccgtcagtattcgctctgcggcgctccggatgctccgtcgtacgaaatcgccgtgcacttagatccggaaagccgtggtggaagccgctatattcatgaacagctggaagttggaagtccgctgcgtatgcgtggcccacgcaaccatttcgccctggatccgggtgcggaacattacgtgtttgttgccgggggtatcggcatcacgccggtgctggcaatggcggatcatgcccgtgcgcgtggttggtcgtacgaactgcattattgtggtcgtaatcgtagcggtatggcttacctggaacgcgtcgcgggacatggtgaccgcgctgccttgcacgtatctgaagaaggcacccgcattgatctggcggcattacttgctgaaccggcgccgggcgtgcaaatctacgcctgcggtccgggccgtttattagcgggtcttgaagacgcgtctcgtaattggccggatggcgcgcttcatgtggagcatttcacttcgagtttagccgctttggatccggatgtcgaacatgcctttgatttggagctgcgtgactctggccttaccgttcgcgtcgagccaactcagaccgttttagacgctttgcgtgcgaacaatatcgacgtcccgtcggattgcgaagaggggctgtgtggttcttgcgaagtagccgttctggatggcgaggttgatcaccgtgataccgttctgactaaggccgagcgcgccgcgaatcgtcagatgatgacttgctgcagtcgtgcatgcggtgatcgtctggcgctgcgcctctaa

SEQ ID NO: 22 - protein, 799 aa, Artificial Sequence
  Hybrid fusion cytochrome P450 Cyp153A16(G307A)
MPTLPRTFDDIQSRLINATSRVVPMQRQIQGLKFLMSAKRKTFGPRRPMPEFVETPIPDVNTLALEDIDVSNPFLYRQGQWRAYFKRLRDEAPVHYQKNSPFGPFWSVTRFEDILFVDKSHDLFSAEPQIILGDPPEGLSVEMFIAMDPPKHDVQRSSVQGVVAPKNLKEMEGLIRSRTGDVLDSLPTDKPFNWVPAVSKELTGRMLATLLDFPYEERHKLVEWSDRMAGAASATGGEFADENAMFDDAADMARSFSRLWRDKEARRAAGEEPGFDLISLLQSNKETKDLINRPMEFIGNLTLLIVAGNDTTRNSMSGGLVAMNEFPREFEKLKAKPELIPNMVSEIIRWQTPLAYMRRIAKQDVELGGQTIKKGDRVVMWYASGNRDERKFDNPDQFIIDRKDARNHMSFGYGVHRCMGNRLAELQLRILWEEILKRFDNIEVVEEPERVQSNFVRGYSRLMVKLTPNSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGPGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 23 - DNA, 1578 bp, Saccharomyces cerevisiae
atgaacgaaatcgacgaaaaaaaccaggctccggttcaccaggaatgcctgaaagaaatgatccagaacggtcacgctcgtcgtatgggttctgttgaagacctgtacgttgctctgaaccgtcagaacctgtaccgtaacttctgcacctacggtgaactgtctgactactgcacccgtgaccagctgaccctggctctgcgtgaaatctgcctgaaaaacccgaccctgctgcacatcgttctgccgacccgttggccgaaccacgaaaactactaccgttcttctgaatactactctcgtccgcacccggttcacgactacatctctgttctgcaggaactgaaactgtctggtgttgttctgaacgaacagccggaatactctgctgttatgaaacagatcctggaagaattcaaaaactctaaaggttcttacaccgctaaaatcttcaaactgaccaccaccctgaccatcccgtacttcggtccgaccggtccgtcttggcgtctgatctgcctgccggaagaacacaccgaaaaatggaaaaaattcatcttcgtttctaaccactgcatgtctgacggtcgttcttctatccacttcttccacgacctgcgtgacgaactgaacaacatcaaaaccccgccgaaaaaactggactacatcttcaaatacgaagaagactaccagctgctgcgtaaactgccggaaccgatcgaaaaagttatcgacttccgtccgccgtacctgttcatcccgaaatctctgctgtctggtttcatctacaaccacctgcgtttctcttctaaaggtgtttgcatgcgtatggacgacgttgaaaaaaccgacgacgttgttaccgaaatcatcaacatctctccgaccgaattccaggctatcaaagctaacatcaaatctaacatccagggtaaatgcaccatcaccccgttcctgcacgtttgctggttcgtttctctgcacaaatggggtaaattcttcaaaccgctgaacttcgaatggctgaccgacatcttcatcccggctgactgccgttctcagctgccggacgacgacgaaatgcgtcagatgtaccgttacggtgctaacgttggtttcatcgacttcaccccgtggatctctgaattcgacatgaacgacaacaaagaaaacttctggccgctgatcgaacactaccacgaagttatctctgaagctctgcgtaacaaaaaacacctgcacggtctgggtttcaacatccagggtttcgttcagaaatacgttaacatcgacaaagttatgtgcgaccgtgctatcggtaaacgtcgtggtggtaccctgctgtctaacgttggtctgttcaaccagctggaagaaccggacgctaaatactctatctgcgacctggctttcggtcagttccagggttcttggcaccaggctttctctctgggtgtttgctctaccaacgttaaaggtatgaacatcgttgttgcttctaccaaaaacgttgttggttctcaggaatctctggaagaactgtgctctatctacaaagctctgctgctgggtccgtag

SEQ ID NO: 24 - protein, 525 aa, Saccharomyces cerevisiae
MNEIDEKNQAPVHQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 25 - DNA, 621 bp, Escherichia coli
atgaaaactacgcatacctccctcccctttgccggacatacgctgcattttgttgagttcgatccggcgaatttttgtgagcaggatttactctggctgccgcactacgcacaactgcaacacgctggacgtaaacgtaaaacagagcatttagccggacggatcgctgctgtttatgctttgcgggaatatggctataaatgtgtgcccgcaatcggcgagctacgccaacctgtctggcctgcggaggtatacggcagtattagccactgtgggactacggcattagccgtggtatctcgtcaaccgattggcattgatatagaagaaattttttctgtacaaaccgcaagagaattgacagacaacattattacaccagcggaacacgagcgactcgcagactgcggtttagccttttctctggcgctgacactggcattttccgccaaagagagcgcatttaaggcaagtgagatccaaactgatgcaggttttctggactatcagataattagctggaataaacagcaggtcatcattcatcgtgagaatgagatgtttgctgtgcactggcagataaaagaaaagatagtcataacgctgtgccaacacgattaa

SEQ ID NO: 26 - protein, 206 aa, Escherichia coli
MKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 27 - protein, 141 aa, Clostridium acetobutylicum
MSLSIEQIMEIIPHRYPMLLVDRVEEIEPGKRAVGYKNVTFNEQIFQGHYPGKPIMPGVLMIEALAQLGGVAILSLDKYKGKKPILGAVKNAKFRRMVVPGDVLKLEIEIVKVKGPAGIGKGIATVNGEKAVEAEITFMIV

SEQ ID NO: 28 - protein, 155 aa, Synechococcus elongatus
MTVNPDAPALPTLPLAVETIQGLLPHRYPFALVDRIIDYVPGERAVGIKNVTFNEPQFQGHFPGRPLMPGVLIVEAMAQVGGVIVTLMPDMPQGLFVFAGIDQVRFRRPVVPGDQLVLSAQLLSVKRRRFCKIQGEAMVDGQLAASGELLFSLVE

SEQ ID NO: 29 - protein, 558 aa, Pseudomonas oleovorans
MYDYIIVGAGSAGCVLANRLSADPSKRVCLLEAGPRDTNPLIHMPLGIALLSNSKKLNWAFQTAPQQNLNGRSLFWPRGKTLGGSSSINAMVYIRGHEDDYHAWEQAAGRYWGWYRALELFKRLECNQRFDKSEHHGVDGELAVSDLKYINPLSKAFVQAGMEANINFNGDFNGEYQDGVGFYQVTQKNGQRWSSARAFLHGVLSRPNLDIITDAHASKILFEDRKAVGVSYIKKNMHHQVKTTSGGEVLLSLGAVGTPHLLMLSGVGAAAELKEHGVSLVHDLPEVGKNLQDHLDITLMCAANSREPIGVALSFIPRGVSGLFSYVFKREGFLTSNVAESGGFVKSSPDRDRPNLQFHFLPTYLKDHGRKIAGGYGYTLHICDLLPKSRGRIGLKSANPLQPPLIDPNYLSDHEDIKTMIAGIKIGRAILQAPSMAKHFKHEVVPGQAVKTDDEIIEDIRRRAETIYHPVGTCRMGKDPASVVDPCLKIRGLANIRVVDASIMPHLVAGNTNAPTIMIAENAAEIIMRNLDVEALEASAEFAREGAELELAMIAVCM

SEQ ID NO: 30 - protein, 503 aa, Acinetobacter baylyi
MRYIDPNQPGSKVQFKAQYENFIGGQWVPPVKGEYFGNSSPVDGKVFTQIPRSSVEDIELALDAAHKAKADWNKASPTVRSNVLLKIADRLEENLELLAVAETWENGKPIRETLAADIPLAIDHFRYFAGCIRAQEGGISEIDEDTIAYHFHEPLGVVGQIIPWNFPILMAAWKLAPALAAGNCIVLKPAEQTPSSILVLAELIQDLLPPGVLNIVNGYGAEVGRPLATNPRISKIAFTGSTKVGQMIMQYATENIIPVTLELGGKSPNIFFEDILDKEDDYLEKTLEGFAMFALNQGEVCTCPSRALVQESIADKFLEMAVERVKRIKTGHPLDTETMIGAQASKQQFDKILGCIDTGRNEGAQLLTGGDARHDVDGGFYIEPTIFKGNNSMKIFQEEIFGPVLSVTTFKDFDDAMRIANDTIYGLGAGVWSRSAHTSYRAGRAIEAGRVWTNCYHLYPAHAAFGGYKQSGIGRENHRMMLDHYQQTKNLLVSYSTKPMGFF

SEQ ID NO: 31 - protein, 506 aa, Marinobacter aquaeoli
MIYAQPGQEGSVVSFKSRYENYIGGEWVAPVKGQYFDNITPVTGAVFCEVPRSTAEDIDLALDAAHKAAPAWGKTSPTERSNILLKIADRIEANLEKLAVAETWDNGKAVRETLNADVPLAADHLRYFAGCIRAQEGHMSEIDHNTVAYHFHEPLGVVGQIIPWNFPLLMAAWKLGPCLASGNCTVLKPAEQTPASILVLMDIIGDLLPPGVINIVNGYGIEAGQALATSKRIAKIAFTGSTPVGSHILKCAAENIIPSTVELGGKSPNIYFSDVMKAEPEFVDKCVEGLVLAFFNQGEICTCPSRALVQEDMFEEFMQKVVERTKSIKRGNPLDTDVQVGAQASKEQFDKIMSYMEIGRQEGAVVLTGGDREHLEGEFNNGFYIQPTLFKGDNKMRVFQEEIFGPVVGVTTFKTEEEALAIANDTEFGLGAGVWTRDTNLAYRMGRNIQAGRVWMNCYHAYPAHAAFGGYKKSGIGRENHKMALEHYQQTKCMLTSYDTNPLGFF

SEQ ID NO: 32 - protein, 483 aa, Pseudomonas oleovorans
MTIPISLAKLNSSADTHSALEVFNLQKVASSARRGKFGIAERIAALNLLKETIQRREPEIIAALAADFRKPASEVKLTEIFPVLQEINHAKRNLKDWMKPRRVRAALSVAGTRAGLRYEPKGVCLIIAPWNYPFNLSFGPLVSALAAGNSVVIKPSELTPHTATLIGSIVREAFSVDLVAVVEGDAAVSQELLALPFDHIFFTGSPRVGKLVMEAASKTLASVTLELGGKSPTIIGPTANLPKAARNIVWGKFSNNGQTCIAPDHVFVHRCIAQKFNEILVKEIVRVYGKDFAAQRRSADYCRIVNDQHFNRINKLLTDAKAKGAKILQGGQVDATERLVVPTVLSNVTAAMDINHEEIFGPLLPIIEYDDIDSVIKRVNDGDKPLALYVFSEDKQFVNNIVARTSSGSVGVNLSVVHFLHPNLPFGGVNNSGIGSAHGVYGFRAFSHEKPVLIDKFSITHWLFPPYTKKVKQLIGITVKYLS

SEQ ID NO: 33 - protein, 406 aa, Escherichia coli
MKRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKAMTGHSLGAAGVQEAIYSLLMLEHGFIAPSINIEELDEQAAGLNIVTETTDRELTTVMSNSFGFGGTNATLVMRKLKD

SEQ ID NO: 34 - DNA, 1026 bp, Synechococcus elongatus
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 35 - protein, 341 aa, Synechococcus elongatus
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

(19) United States
(12) Patent Application Publication
(10) Pub. No.: US 2024/0229086 A1
(43) Pub. Date: Jul. 11, 2024

(54) Production Of Non-Native Monounsaturated Fatty Acids In Bacteria

(71) Applicant: GENOMATICA, INC., San Diego, CA (US)

(72) Inventors: Andreas W. SCHIRMER, San Diego, CA (US); Erin Frances PERRY, San Marcos, CA (US); Alma Itzel RAMOS-SOLIS, San Diego, CA (US); Angelica ZABALA-BAUTISTA, San Diego, CA (US)

(21) Appl. No.: 18/284,349

(22) PCT Filed: Mar. 22, 2022
(86) PCT No.: PCT/US2022/021290

§ 371 Date: Sep. 27, 2023

Related U.S. Application Data
(60) Provisional application No. 63/167,355, filed on Mar. 29, 2021.

(51) Int. Cl. C12P 7/6409; C12N 1/20; C12N 9/02; C12N 9/04; C12N 9/10; C12N 9/12; C12N 9/16; C12N 9/88

(52) U.S. Cl. CPC C12P 7/6409; C12N 1/20; C12N 9/0006; C12N 9/0008; C12N 9/0071; C12N 9/1029; C12N 9/1288; C12N 9/16; C12N 9/88; C12Y 114/19002; C12Y 203/01084; C12Y 207/08007; C12Y 301/02014; C12Y 402/01059

(57) ABSTRACT

Recombinant proteobacteria, including γ-proteobacteria, comprising a heterologous acyl-ACP desaturase and a heterologous acyl-ACP thioesterase, wherein the native dual 3-hydroxy acyl-ACP dehydratase/isomerase is deleted are provided herein. The recombinant proteobacteria produce non-native monounsaturated free fatty acids or derivatives thereof. Methods of producing non-native monounsaturated free fatty acids or derivatives thereof are also provided, in addition to cell cultures and fatty acid compositions produced by the recombinant proteobacteria. The recombinant proteobacteria may be used to produce insect pheromones or precursors thereof, and fragrances or precursors thereof.

### REFERENCE TO A SEQUENCE LISTING

This application contains references to amino acid sequences and/or nucleic acid sequences which have been submitted concurrently herewith as the sequence listing text file entitled “6281099_1.TXT”, file size 93 KiloBytes (KB), created on 14 Mar. 2022. The aforementioned sequence listing is hereby incorporated by reference in its entirety pursuant to 37 C.F.R. § 1.52(e)(5).

### FIELD

The disclosure relates to the field of specialty chemicals and methods for their synthesis. The disclosure provides proteobacteria engineered to produce non-native monounsaturated fatty acids and derivatives thereof. The disclosure further provides biochemical pathways, recombinant microorganisms and methods for the biological production of various monounsaturated free fatty acids and derivatives thereof.

### BACKGROUND

Monounsaturated fatty acids (mUFAs) and fatty acid derivatives are attractive as the basis for many different products. For example, mUFAs are a component of good nutrition (see e.g., Nettleton J. A (2016) Ann Nutr Metab. 68:249-257); they serve as the basis for production of numerous useful molecules such as e.g., flavors and fragrances (see e.g., International Patent Application Publication WO 2016/157719). Production of musk fragrance precursors from native mUFA derivatives (e.g., musk macrolactone cis-isoambrettolide) or from non-native ω-9 mUFA derivatives (e.g., musk macrolactone ambrettolide) have been described. See WO 2014/201474 and WO 2020/047088, both incorporated herein by reference in their entirety.

Additionally, mUFAs are also ideal components for biodiesel since mUFAs improve fluidity at low temperatures and contribute to oxidative stability of the biodiesel product (see e.g., Yujin Cao et al. (2014) Biotechnol Biofuels.; 7: 59).

Most sources of mUFAs for nutrition, biodiesel, and for flavor and/or fragrance chemicals depend on plant or animal origins and thus can be limited in both quantity and quality.

In recent years, technology for the production of fatty acids and fatty acid derivatives has been successfully developed see e.g., U.S. Pat. Nos. 9,951,322; 9,890,401; 9,879,239; 9,873,865; 9,758,769; 9,683,247; 9,683,219; 9,670,512; 9,598,706; 9,587,231; 9,481,899 (each of which is incorporated herein by reference in its entirety). It would be greatly beneficial to be able to use such technology for the industrial scale production of mUFAs and fatty acid derivatives. In particular, it would be greatly beneficial to use recombinant proteobacteria to prepare mUFAs and fatty acid derivatives.

Recombinant proteobacteria possess many advantages over other microorganisms for the industrial production of fatty acid derivatives (see e.g., Front Microbiol. 2014; 5: 172.). Despite the advantages, there are drawbacks when it comes to the production of mUFAs and fatty acid derivatives. As is well known in the art, most proteobacteria, such as E. coli, incorporate double bonds into mUFAs between the seventh and eighth carbon counting from the reduced end of the carbon chain (i.e., at the omega-7 (ω-7) position). Thus, the production of monounsaturated fatty acids having double bonds in e.g., the ω-3, ω-5, ω-6, ω-8, ω-9, ω-11, ω-12, etc. positions are non-native to most proteobacteria.

Most proteobacteria incorporate double bonds in the ω-7 position of mUFAs through the paradigmatic “oxygen independent” (anaerobic) mUFA biosynthetic pathway (see e.g., Magnuson et al. (1993) Appl. Microbial Rev. 57(3):522-542). E. coli has two 3-hydroxy acyl-ACP dehydratases, FabZ and FabA. The key enzyme for mUFA synthesis is FabA, which is a dual function dehydratase/isomerase. FabA isomerizes trans-2-decenoyl-ACP to cis-3-decenoyl-ACP. The latter cannot be reduced by trans-2-enoyl-ACP reductase (FabI), but can be elongated by R-keto-acyl-ACP synthase I (FabB) thereby fixing the double bond position in the ω-7 position (see e.g., FIG. 1). FabA is very specific for isomerizing trans-2-decenoyl-ACP leading to the predominant production of ω-7 mUFAs in these proteobacteria.

Therefore, with regard to the synthesis of diverse mUFAs, manufacturers who desire to produce mUFAs having a double bond in a position other than the ω-7 position and who desire to harness the advantages and power of a bacterial platform are faced with a conundrum. Proteobacteria, which can produce mUFAs on an industrial scale, generally produce mUFAs with a double bond in the ω-7 position of the fatty acid. Thus, wild-type proteobacteria are limited in the breadth of mUFAs they can produce.

Consequently, new methods are needed for the production of mUFA molecules that allow for the production of a full spectrum of mUFA molecules, including, but not limited to, mUFAs with double bonds in non-native positions, such as ω-3, ω-5, ω-6, ω-8, ω-9, ω-11, ω-12, etc.

### SUMMARY

Disclosed herein are recombinant proteobacteria comprising an acyl-ACP desaturase and an acyl-ACP thioesterase, one or both which may be heterologous, wherein a native dual 3-hydroxy acyl-ACP dehydratase/isomerase (FabA) is deleted or attenuated, and wherein the recombinant proteobacterium produces a non-native mUFA or a derivative thereof. In various embodiments, the recombinant proteobacteria further comprises one or more additional enzymes such as a ferredoxin, a ferredoxin reductase, and/or a flavodoxin reductase, a 3-hydroxyacyl-ACP-dehydratase (FabZ), a carboxylic acid reductase, an alcohol dehydrogenase, a phosphopantetheinyl transferase, an alcohol acetyl-CoA transferase, a ω-hydroxylase, an alcohol oxidase/dehydrogenase, a fatty acid metabolism regulator protein (fadR), a aldehyde hydrogenase, and a β-ketoacyl-ACP synthase, wherein one or more of the additional enzymes are heterologous. In some recombinant proteobacteria comprising an acyl-ACP desaturase and an acyl-ACP thioesterase, the recombinant proteobacteria further comprise a heterologous dual 3-hydroxy acyl-ACP dehydratase/isomerase (FabA).

The recombinant proteobacterium may be a γ-proteobacterium, such as Escherichia coli. The recombinant proteobacterium may produce one or more non-native mUFAs, including, but not limited to, mUFAs having a double bond at the ω-3, ω-5, ω-6, ω-9, ω-11, ω-12, or other position.

Also disclosed herein are methods for producing a non-native mUFA or a derivative thereof, said method comprising culturing a recombinant proteobacterium comprising a heterologous acyl-ACP desaturase and a heterologous acyl-ACP thioesterase, wherein the native dual 3-hydroxy acyl-ACP dehydratase/isomerase (FabA) is deleted or attenuated.

Also disclosed herein are nucleotide sequences encoding one or both of a heterologous acyl-ACP desaturase and/or a heterologous acyl-ACP thioesterase, which is operably linked to one or more heterologous regulatory elements. The nucleotide sequence may be in a vector.

Also disclosed herein are insect pheromones and insect pheromone precursors comprising the non-native mUFAs or derivatives thereof produced by the disclosed recombinant proteobacteria comprising a heterologous acyl-ACP desaturase and a heterologous acyl-ACP thioesterase, wherein a native dual 3-hydroxy acyl-ACP dehydratase/isomerase (FabA) is deleted or attenuated and a method for preparing the insect pheromones or insect pheromone precursors.

Also disclosed herein are fragrances and fragrance precursors comprising the non-native mUFAs or derivatives thereof produced by the disclosed recombinant proteobacteria comprising a heterologous acyl-ACP desaturase and a heterologous acyl-ACP thioesterase, wherein a native dual 3-hydroxy acyl-ACP dehydratase/isomerase (FabA) is deleted or attenuated and a method for preparing the fragrances or fragrance precursors.

Also disclosed herein is the use of the recombinant proteobacterium comprising a heterologous acyl-ACP desaturase and a heterologous acyl-ACP thioesterase, wherein a native dual 3-hydroxy acyl-ACP dehydratase/isomerase (FabA) is deleted or attenuated for producing an insect pheromone, insect pheromone precursor, fragrance, and/or fragrance precursor.

Also disclosed herein are compositions comprising more ω-5 unsaturated fatty acid derivatives than ω-7 unsaturated fatty acid derivatives. The compositions may be produced by the recombinant proteobacterium disclosed herein. For example, a composition may comprise more ω-5 unsaturated fatty alcohols than ω-7 unsaturated fatty alcohols, e.g. about 90% of ω-5 unsaturated and about 10% ω-7 unsaturated fatty alcohols (e.g. about 80% of z11-hexadecenol and about 10% of z13-octadecenol and about 10% of z9-hexadecenol).

### BRIEF DESCRIPTION OF THE DRAWINGS

FIG. 1 depicts the bacterial biosynthetic pathway to convert acyl-ACPs to pheromones or pheromone precursors.

FIG. 2 depicts the bacterial biosynthetic pathway to convert acyl-ACPs to fragrances or fragrance precursors.

FIG. 3A depicts a fragmentation of ω-5 Δ9-tetradecenoic acid after derivatization by dimethyl disulfide (DMDS).

FIG. 3B depicts the mass spectrum and ion fragmentation pattern of ω-5 Δ9-tetradecenoic acid after derivatization DMDS.

FIG. 4A depicts a fragmentation of ω-5 Δ11-hexadecenoic acid after derivatization by DMDS.

FIG. 4B depicts the mass spectrum and ion fragmentation pattern of ω-5 All-hexadecenoic acid after derivatization DMDS.

FIG. 5A depicts a fragmentation of ω-5 (z11)16-hydroxy-hexadecenoic acid after by derivatization DMDS.

FIG. 5B depicts the mass spectrum and ion fragmentation pattern of ω-5 z11-16-hydroxy-hexadecenoic acid after derivatization DMDS.

### DETAILED DESCRIPTION

### I. Definitions

The following definitions refer to the various terms used above and throughout the disclosure.

As used herein, singular articles such as “a” and “an” and “the” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Unless defined otherwise, technical and scientific terms used herein have the same meaning as commonly understood by a person of ordinary skill in the art. In particular, this disclosure utilizes routine techniques in the field of recombinant genetics, organic chemistry, fermentation and biochemistry.

The term “fatty acid” as used herein, refers to an aliphatic carboxylic acid having the formula RCOOH wherein R is an aliphatic group having at least 4 carbons, typically between about 4 and about 28 carbon atoms. The aliphatic R group can be saturated or unsaturated, branched or unbranched. Branched aliphatic R groups may include branches comprising lower alkyl branches, such as a C1-C4 alkyl, preferably in the ω-1 or ω-2 position. In some embodiments, the branched aliphatic R group may be methyl in the ω-1 or ω-2 position. Unsaturated fatty acids may be monounsaturated or polyunsaturated.

The fatty acid or fatty acids, as used herein, can be produced within a cell through the process of fatty acid biosynthesis, through the reverse of fatty acid degradation or beta-oxidation (β-oxidation), or they can be fed to a cell. As is well known in the art, fatty acid biosynthesis is generally a malonyl-CoA dependent synthesis of acyl-ACPs or acyl CoAs, while the reverse of beta-oxidation results is acetyl-CoA dependent and results in the synthesis of acyl-CoAs. Fatty acids fed to cell are converted to acyl-CoAs and can be converted to acyl-ACPs. Fatty acids can be synthesized in a cell by natural fatty acid biosynthetic pathways or can be synthesized from heterologous fatty acid biosynthetic pathways that comprise a combination of fatty acid biosynthetic and/or degradation enzymes that result in the synthesis of acyl-CoAs and/or Acyl-ACPs.

The term “fatty acid derivative” as used herein, refers to a product derived from a fatty acid. Thus, a fatty acid derivative is a compound that includes a fatty acid as defined above with a modification. In general, fatty acid derivatives include malonyl-CoA derived compounds including acyl-ACP or acyl-ACP derivatives. Thus, a fatty acid derivative includes alkyl-thioesters and acyl-thioesters. Further, a fatty acid derivative includes a molecule/compound that is derived from a metabolic pathway that includes a fatty acid derivative enzyme. Exemplary fatty acid derivatives include fatty acids, fatty acid esters (e.g., waxes), fatty acid methyl esters (FAME), fatty acid ethyl esters (FAEE)), fatty alcohol acetate esters (FACE), fatty amines, fatty aldehydes, fatty alcohols, hydrocarbons (e.g., alkanes, alkenes, etc.), ketones, terminal olefins, internal olefins, 3-hydroxy fatty acid derivatives, bifunctional fatty acid derivatives (e.g., ω-hydroxy fatty acids, (ω-1)-hydroxy fatty acids, (ω-2)-hydroxy fatty acids, (ω-3)-hydroxy fatty acids, 10-hydroxy fatty acids, 1,3 fatty-diols, α,ω-diols, α,ω-3-hydroxy triols, ω-hydroxy FAME, ω-OH FAEE, etc.), and unsaturated fatty acid derivatives, including unsaturated compounds of each of the above mentioned fatty acid derivatives.

The expression “fatty acid composition” as used herein, refers to a composition of mUFAs or derivatives thereof, for example a fatty acid composition produced by recombinant proteobacterium described herein, such as a recombinant proteobacterium comprising an acyl-ACP desaturase and an acyl-ACP thioesterase, wherein the native dual 3-hydroxy acyl-ACP dehydratase/isomerase is deleted. A fatty acid derivative composition may comprise a single fatty acid derivative species or may comprise a mixture of fatty acid derivative species. In some exemplary embodiments, the mixture of fatty acid derivatives includes more than one type of fatty acid derivative product (e.g., fatty acids, fatty acid esters, fatty alcohols, fatty alcohol acetates, fatty aldehydes, fatty amine, bifunctional fatty acid derivatives, and non-native monounsaturated fatty acid derivatives, etc.). In other exemplary embodiments, the mixture of fatty acid derivatives includes a mixture of non-native monounsaturated fatty acid esters (or another fatty acid derivatives) with different chain lengths, saturation and/or branching characteristics. In other exemplary embodiments, the mixture of fatty acid derivatives comprises predominantly one type of fatty acid derivative e.g., an ω-3-monounsaturated fatty acid or fatty acid derivative composition, an ω-5-monounsaturated fatty acid or fatty acid derivative composition, an ω-11-monounsaturated fatty acid or fatty acid derivative composition, etc. In still other exemplary embodiments, a fatty acid derivative composition comprises a mixture of more than one type of fatty acid derivative product e.g., fatty acid derivatives with different chain lengths, saturation and/or branching characteristics. In still other exemplary embodiments, a “fatty acid derivative composition” comprises a mixture of fatty esters and 3-hydroxy esters. In still other exemplary embodiments, a fatty acid derivative composition comprises a mixture of fatty alcohols and fatty aldehydes, for example a mixture of non-native monounsaturated fatty alcohols or fatty aldehydes. In other exemplary embodiments, the mixture of fatty acid derivatives includes a mixture of non-native monounsaturated fatty acid derivatives with different chain lengths, saturation and/or functional group characteristics.

The term “attenuated,” as used herein, refers to the expression of a protein or enzyme, such as dual 3-hydroxy acyl-ACP dehydratase/isomerase (FabA), which is reduced as compared to wild-type or conventional expression levels of the protein or enzyme in a wild-type proteobacteria. The degree of attenuation is not particularly limited and encompasses a reduction in expression of, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, and at least about 95%. Where expression of the protein or enzyme is completely abolished, the protein or enzyme is considered “deleted,” rather than “attenuated.”

The term “non-native monounsaturated fatty acid derivative” as used herein, refers to any mUFA derivative derived from a mUFA acyl-thioester where the double bond position is non-native to the producing cell (e.g., proteobacterium). For example, in E. coli the native double-bond position in monounsaturated fatty acid acyl-thioesters is omega-7 (ω-7). Therefore, for example in E. coli, a mUFA derivative derived from a mUFA acyl-thioester with a double bond in a position other than ω-7 is defined as a non-native mUFA derivative for this bacterium. Examples of non-native mUFA and derivatives thereof have double bonds at ω-3, ω-5, ω-6, ω-8, ω-9, ω-11, and/or ω-12.

Fatty acid compositions may comprise non-native mUFAs or derivatives thereof. Compositions comprising non-native mUFA or derivatives thereof produced by the recombinant proteobacteria disclosed herein typically comprise compositions wherein the non-native mUFA or derivative thereof is at least about 10% of the total mUFA (including native and non-native mUFAs) produced. In some embodiments, compositions comprising non-native mUFA or derivatives thereof produced by the recombinant proteobacteria disclosed herein comprise compositions wherein the non-native mUFA or derivative thereof is at least about 20% of the total mUFA or derivatives thereof. In other embodiments, compositions comprising non-native mUFA or derivatives thereof produced by the recombinant proteobacteria disclosed herein comprise compositions wherein the non-native mUFA or derivatives thereof is at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, at least about 99%, of the total mUFA or derivatives thereof.

Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers” or alternatively a simply “Accession Numbers”), and from the UniProt Knowledgebase (UniProtKB) and Swiss-Prot databases provided by the Swiss Institute of Bioinformatics (which are identified herein as “UniProtKB Accession Numbers”).

The term “enzyme classification (EC) number” refers to a number that denotes a specific polypeptide sequence or enzyme. EC numbers classify enzymes according to the reaction they catalyze. EC numbers are established by the nomenclature committee of the international union of biochemistry and molecular biology (IUBMB), a description of which is available on the IUBMB enzyme nomenclature website on the world wide web.

As used herein, the term “isolated,” with respect to products (such as mUFA derivatives disclosed herein) refers to products that are separated from cellular components, cell culture media, or chemical or synthetic precursors. The mUFA derivatives disclosed herein produced by the methods disclosed herein can be relatively immiscible in the fermentation broth, as well as in the cytoplasm. Therefore, in exemplary embodiments, the non-native mUFA derivatives disclosed herein collect in an organic phase extracellularly and are thereby “isolated”.

As used herein, the terms “polypeptide” and “protein” are used interchangeably to refer to a polymer of amino acid residues that is typically 12 or more amino acids in length. Polypeptides less than 12 amino acids in length are referred to herein as “peptides.” The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide. In some exemplary embodiments, DNA or RNA encoding an expressed peptide, polypeptide or protein is inserted into the host chromosome via homologous recombination or other means well known in the art, and is so used to transform a host cell to produce the peptide or polypeptide. Similarly, the terms “recombinant polynucleotide” or “recombinant nucleic acid” or “recombinant DNA” are produced by recombinant techniques that are known to those of skill in the art (see e.g., methods described in Sambrook et al. (Sambrook et al., Molecular Cloning—A Laboratory Manual, Cold Spring Harbor Press 4^{th} Edition (Cold Spring Harbor, N.Y. 2012) and/or Current Protocols in Molecular Biology (Volumes 1-3, John Wiley & Sons, Inc. (1994-1998) and Supplements 1-115 (1987-2016).).

When referring to two nucleotide or polypeptide sequences, the “percentage of sequence identity” between the two sequences is determined by comparing the two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The “percentage of sequence identity” is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Thus, the expression “percent identity,” or equivalently “percent sequence identity,” “homology, or “homologous” in the context of two or more nucleic acid sequences or peptides or polypeptides, refers to two or more sequences or subsequences that are the same or have a specified percentage of nucleotides or amino acids that are the same (e.g., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured e.g., using a BLAST or BLAST 2.0 sequence comparison algorithm with default parameters (see e.g., Altschul et al. (1990) J. Mol. Biol. 215(3):403-410) and/or the NCBI web site atncbi.nlm.nih.gov/BLAST/) or by manual alignment and visual inspection. Percent sequence identity between two nucleic acid or amino acid sequences also can be determined using e.g., the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent sequence identity between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial sequence identity calculations and adjust the algorithm parameters accordingly. A set of parameters that may be used if a practitioner is uncertain about which parameters should be applied to determine if a molecule is within a sequence identity limitation of the claims, are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272(20):5101-5109).

Two or more nucleic acid or amino acid sequences are said to be “substantially identical,” when they are aligned and analyzed as discussed above and are found to share about 50% identity, preferably 55%, 60%, 65%, 70%, 75%, 80%, 85%, 90%, 91%, 92%, 93%, 94%, 95%, 96%, 97%, 98%, 99%, or higher identity over a specified region. Two nucleic acid sequences or polypeptide sequences are said to be “identical” if the sequence of nucleotides or amino acid residues, respectively, in the two sequences are the same when aligned for maximum correspondence as described above. This definition also refers to, or may be applied to, the compliment of a test sequence. Identity is typically calculated over a region that is at least about 25 amino acids or nucleotides in length, or more preferably over a region that is 50-100 amino acids or nucleotides in length, or over the entire length of a given sequence.

The term “endogenous” as used herein refers to a substance e.g., a nucleic acid, protein, etc. that is produced from within a cell. Thus, an endogenous polynucleotide or polypeptide refers to a polynucleotide or polypeptide produced by the cell. In some exemplary embodiments an endogenous polypeptide or polynucleotide is encoded by the genome of the parental cell (or host cell). In other exemplary embodiments, an endogenous polypeptide or polynucleotide is encoded by an autonomously replicating plasmid carried by the parental cell (or host cell). In some exemplary embodiments, an endogenous gene is a gene that was present in the cell when the cell was originally isolated from nature i.e., the gene is native to the cell. In other exemplary embodiments, an “endogenous” gene has been altered through recombinant techniques e.g., by altering the relationship of control and/or coding sequences. Thus, a heterologous gene may, in some exemplary embodiments, be endogenous to a host cell. Additionally, a variant (i.e., mutant) polypeptide may be produced from with the and would be considered endogenous polypeptide.

In contrast, an “exogenous” polynucleotide or polypeptide, or other substance (e.g., fatty acid derivative, small molecule compound, etc.) refers to a polynucleotide or polypeptide or other substance that is not encoded or produced by the cell and which is therefore added to a cell, a cell culture, or assay from outside of the cell. A variant (i.e., mutant) polypeptide added to the cell, cell culture, or assay is one example of an exogenous polypeptide.

As used herein the term “native” refers to the form of a nucleic acid, protein, polypeptide, or a fragment thereof that is isolated from nature or a nucleic acid, protein, polypeptide, or a fragment thereof that is in its natural state without intentionally introduced mutations in the structural sequence and/or without any engineered changes in expression such as e.g., changing a developmentally regulated gene to a constitutively expressed gene. As used herein, “native” also refers to “wildtype” or “wild-type,” in which the nucleic acid, protein, polypeptide, or a fragment thereof is present in both sequence, quantity, and relative quantity as typically found in the organism as naturally found. Wild-type organisms may serve as a control and/or reference for determination of cellular functions, such as identity and/or quantity of mUFA(s) produced or relative binding of heterologous regulatory elements (e.g., FadR) to cognate DNA sequences.

The term “non-native” is used herein to refer to nucleic acid sequences, amino acid sequences, fatty acids, and derivatives thereof, and/or small molecules that do not occur naturally in the host. Heterologous genes are considered “non-native.” A nucleic acid sequence or amino acid sequence that has been removed from a host cell, subjected to laboratory manipulation, and introduced or reintroduced into a host cell is considered “non-native.” Synthetic or partially synthetic genes introduced into a host cell are “non-native.” Non-native genes further include genes endogenous and/or native to the host microorganism but operably linked to one or more heterologous regulatory sequences that have been recombined into the host genome. A naturally occurring gene under the control of a heterologous regulatory sequence is considered “non-native.”

The term “gene” as used herein, refers to nucleic acid sequences e.g., DNA sequences, which encode either an RNA product or a protein product, as well as operably-linked nucleic acid sequences that affect expression of the RNA or protein product (e.g., expression control sequences such as e.g., promoters, enhancers, ribosome binding sites, translational control sequences, etc.). The term “gene product” refers to either the RNA (e.g., tRNA, mRNA) and/or protein expressed from a particular gene.

The term “expression” or “expressed” as used herein in reference to a gene, refers to the production of one or more transcriptional and/or translational product(s) of a gene. In exemplary embodiments, the level of expression of a DNA molecule in a cell is determined on the basis of either the amount of corresponding mRNA that is present within the cell or the amount of protein encoded by that DNA produced by the cell. The term “expressed genes” refers to genes that are transcribed into messenger RNA (mRNA) and then translated into protein, as well as genes that are transcribed into other types of RNA, such as e.g., transfer RNA (tRNA), ribosomal RNA (rRNA), and regulatory RNA, which are not translated into protein.

The level of expression of a nucleic acid molecule in a cell or cell free system is influenced by “expression control sequences” or equivalently “regulatory sequences” or “regulatory elements.” Expression control sequences, regulatory sequences, or regulatory elements are known in the art and include, for example, promoters, enhancers, polyadenylation signals, transcription terminators, nucleotide sequences that affect RNA stability, internal ribosome entry sites (IRES), and the like, that provide for the expression of the polynucleotide sequence in a host cell. In exemplary embodiments, “expression control sequences” interact specifically with cellular proteins involved in transcription (see e.g., Maniatis et al., Science, 236: 1237-1245 (1987); Goeddel, Gene Expression Technology: Methods in Enzymology, Vol. 185, Academic Press, San Diego, Calif. (1990)). In exemplary methods, an expression control sequence, regulatory sequence, or regulatory element is operably linked to a polynucleotide sequence. By “operably linked” is meant that a polynucleotide sequence and an expression control sequence(s) or regulatory element(s) are functionally connected so as to permit expression of the polynucleotide sequence when the appropriate molecules (e.g., transcriptional activator proteins) contact the expression control sequence(s). In exemplary embodiments, operably linked promoters are located upstream of the selected polynucleotide sequence in terms of the direction of transcription and translation. In some exemplary embodiments, operably linked enhancers can be located upstream, within, or downstream of the selected polynucleotide.

As used herein, the phrase “expression of said nucleotide sequence is modified relative to the wild-type nucleotide sequence,” refers to a change e.g., an increase or decrease in the level of expression of a native nucleotide sequence or a change e.g., an increase or decrease in the level of the expression of a heterologous or non-native polypeptide-encoding nucleotide sequence as compared to a control nucleotide sequence e.g., wild-type control. In some exemplary embodiments, the phrase “the expression of said nucleotide sequence is modified relative to the wild type nucleotide sequence,” refers to a change in the pattern of expression of a nucleotide sequence as compared to a control pattern of expression e.g., constitutive expression as compared to developmentally timed expression.

A “control” sample (e.g., a control nucleotide sequence, a control polypeptide sequence, a control cell, etc., or value) refers to a sample that serves as a reference, usually a known reference, for comparison to a test sample. For example, in an exemplary embodiment, a test sample comprises a non-native monounsaturated fatty acid derivative composition made by a recombinant proteobacterium that comprises a heterologous acyl-ACP desaturase and a heterologous acyl-ACP thioesterase, wherein the native dual 3-hydroxy acyl-ACP dehydratase/isomerase (Fad A) is deleted, as disclosed herein, while the control sample comprises a non-native monounsaturated fatty acid derivative composition made by the corresponding or designated bacterium that does not comprise a heterologous acyl-ACP desaturase and a heterologous acyl-ACP thioesterase and does not have its native dual 3-hydroxy acyl-ACP dehydratase/isomerase (Fad A) deleted. Additionally, a control cell or microorganism may be referred to as a corresponding wild type or host cell. One of skill will recognize that controls can be designed for assessment of any number of parameters. Furthermore, one of skill in the art will understand which controls are valuable in a given situation and will be able to analyze data based on comparisons to control values.

The terms “overexpressed” or “up-regulated” as used herein, refer to a gene whose expression is elevated in comparison to a control level of expression. In exemplary embodiments, overexpression of a gene is caused by an elevated rate of transcription as compared to the native transcription rate for that gene. In other exemplary embodiments, overexpression is caused by an elevated rate of translation of the gene compared to the native translation rate for that gene. Methods of testing for overexpression are well known in the art, for example transcribed RNA levels can be assessed using rtPCR and protein levels can be assessed using SDS page gel analysis.

In other embodiments, the polypeptide, polynucleotide, or hydrocarbon having an altered level of expression is “attenuated” or has a “decreased level of expression” or is “down-regulated.” As used herein, these terms mean to express or cause to be expressed a polynucleotide, polypeptide, or hydrocarbon in a cell at a lesser concentration than is normally expressed in a corresponding control cell (e.g., wild type cell) under the same conditions. In other words, the term “attenuate” means to weaken, reduce, or diminish. For example, a polypeptide can be attenuated by modifying the polypeptide to reduce its activity (e.g., by modifying a nucleotide sequence that encodes the polypeptide).

A polynucleotide or polypeptide can be attenuated using any method known in the art. For example, in some exemplary embodiments, the expression of a gene or polypeptide encoded by the gene is attenuated by mutating the regulatory polynucleotide sequences which control expression of the gene. In other exemplary embodiments, the expression of a gene or polypeptide encoded by the gene is attenuated by overexpressing a repressor protein, or by providing an exogenous regulatory element that activates a repressor protein. In still other exemplary embodiments, DNA- or RNA-based gene silencing methods are used to attenuate the expression of a gene or polynucleotide. In some embodiments, the expression of a gene or polypeptide is completely attenuated, e.g., by deleting all or a portion of the polynucleotide sequence of a gene.

The degree of overexpression or attenuation can be 1.5-fold or more, e.g., 2-fold or more, 3-fold or more, 5-fold or more, 10-fold or more, or 15-fold or more. Alternatively, or in addition, the degree of overexpression or attenuation can be 500-fold or less, e.g., 100-fold or less, 50-fold or less, 25-fold or less, or 20-fold or less. Thus, the degree of overexpression or attenuation can be bounded by any two of the above endpoints. For example, the degree of overexpression or attenuation can be 1.5-500-fold, 2-50-fold, 10-25-fold, or 15-20-fold.

As used herein, “modified activity” or an “altered level of activity” of a protein/polypeptide in a recombinant host cell refers to a difference in one or more characteristics in the activity the protein/polypeptide as compared to the characteristics of an appropriate control protein e.g., the corresponding parent protein or corresponding wild type protein. Thus, in exemplary embodiments, a difference in activity of a protein having “modified activity” as compared to a corresponding control protein is determined by measuring the activity of the modified protein in a recombinant host cell and comparing that to a measure of the same activity of a corresponding control protein in an otherwise isogenic host cell. Modified activities can be the result of, for example, changes in the structure of the protein (e.g., changes to the primary structure, such as e.g., changes to the protein's nucleotide coding sequence that result in changes in substrate specificity, changes in observed kinetic parameters, changes in solubility, etc.); changes in protein stability (e.g., increased or decreased degradation of the protein) etc.

The term “heterologous” as used herein refers to a polypeptide or polynucleotide which is in a non-native state. Thus, a polynucleotide or a polypeptide is “heterologous” to a cell when the polynucleotide and/or the polypeptide and the cell are not found in the same relationship to each other in nature. Therefore, a polynucleotide or polypeptide sequence is “heterologous” to an organism or a second sequence if it originates from a different organism, different cell type, or different species, or, if from the same species, it is modified from its original form. Thus, in an exemplary embodiment, a polynucleotide or polypeptide is “heterologous” when it is not naturally present in a given organism. For example, a polynucleotide sequence that is native to cyanobacteria can be introduced into a host cell of E. coli by recombinant methods, and the polynucleotide from cyanobacteria is then heterologous to the E. coli cell (i.e., the now recombinant E. coli cell).

Similarly, a polynucleotide or polypeptide is heterologous when it is modified from its native form or from its relationship with other polynucleotide sequences or is present in a recombinant host cell in a non-native state. Thus, in an exemplary embodiment, a heterologous polynucleotide or polypeptide comprises two or more subsequences that are not found in the same relationship to each other in nature. For example, a promoter operably linked to a nucleotide coding sequence derived from a species different from that from which the promoter was derived. Alternatively, in another example, if a promoter is operably linked to a nucleotide coding sequence derived from a species that is the same as that from which the promoter was derived, then the operably-linked promoter and coding sequence are “heterologous” if the coding sequence is not naturally associated with the promoter (e.g., a constitutive promoter operably linked to a developmentally regulated coding sequence that is derived from the same species as the promoter). In other exemplary embodiments, a heterologous polynucleotide or polypeptide is modified relative to the wild type sequence naturally present in the corresponding wild type host cell, e.g., an intentional modification e.g., an intentional mutation in the sequence of a polynucleotide or polypeptide or a modification in the level of expression of the polynucleotide or polypeptide. Typically, a heterologous nucleic acid or polynucleotide is recombinantly produced.

The term “recombinant” as used herein, refers to a genetically modified polynucleotide, polypeptide, cell, tissue, or organism. When used with reference to a cell, the term “recombinant” indicates that the cell has been modified by the introduction of a heterologous nucleic acid or protein or has been modified by alteration of a native nucleic acid or protein, or that the cell is derived from a cell so modified and that the derived cell comprises the modification. Thus, for example, “recombinant cells” or equivalently “recombinant host cells” may be modified to express genes that are not found within the native (non-recombinant) form of the cell or may be modified to abnormally express native genes e.g., native genes may be overexpressed, underexpressed or not expressed at all. In exemplary embodiments, a “recombinant cell” or “recombinant host cell” is engineered to express a heterologous enzyme pathway capable of producing a bifunctional fatty acid derivative molecule. A recombinant cell can be derived from a microorganism such as a bacterium, proteobacterium, archaea, a virus or a fungus. In addition, a recombinant cell can be derived from a plant or an animal cell. In exemplary embodiments, a “recombinant host cell” or “recombinant cell” is used to produce one or more non-native monounsaturated fatty acid derivatives including, but not limited to, non-native monounsaturated fatty acids, non-native monounsaturated fatty esters (e.g., waxes), fatty acid esters, fatty esters, fatty acid methyl esters (FAME), fatty acid ethyl esters (FAEE)), non-native monounsaturated fatty acyl acetate esters (FACE), non-native monounsaturated fatty alcohols (e.g., polyols), non-native monounsaturated fatty aldehydes, non-native monounsaturated fatty amines, non-native monounsaturated terminal olefins, non-native monounsaturated ketones, etc. Therefore, in some exemplary embodiments a “recombinant host cell” is a “production host” or equivalently, a “production host cell”. In some exemplary embodiments, the recombinant cell includes one or more polynucleotides, each polynucleotide encoding a polypeptide having fatty acid biosynthetic enzyme activity, wherein the recombinant cell produces a non-native monounsaturated fatty acid derivative composition when cultured in the presence of a (simple) carbon source under conditions effective to express the polynucleotides.

When used with reference to a polynucleotide, the term “recombinant” indicates that the polynucleotide has been modified by comparison to the native or naturally occurring form of the polynucleotide or has been modified by comparison to a naturally occurring variant of the polynucleotide. In an exemplary embodiment, a recombinant polynucleotide (or a copy or complement of a recombinant polynucleotide) is one that has been manipulated by the hand of man to be different from its naturally occurring form. Thus, in an exemplary embodiment, a recombinant polynucleotide is a mutant form of a native gene or a mutant form of a naturally occurring variant of a native gene wherein the mutation is made by intentional human manipulation e.g., made by saturation mutagenesis using mutagenic oligonucleotides, through the use of UV radiation, mutagenic chemicals, chemical synthesis, etc. Such a recombinant polynucleotide might comprise one or more point mutations, deletions and/or insertions relative to the native or naturally occurring variant form of the gene. Similarly, a polynucleotide comprising a promoter operably linked to a second polynucleotide (e.g., a coding sequence) is a “recombinant” polynucleotide. Thus, a recombinant polynucleotide comprises polynucleotide combinations that are not found in nature. A recombinant protein (discussed supra) is typically one that is expressed from a recombinant polynucleotide, and recombinant cells, tissues, and organisms are those that comprise recombinant sequences (polynucleotide and/or polypeptide).

The term “vector,” as used herein, refers to a polynucleotide sequence that contains a gene of interest (e.g., encodes one or more proteins or enzymes described herein) and a promoter operably linked to the fatty acid biosynthetic polynucleotide sequence of interest. Once a polynucleotide sequence(s) encoding a fatty acid biosynthetic pathway polypeptide has been prepared and isolated, various methods may be used to construct expression cassettes, vectors, and other DNA constructs. The skilled artisan is well aware of the genetic elements that must be present on an expression construct/vector in order to successfully transform, select and propagate the expression construct in host cells. Techniques for manipulation of nucleic acids such as subcloning nucleic acid sequences into expression vectors, labeling probes, DNA hybridization are well known in the art.

As used herein, the term “microorganism” refers generally to a microscopic organism. Microorganisms can be prokaryotic or eukaryotic. Exemplary prokaryotic microorganisms include e.g., bacteria (including γ-proteobacteria), archaea, cyanobacteria, etc. An exemplary proteobacterium is Escherichia coli. Exemplary eukaryotic microorganisms include e.g., yeast, protozoa, algae, etc. In exemplary embodiments, a “recombinant microorganism” is a microorganism that has been genetically altered and thereby expresses or encompasses a heterologous nucleic acid sequence and/or a heterologous protein.

The expression “viable bacterium” or “viable bacteria” or “viable microorganism” as used herein, refers to a bacterium (including proteobacterium) that grows on a carbon source e.g., a simple carbon source, wherein the media used for culturing the microorganism does not contain any exogenous fatty acid or fatty acid derivative, nor any compound(s) that is/are an inhibitor of fatty acid biosynthesis, e.g., triclosan, which inhibits the fabI-type trans-2-enoyl-ACP reductase. Typically, as used herein, a viable recombinant bacterium, including a recombinant proteobacterium, comprises at least one heterologous acyl-ACP desaturase and at least one heterologous acyl-ACP thioesterase, and wherein 3-hydroxy acyl-ACP the native dual 3-hydroxy acyl-ACP dehydratase/isomerase (FabA) is deleted or attenuated. In some embodiments, the recombinant proteobacterium is a gamma proteobacterium (also known as a γ-proteobacterium). In some embodiments, the recombinant proteobacterium may be Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, Marinobacter aquaeolei, Yersinia pestis, or Vibrio cholerae.

As used herein, the term “culture” typical refers to a liquid media comprising viable cells. In one embodiment, a culture comprises cells reproducing in a predetermined culture media under controlled conditions, for example, a culture of recombinant host cells grown in liquid media comprising a selected carbon source and nitrogen.

“Culturing” or “cultivation” refers to growing a population of recombinant host cells under suitable conditions in a liquid or solid medium. In particular embodiments, culturing refers to the fermentative bioconversion of a substrate to an end-product. Culturing media are well known, and individual components of such culture media are available from commercial sources, e.g., under the Difco™ and BBL™ trademarks. In one non-limiting example, the aqueous nutrient medium is a “rich medium” comprising complex sources of nitrogen, salts, and carbon, such as YP medium, comprising 10 g/L of peptone and 10 g/L yeast extract of such a medium.

Typically, a “recombinant proteobacterium” as disclosed herein will comprise within its cellular fatty acids/membrane phospholipids the non-native monounsaturated fatty acid (or fatty acid derivative) produced by the cell that has the characteristic double bond structure. In some embodiments, the non-native monounsaturated fatty acid derivative comprises at least 5% of the membrane phospholipids. In other embodiments, the non-native monounsaturated fatty acid derivative comprises at least 10% of the membrane phospholipids. In still other embodiments, the non-native monounsaturated fatty acid derivative comprises at least 11%, at least 12%, at least 15%, at least 20%, at least 25%, at least 30%, at least 35%, at least 40%, of the membrane phospholipids. In another embodiment, the recombinant proteobacterium will comprise a free fatty acid composition of at least 11%, at least 12%, at least 15%, at least 20%, at least 25%, at least 30%, at least 35%, at least 40%, at least 50%, at least 60%, or at least 70%, non-native monounsaturated fatty acid.

A “production host” or equivalently a “production host cell” is a cell used to produce products. As disclosed herein, a production host is typically modified to express or overexpress selected genes, or to have attenuated expression of selected genes. Thus, a production host or a production host cell is a recombinant host or equivalently a recombinant host cell. Non-limiting examples of production hosts include e.g., recombinant proteobacteria as disclosed above. An exemplary production host is a recombinant proteobacterium comprising a heterologous acyl-ACP desaturase and a heterologous acyl-ACP thioesterase, wherein the native dual 3-hydroxy acyl-ACP dehydratase/isomerase (FabA) is deleted or attenuated.

As used herein, the terms “purify,” “purified,” or “purification” mean the removal or isolation of a molecule from its environment by, for example, isolation or separation. “Substantially purified” molecules are at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, at least about 99% free) from other components with which they are associated. As used herein, these terms also refer to the removal of contaminants from a sample.

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; succinate, lactate, and acetate; alcohols, such as ethanol, methanol, and glycerol, or mixtures thereof. The carbon source can also be a product of photosynthesis, such as glucose. In certain embodiments, the carbon source is biomass. In other embodiments, the carbon source is glucose. In other embodiments the carbon source is sucrose. In other embodiments the carbon source is glycerol. In other embodiments, the carbon source is a simple carbon source such as e.g., glucose. In other embodiments, the carbon source is a renewable carbon source. In other embodiment, the carbon source is natural gas. In other embodiments the carbon source comprises one or more components of natural gas, such as methane, ethane, or propane. In other embodiments, the carbon source is flu gas or synthesis gas. In still other embodiments, the carbon source comprises one or more components of flu or synthesis gas such as carbon monoxide, carbon dioxide, hydrogen, etc. As used herein, the term “carbon source” or “simple carbon source” specifically excludes oleochemicals such as e.g., saturated or unsaturated fatty acids.

### Enzymes

As used herein, the term “acyl-ACP desaturase” refers to an enzyme that belongs in the oxidoreductase family of enzymes and catalyzes the reaction of saturated acyl-ACP to cis-monounsaturated acyl-ACP. For example, the saturated acyl-ACP may be stearoyl-ACP and the cis-monounsaturated acyl-ACP may be oleoyl-ACP. The acyl-ACP desaturase may be native to the cell (e.g., present in the cell in its natural state without any intentionally induced mutations or changes to its expression) or may be heterologous (e.g., present in cell in an unnatural state, for example the enzyme is intentionally mutated or has an intentionally altered expression). In some embodiments, wherein the acyl-ACP desaturase is heterologous, it may be endogenous, wherein the enzyme, or a polynucleotide encoding the enzyme (e.g., RNA) is produced by the cell. In another embodiment, wherein the acyl-ACP desaturase is heterologous, it may be exogenous, wherein the enzyme or a polynucleotide encoding the enzyme is not produced by the cell, but instead is added to the cell from outside the cell. In some embodiments, the acyl-ACP desaturases described herein may belong to EC 1.14.19.2. The particular acyl-ACP desaturase determines where the cis double bond in the mUFA or derivative is located. In a particular embodiment, the acyl-ACP desaturase is Δ9-tetradecanoyl-acyl-ACP desaturase. In a further embodiment, the acyl-ACP desaturase has about an 85% sequence identity to SEQ ID NO: 2. In still further embodiments, the acyl-ACP desaturase has about a 90% sequence identity to SEQ ID NO: 2, about a 95% sequence identity to SEQ ID NO: 2, about a 99% sequence identity to SEQ ID NO: 2, or is SEQ ID NO: 2 (e.g., 100% sequence homology). In another embodiment, the acyl-ACP desaturase has about an 85% sequence identity to SEQ ID NO: 12. In still further embodiments, heterologous acyl-ACP desaturase has about a 90% sequence identity to SEQ ID NO: 12, about a 95% sequence identity to SEQ ID NO: 12, about a 99% sequence identity to SEQ ID NO: 12, or is SEQ ID NO: 12 (e.g., 100% sequence homology).

As used herein, the term “acyl-ACP thioesterase” refers to an enzyme that catalyzes the hydrolysis of thioester bonds to terminate fatty acyl extension. The acyl-ACP thioesterase may be native to the recombinant proteobacterium or may be heterologous. In some embodiments, the heterologous acyl-ACP thioesterase may be endogenous, wherein the enzyme, or a polynucleotide encoding the enzyme (e.g., RNA) is produced by the cell. In another embodiment, the heterologous acyl-ACP thioesterase may be exogenous, wherein the enzyme or a polynucleotide encoding the enzyme is not produced by the cell, but instead is added to the cell from outside the cell. In some embodiments, the acyl-ACP thioesterases described herein may belong to EC 3.1.2.14. The particular acyl-ACP thioesterase determines the chain length of the mUFA or derivative thereof. In a particular embodiment, the acyl-ACP thioesterase is a plant FatA-type thioesterase (which is specific for acyl-ACPs with C16 and C18 chain lengths), a plant FatB-type thioesterase (which is specific for acyl-ACPs with a C14 chain length), or a bacterial acyl-ACP thioesterase (for example, the thioesterase from C. thermocellum that hydrolyzes acyl-ACPs of various chain lengths, including z9-tetradecanol). In a further embodiment, the acyl-ACP thioesterase has about an 85% sequence identity to SEQ ID NO: 8. In still further embodiments, the acyl-ACP thioesterase has about a 90% sequence identity to SEQ ID NO: 8, about a 95% sequence identity to SEQ ID NO: 8, about a 99% sequence identity to SEQ ID NO: 8, or is SEQ ID NO: 8 (e.g., 100% sequence homology).

E. coli has two 3-hydroxy-acyl-ACP dehydratases, FabZ and FabA [EC 4.2.1.59]. A key enzyme for mUFA synthesis is one of these enzymes, FabA, which is a dual function dehydratase/isomerase. FabA isomerizes trans-2-decenoyl-ACP to cis-3-decenoyl-ACP. The latter cannot be reduced by trans-2-enoyl-ACP reductase, FabI, but can be elongated by R-keto-acyl-ACP synthase I, FabB, thereby fixating the double bond position in the ω-7 position (i.e., native position, counted from the reduced end of the acyl chain) of mUFAs with chain length C10, C12, C14, C16 and C18. It should be noted that the common chemical nomenclature designates the double bond position in fatty acids counting from the carboxyl group and not from the reducing end, and it uses (z) for cis- and (e) for trans-configuration, accordingly the native mUFAs in E. coli are z3-C10:1 (=ω7-C10:1), z5-C12:1 (=ω7-C12:1), C14:1 (=ω7-C14:1), z9-C16:1 (=ω7-C16:1) and z11-C18:1 (=ω7-C18:1). Thus, the expression “dual 3-hydroxy acyl-ACP dehydratase/isomerase” as used herein, refers to an enzyme having (3-hydroxyacyl-[acyl-carrier-protein] dehydratase) activity described by EC number: EC 4.2.1.59 and enoyl-acyl-carrier protein isomerase activity described by EC number: EC 5.3.3.14 (see e.g., Heath RJ, Rock CO (1996) Roles of the FabA and FabZ beta-hydroxyacyl-acyl carrier protein dehydratases in Escherichia coli fatty acid biosynthesis. J Biol Chem 271:27795-801). The dual 3-hydroxy acyl-ACP dehydratase/isomerase may be native to the recombinant proteobacterium or may be heterologous. In some embodiments, the heterologous 3-hydroxy acyl-ACP dehydratase may be endogenous, wherein the enzyme, or a polynucleotide encoding the enzyme (e.g., RNA), is produced by the cell. In another embodiment, the dual 3-hydroxy acyl-ACP dehydratase/isomerase may be exogenous, wherein the enzyme or a polynucleotide encoding the enzyme is not produced by the cell, but instead is added to the cell from outside the cell. In some embodiments, the 3-hydroxy acyl-ACP dehydratase described herein may belong to EC 4.2.1.59.

As used herein, the term “carboxylic acid reductase” refers to an enzyme that converts a fatty acid to its corresponding aldehyde. The carboxylic acid reductase may be native to the recombinant proteobacterium or may be heterologous. In some embodiments, the heterologous carboxylic acid reductase may be endogenous, wherein the enzyme, or a polynucleotide encoding the enzyme (e.g., RNA), is produced by the cell. In another embodiment, the heterologous carboxylic acid reductase may be exogenous, wherein the enzyme or a polynucleotide encoding the enzyme is not produced by the cell, but instead is added to the cell from outside the cell. In some embodiments, the carboxylic acid reductase described herein may belong to EC 1.2.1.30. In a further embodiment, the carboxylic acid reductase has about an 85% sequence identity to SEQ ID NO: 18. In still further embodiments, the carboxylic acid reductase has about a 90% sequence identity to SEQ ID NO: 18, about a 95% sequence identity to SEQ ID NO: 18, about a 99% sequence identity to SEQ ID NO: 18, or is SEQ ID NO: 18 (e.g., 100% sequence homology).

As used herein, the term “ferredoxin” refers to an iron-sulfur protein that mediates the transfer of electrons in metabolic reactions. The ferredoxin may be native to the recombinant proteobacterium or may be heterologous. In some embodiments, the heterologous ferredoxin may be endogenous, wherein the enzyme, or a polynucleotide encoding the enzyme (e.g., RNA), is produced by the cell. In another embodiment, the heterologous ferredoxin may be exogenous, wherein the enzyme or a polynucleotide encoding the enzyme is not produced by the cell, but instead is added to the cell from outside the cell. In some embodiments ferredoxin refers to PetF, ferredoxin reductase (PetH), and flavodoxin/ferredoxin-NADP⁺ reductase (Fpr). In some embodiments, the ferredoxin described herein may belong to EC 1.18.1.2.

As used herein, the term “alcohol dehydrogenase” refers to an enzyme that catalyzes the interconversion between aliphatic alcohols (e.g., aliphatic medium-chain alcohols) and their corresponding aldehydes. The alcohol dehydrogenase may be native to the recombinant proteobacterium or may be heterologous. In some embodiments, and under some conditions, the alcohol dehydrogenase converts an alcohol into an aldehyde. In some embodiments and under some conditions, the alcohol dehydrogenase converts an aldehyde into an alcohol. In some embodiments, the heterologous alcohol dehydrogenase may be endogenous, wherein the enzyme, or a polynucleotide encoding the enzyme (e.g., RNA), is produced by the cell. In another embodiment, the heterologous alcohol dehydrogenase may be exogenous, wherein the enzyme or a polynucleotide encoding the enzyme is not produced by the cell, but instead is added to the cell from outside the cell. In some embodiments, the alcohol dehydrogenase may belong to EC 1.1.1.1. In a further embodiment, the alcohol dehydrogenase has about an 85% sequence identity to SEQ ID NO: 20. In still further embodiments, the alcohol dehydrogenase has about a 90% sequence identity to SEQ ID NO: 20, about a 95% sequence identity to SEQ ID NO: 20, about a 99% sequence identity to SEQ ID NO: 20, or is SEQ ID NO: 20 (e.g., 100% sequence homology).

As used herein, the term “ω-hydroxylase” refers to an enzyme that hydrolyzes a fatty acid derivative in the ω-position. The ω-hydroxylase may be native to the recombinant proteobacterium or may be heterologous. In some embodiments, the heterologous ω-hydroxylase may be endogenous, wherein the enzyme, or a polynucleotide encoding the enzyme (e.g., RNA) is produced by the cell. In another embodiment, the heterologous ω-hydroxylase may be exogenous, wherein the enzyme or a polynucleotide encoding the enzyme is not produced by the cell, but instead is added to the cell from outside the cell. In some embodiments, the ω-hydroxylase may belong to EC 1.14.15.3 or 1.14.14.80. In a further embodiment, the ω-hydroxylase may be a hybrid-fusion P450 enzyme or variant thereof such as one disclosed in WO 2014/201474 or WO 2017/106205, which are incorporated herein by reference in their entirety. In a particular embodiment the ω-hydroxylase has about an 85% sequence identity to SEQ ID NO: 22. In still further embodiments, the ω-hydroxylase has about a 90% sequence identity to SEQ ID NO: 22, about a 95% sequence identity to SEQ ID NO: 22, about a 99% sequence identity to SEQ ID NO: 22, or is SEQ ID NO: 22 (e.g., 100% sequence homology).

As used herein, the term “alcohol acetyl-CoA transferase” refers to an enzyme that converts an alcohol to an acetyl ester. The alcohol acetyl-CoA transferase may be native to the recombinant proteobacterium or may be heterologous. In some embodiments, the heterologous alcohol acetyl-CoA transferase may be endogenous, wherein the enzyme, or a polynucleotide encoding the enzyme (e.g., RNA), is produced by the cell. In another embodiment, the heterologous alcohol acetyl-CoA transferase may be exogenous, wherein the enzyme or a polynucleotide encoding the enzyme is not produced by the cell, but instead is added to the cell from outside the cell. In some embodiments, the alcohol acetyl-CoA transferase may belong to EC 2.3.1.84. In a further embodiment, the alcohol acetyl-CoA transferase has about an 85% sequence identity to SEQ ID NO: 24. In still further embodiments, the alcohol acetyl-CoA transferase has about a 90% sequence identity to SEQ ID NO: 24, about a 95% sequence identity to SEQ ID NO: 24, about a 99% sequence identity to SEQ ID NO: 24, or is SEQ ID NO: 24 (e.g., 100% sequence homology).

As used herein, the term “phosphopantetheinyl transferase” refers to an enzyme that transfers the 4′-phosphopantetheine moiety from CoA to the acyl carrier protein. The phosphopantetheinyl transferase may be native to the recombinant proteobacterium or may be heterologous. For example, the entD gene codes for a phosphopantetheinyl transferase. Overexpression of native E. coli entD, a phosphopantetheinyl transferase, enables the activation of carboxylic acid reductase CarB from apo-CarB to holo-CarB, thereby allowing conversion of free fatty acids into fatty aldehydes, which can then be converted to fatty alcohols by a fatty aldehyde reductase. See, for example, U.S. Pat. No. 9,340,801, which is incorporated herein by reference. In some embodiments, the heterologous phosphopantetheinyl transferase may be endogenous, wherein the enzyme, or a polynucleotide encoding the enzyme (e.g., RNA), is produced by the cell. In another embodiment, the heterologous phosphopantetheinyl transferase may be exogenous, wherein the enzyme or a polynucleotide encoding the enzyme is not produced by the cell, but instead is added to the cell from outside the cell. In some embodiments, the phosphopantetheinyl transferase may belong to EC 2.7.8.7. In a further embodiment, the phosphopantetheinyl transferase has about an 85% sequence identity to SEQ ID NO: 26. In still further embodiments, the phosphopantetheinyl transferase has about a 90% sequence identity to SEQ ID NO: 26, about a 95% sequence identity to SEQ ID NO: 26, about a 99% sequence identity to SEQ ID NO: 26, or is SEQ ID NO: 26 (e.g., 100% sequence homology).

As used herein, the term “aldehyde dehydrogenase” refers to enzymes that converts aldehydes to carboxylic acids. The aldehyde dehydrogenasee may be native to the recombinant proteobacterium or may be heterologous. In some embodiments, the heterologous aldehyde dehydrogenase may be endogenous, wherein the enzyme, or a polynucleotide encoding the enzyme (e.g., RNA) is produced by the cell. In another embodiment, the heterologous aldehyde dehydrogenase may be exogenous, wherein the enzyme or a polynucleotide encoding the enzyme is not produced by the cell, but instead is added to the cell from outside the cell. Aldehyde dehydrogenase may be described by the number EC 1.2.1.3. In a further embodiment, the heterologous aldehyde dehydrogenase has about an 85% sequence identity to one of SEQ ID NOs:30-32. In still further embodiments, the aldehyde dehydrogenase has about a 90% sequence identity to one of SEQ ID NOs: 30-32, about a 95% sequence identity to one of SEQ ID NOs: 30-32, about a 99% sequence identity to one of SEQ ID NOs: 30-32, or is one of SEQ ID NOs: 30-32 (e.g., 100% sequence homology).

As used herein, the term “O-ketoacyl-ACP-synthase,” which includes “FabB” or “FabF,” refers to enzymes that catalyzes the condensation reaction to elongate the fatty acid chain. The β-ketoacyl-ACP synthase may be native to the recombinant proteobacterium or may be heterologous. In some embodiments, the heterologous β-ketoacyl-ACP-synthase may be endogenous, wherein the enzyme or a polynucleotide encoding the enzyme (e.g., RNA) is produced by the cell. In another embodiment, the heterologous β-ketoacyl-ACP-synthase may be exogenous, wherein the enzyme or a polynucleotide encoding the enzyme is not produced by the cell, but instead is added to the cell from outside the cell. β-ketoacyl-ACP-synthase may be described by the number EC 2.3.1.41. In a further embodiment, the β-ketoacyl-ACP-synthase has about an 85% sequence identity to SEQ ID NO:33. In still further embodiments, the β-ketoacyl-ACP-synthase has about a 90% sequence identity to SEQ ID NO: 33, about a 95% sequence identity to SEQ ID NO: 33, about a 99% sequence identity to SEQ ID NO: 33, or is SEQ ID NO: 33 (e.g., 100% sequence homology).

### II. Recombinant Proteobacteria and Novel Δ9-Tetradecanoyl-Acyl-ACP Desaturase

As discussed above, there is a need for new and efficient recombinant methods for producing non-native mUFAs and derivatives thereof. Thus, in one embodiment a recombinant proteobacterium is described herein which produces non-native mUFAs and derivatives thereof comprising a heterologous acyl-ACP desaturase and a heterologous acyl-ACP thioesterase, wherein the native dual 3-hydroxy acyl-ACP dehydratase/isomerase (FabA) is deleted or attenuated. In some embodiments, the recombinant proteobacterium is a gamma-proteobacterium (e.g., γ-proteobacterium). In a further embodiment, the recombinant proteobacterium may be Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, Marinobacter aquaeolei, Yersinia pestis, or Vibrio cholerae. In a particular embodiment, the recombinant proteobacterium is E. coli.

The described recombinant proteobacterium can produce one or more non-native mUFAs or derivatives thereof, such as a ω-3, ω-5, ω-6, ω-8, ω-9, ω-11, ω-12, etc. mUFAs or derivatives thereof. In a particular embodiment, the recombinant proteobacterium produces an ω-5 mUFA or derivative thereof.

In another particular embodiment, the heterologous acyl-ACP desaturase is the newly discovered Δ9-tetradecanoyl-acyl-ACP desaturase from Vitis Vinifera having at least 85% sequence identity to SEQ ID NO:12, or the Δ9-tetradecanoyl-acyl-ACP desaturase from Pelargonium xhortorum having at least 85% sequence identity to SEQ ID NO:2. Some desaturases, mainly found in plants, act on acyl-ACPs. Desaturases that act on acyl-ACP substrates are both soluble and enable altering the cis double bond position in the acyl-ACP other than at the naturally occurring ω-7 position. Without being bound by theory, when an acyl-ACP desaturase is expressed in proteobacteria, the organism can produce mUFAs and derivatives with double bonds that are located at non-native positions, such as at the ω-3, ω-5, ω-6, ω-8, ω-9, ω-11, and ω-12 positions.

Additionally or alternatively, the recombinant proteobacterium further comprises one or more enzymes such as ferredoxin, ferredoxin reductase or flavodoxin reductase, 3-hydroxyacyl-ACP-dehydratase (FabZ), a thioesterase, a carboxylic acid reductase, an alcohol dehydrogenase, a phosphopantetheinyl transferase, an alcohol acetyl-CoA transferase, a ω-hydroxylase, an alcohol oxidase/dehydrogenase, a fatty acid metabolism regulator protein (fadR), an aldehyde hydrogenase, and/or a β-ketoacyl-ACP synthase. One or more of the preceding enzymes may be native to the recombinant proteobacterium or one or more of the preceding enzymes may be heterologous (e.g., non-native). For example, for additional 3-hydroxy-acyl-ACP dehydratase activity in a recombinant ΔFabA strain (e.g., where FabA has been deleted), various heterologous FabZ enzymes can be employed, preferably 3-hydroxy-acyl-ACP dehydratases having activity to dehydrate all 3-hydroxy-acyl-ACP chain lengths (C4 to C18), such as FabZ from Acinetobacter baylyi (UniProtKB-Q6FCG4; SEQ ID NO:16), FabZ from Clostridium acetobutylicum (UniProtKB-Q97DA9; SEQ ID NO:27) or FabZ from Synechococcus elongatus (UniProtKB-Q31PQ9; SEQ ID NO:28).

In another embodiment, in addition to having native dual 3-hydroxy acyl-ACP dehydratase/isomerase (FabA) deleted, the recombinant proteobacterium may be further engineered to comprise a heterologous dual 3-hydroxy acyl-ACP dehydratase/isomerase (FabA), for example by the methods reported in WO 2020/047088, which as stated above, is incorporated herein by reference in its entirety.

In some embodiments, the recombinant proteobacterium produces at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99% of the total mUFAs or derivatives thereof. In a further embodiment, the recombinant proteobacterium may produce greater than 90% non-native mUFA or derivatives thereof, such as ω-5 unsaturated fatty acids or derivatives thereof. In particular, the mUFA or derivative thereof may be one or more of z9-tetradecenoic acid, z11-hexadecenoic acid, and z13-octadecenoic acid. Alternatively, the mUFA or derivative thereof may be one or more of z9-tetradecenol, z11-hexadecenol, z13-octadecenol, z7-tetradecenol, z9-hexadecenol and z11-octadecenol.

In a particular embodiment, the recombinant proteobacterium comprises a heterologous acyl-ACP desaturase, a heterologous acyl-ACP thioesterase, and a heterologous ferredoxin, wherein the native dual 3-hydroxy acyl-ACP dehydratase/isomerase (FabA) is deleted. In another embodiment, the recombinant proteobacterium overexpresses endogenous flavodoxin reductase or ferredoxin reductase (e.g., with a robust constitutive promoter) or expresses heterologous ferredoxin reductase or heterologous flavodoxin reductase.

In another particular embodiment, the recombinant proteobacterium comprises a heterologous acyl-ACP desaturase, a heterologous acyl-ACP thioesterase, and a heterologous 3-hydroxy acyl-ACP-dehydratase (FabZ), and optionally a heterologous ferredoxin and flavodoxin reductase or ferredoxin reductase, wherein the native dual 3-hydroxy acyl-ACP dehydratase/isomerase (FabA) is deleted.

In another particular embodiment, the recombinant proteobacterium comprises an increased level of FadR polypeptide expression. In this embodiment, the increased FadR expression may be native FadR, heterologous FadR, endogenous FadR, or heterologous and endogenous FadR. In another embodiment, the recombinant proteobacterium comprises an increased level of DNA binding activity of FadR as compared to a corresponding wildtype proteobacterium wherein the FadR is present at levels normal under natural conditions.

In another particular embodiment, the recombinant proteobacterium comprises a heterologous acyl-ACP desaturase, a heterologous acyl-ACP thioesterase, and a heterologous carboxylic acid reductase, and optionally a heterologous ferredoxin, heterologous flavodoxin reductase or ferredoxin reductase, and/or a heterologous 3-hydroxy acyl-ACP-dehydratase (FabZ), wherein the native dual 3-hydroxy acyl-ACP dehydratase/isomerase (FabA) is deleted. In a further embodiment, this recombinant proteobacterium produces an unsaturated fatty aldehyde or an unsaturated fatty alcohol. The unsaturated fatty aldehyde or unsaturated fatty alcohol may be z9-tetradecenal, z11-hexadecenal, z13-octadececanal, z9-tetradecenol, z11-hexadecenol, z13-octadececanol, and a combination thereof.

In another particular embodiment, the recombinant proteobacterium comprises a heterologous acyl-ACP desaturase, a heterologous acyl-ACP thioesterase, a heterologous carboxylic acid reductase and a heterologous alcohol dehydrogenase, and optionally one or more of a heterologous ferredoxin, a heterologous flavodoxin reductase or ferredoxin reductase, and a heterologous 3-hydroxy acyl-ACP-dehydratase (FabZ), wherein the native dual 3-hydroxy acyl-ACP dehydratase/isomerase (FabA) is deleted.

In another particular embodiment, the recombinant proteobacterium comprises a heterologous acyl-ACP desaturase, a heterologous acyl-ACP thioesterase, a heterologous carboxylic acid reductase, and a heterologous phosphopantetheinyl transferase, and optionally one or more of a heterologous ferredoxin, a heterologous flavodoxin reductase or ferredoxin reductase, a heterologous 3-hydroxy acyl-ACP-dehydratase (FabZ), and a heterologous alcohol dehydrogenase wherein the native dual 3-hydroxy acyl-ACP dehydratase/isomerase (FabA) is deleted.

In another particular embodiment, the recombinant proteobacterium comprises a heterologous acyl-ACP desaturase, a heterologous acyl-ACP thioesterase, a heterologous carboxylic acid reductase, and a heterologous alcohol acetyl-CoA transferase, and optionally one or more of a heterologous ferredoxin, a heterologous flavodoxin reductase or ferredoxin reductase, a heterologous 3-hydroxy acyl-ACP-dehydratase (FabZ), a heterologous alcohol dehydrogenase, and a heterologous phosphopantetheinyl transferase, wherein the native dual 3-hydroxy acyl-ACP dehydratase/isomerase (FabA) is deleted. In a particular embodiment, the recombinant proteobacterium comprises a heterologous acyl-ACP desaturase, a heterologous acyl-ACP thioesterase, a carboxylic acid reductase, and a heterologous alcohol acetyl-CoA transferase. In a further embodiment, this recombinant proteobacterium produces an unsaturated fatty alcohol acetate. The unsaturated fatty alcohol acetate may be consisting of z9-tetradecenyl acetate, z11-hexadecenyl acetate, z13-octadecenyl acetate, or a combination thereof.

In another particular embodiment, the recombinant proteobacterium comprises a heterologous acyl-ACP desaturase, a heterologous acyl-ACP thioesterase, and a heterologous ω-hydroxylase, and optionally one or more of a heterologous ferredoxin, a heterologous flavodoxin reductase or ferredoxin reductase, a heterologous 3-hydroxy acyl-ACP-dehydratase (FabZ), a heterologous carboxylic acid reductase, a heterologous alcohol dehydrogenase, a heterologous phosphopantetheinyl transferase, and a heterologous alcohol acetyl-CoA transferase, wherein the native dual 3-hydroxy acyl-ACP dehydratase/isomerase (FabA) is deleted. In a further embodiment, this recombinant proteobacterium produces an unsaturated ω-hydroxy fatty acid. The unsaturated ω-hydroxy fatty acid may be (z9)14-hydroxy-tetradecenoic acid, (z11)16-hydroxy-hexadecenoic acid, (z13)18-hydroxy-octadecenoic acid, or a combination thereof.

In another particular embodiment, the recombinant proteobacterium comprises a heterologous acyl-ACP desaturase, a heterologous acyl-ACP thioesterase, and a heterologous alcohol oxidase/dehydrogenase and optionally one or more of a heterologous ferredoxin, a heterologous 3-hydroxy acyl-ACP-dehydratase (FabZ), a heterologous carboxylic acid reductase, a heterologous alcohol dehydrogenase, a heterologous phosphopantetheinyl transferase, a heterologous alcohol acetyl-CoA transferase, and a heterologous ω-hydroxylase, wherein the native dual 3-hydroxy acyl-ACP dehydratase/isomerase (FabA) is deleted.

In another particular embodiment, the recombinant proteobacterium comprises a heterologous acyl-ACP desaturase, a heterologous acyl-ACP thioesterase, and a heterologous aldehyde dehydrogenase, and optionally one or more of a heterologous ferredoxin, a heterologous 3-hydroxy acyl-ACP-dehydratase (FabZ), a heterologous carboxylic acid reductase, a heterologous alcohol dehydrogenase, a heterologous phosphopantetheinyl transferase, a heterologous alcohol acetyl-CoA transferase, a heterologous ω-hydroxylase, and a heterologous alcohol oxidase/dehydrogenase, wherein the native dual 3-hydroxy acyl-ACP dehydratase/isomerase (FabA) is deleted. In a further embodiment, this recombinant proteobacterium produces an unsaturated α/ω-dicarboxylic acid. The unsaturated α/ω-dicarboxylic acid may be (z5)1,14-tetradecenedioic acid, (z5)1,16-hexadecenedioic acid, (z5)1,18-octadecenedioic acid, or a combination thereof.

In a specific embodiment, the recombinant proteobacterium comprises a heterologous acyl-ACP desaturase, a heterologous acyl-ACP thioesterase, a heterologous 3-hydroxy acyl-ACP-dehydratase (FabZ), a heterologous ferredoxin, and a heterologous flavodoxin reductase or ferredoxin reductase, and wherein the native dual 3-hydroxy acyl-ACP dehydratase/isomerase (FabA) is deleted.

In another particular embodiment, the recombinant proteobacterium produces non-native mUFAs and a heterologous acyl-ACP desaturase, a heterologous ferredoxin, a heterologous flavodoxin reductase or a heterologous ferredoxin reductase, a heterologous carboxylic acid reductase, and a heterologous 3-hydroxy-acyl-ACP dehydratase, wherein the native dual 3-hydroxy acyl-ACP dehydratase/isomerase (FabA) is deleted or attenuated. Additionally, the recombinant proteobacterium may comprise a heterologous acetyl-CoA transferase.

In another particular embodiment, the recombinant proteobacterium produces non-native mUFAs and comprises a heterologous acyl-ACP desaturase, a heterologous ferredoxin, a heterologous flavodoxin reductase or a heterologous ferredoxin reductase, a heterologous acyl-ACP reductase and a heterologous alcohol dehydrogenase, wherein the native dual 3-hydroxy acyl-ACP dehydratase/isomerase (FabA) is deleted or attenuated.

In another particular embodiment, the recombinant proteobacterium produces non-native mUFAs and comprises a heterologous acyl-ACP desaturase, a heterologous ferredoxin, a heterologous flavodoxin reductase or a heterologous ferredoxin reductase, a heterologous ω-hydroxylase, and a heterologous 3-hydroxy-acyl-ACP dehydratase, wherein the native dual 3-hydroxy acyl-ACP dehydratase/isomerase (FabA) is deleted or attenuated. Additionally, the recombinant proteobacterium may comprise a heterologous alcohol dehydrogenase and/or a heterologous aldehyde dehydrogenase.

In a specific embodiment, the recombinant proteobacterium produces non-native mUFAs and comprises a heterologous acyl-ACP desaturase, a heterologous acyl-ACP thioesterase, a heterologous ferredoxin, and a heterologous β-ketoacyl-ACP-synthase, and wherein the native dual 3-hydroxy acyl-ACP dehydratase/isomerase (FabA) is deleted. Additionally or alternatively, the recombinant proteobacterium has an increased level of expression of endogenous β-ketoacyl-ACP-synthase as compared to a corresponding wild type proteobacterium. For example, non-native ω5-unsaturated fatty acid derivatives that may be produced include z9-tetradecenoic acid, z11-hexadecenoic acid and/or z13-octadecenoic acid.

In a specific embodiment, the recombinant proteobacterium produces unsaturated fatty alcohols and comprises a heterologous acyl-ACP desaturase, a heterologous acyl-ACP thioesterase, a heterologous carboxylic acid reductase, a heterologous alcohol dehydrogenase, a heterologous phosphopantetheinyl transferase, a heterologous ferredoxin, and a heterologous β-ketoacyl-ACP-synthase, and wherein the native dual 3-hydroxy acyl-ACP dehydratase/isomerase (FabA) is deleted. For example, the fatty alcohols z9-tetradecenol, z11-hexadecenol and/or z13-octadecenol may be produced.

In a specific embodiment, the recombinant proteobacterium produces ω-hydroxy fatty acids and comprises a heterologous acyl-ACP desaturase, a heterologous acyl-ACP thioesterase, a heterologous ferredoxin, and a heterologous β-ketoacyl-ACP-synthase, and wherein the native dual 3-hydroxy acyl-ACP dehydratase/isomerase (FabA) is deleted. For example, the ω-hydroxy fatty acids (z9)14-hydroxy-tetradecenoic acid, (z11)16-hydroxy-hexadecenoic acid and/or (z13)18-hydroxy-octadecenoic acid may be produced.

In a specific embodiment, the recombinant proteobacterium produces fatty alcohol acetate esters and comprises a heterologous acyl-ACP desaturase, a heterologous acyl-ACP thioesterase, a heterologous carboxylic acid reductase, a heterologous ferredoxin, and a heterologous acetyl-CoA transferase, and wherein the native dual 3-hydroxy acyl-ACP dehydratase/isomerase is (FabA) deleted. For example, the fatty alcohol acetates z9-tetradecenyl acetate, z11-hexadecenyl acetate and/or z13-octadecenyl acetate may be produced.

In a specific embodiment, the recombinant proteobacterium produces fatty aldehydes and comprises a heterologous acyl-ACP desaturase, a heterologous acyl-ACP thioesterase, a carboxylic acid reductase, a heterologous ferredoxin and has one more deletions in alcohol dehydrogenase and/or aldehyde reductase genes. For example, the fatty aldehydes z11-hexadecenal, z9-tetradecenal and/or z13-octadecenal) may be produced.

In a specific embodiment, the recombinant proteobacterium produces α/ω-dicarboxylic acids and comprises a heterologous acyl-ACP desaturase, a heterologous acyl-ACP thioesterase, a heterologous alcohol dehydrogenase, a heterologous ω-hydroxylase, and a heterologous aldehyde dehydrogenase, and wherein the native dual 3-hydroxy acyl-ACP dehydratase/isomerase (FabA) is deleted. For example, the α/ω-dicarboxylic acids (z5)1,14-tetradecendioic acid, (z5)1,16-hexadecendioic acid and/or (z5)1,18-octadecendioic acid may be produced.

In a specific embodiment, the recombinant proteobacterium produces non-native monounsaturated free fatty acids and comprises a heterologous acyl-ACP desaturase, a heterologous ferredoxin, a heterologous flavodoxin reductase or a heterologous ferredoxin reductase, a heterologous carboxylic acid reductase, and a heterologous 3-hydroxy-acyl-ACP dehydratase, and wherein the native dual 3-hydroxy acyl-ACP dehydratase/isomerase (FabA) is deleted or attenuated.

In a specific embodiment, the recombinant proteobacterium produces non-native monounsaturated fatty aldehyde and/or fatty alcohol and comprises a heterologous acyl-ACP desaturase, a heterologous ferredoxin, a heterologous flavodoxin reductase or a heterologous ferredoxin reductase, a heterologous acyl-ACP reductase and a heterologous alcohol dehydrogenase, and wherein the native dual 3-hydroxy acyl-ACP dehydratase/isomerase (FabA) is deleted or attenuated

In a specific embodiment, the recombinant proteobacterium produces ω-hydroxy fatty acids and comprises a heterologous acyl-ACP desaturase, a heterologous ferredoxin, a heterologous flavodoxin reductase or a heterologous ferredoxin reductase, a heterologous ω-hydroxylase, and a heterologous 3-hydroxy-acyl-ACP dehydratase, and wherein the native dual 3-hydroxy acyl-ACP dehydratase/isomerase (FabA) is deleted or attenuated.

In some exemplary embodiments, the host cell (e.g., a recombinant proteobacterium) may further comprise genetic manipulations and alterations to enhance or otherwise fine tune the production of non-native mUFAs or derivatives thereof. The optional genetic manipulations can be used interchangeably from one host cell to another, depending on what other heterologous enzymes and what native enzymatic pathways are present in the host cell. Some optional genetic manipulations include one or more of the following:

FadE (Acyl-CoA dehydrogenase) catalyzes the first step in fatty acid utilization/degradation (β-oxidation cycle) which is the oxidation of acyl-CoA to 2-enoyl-CoA (see e.g., Campbell, J. W. and Cronan, J. E. Jr (2002) J. Bacteriol. 184(13): 3759-3764, Lennen, R. M. and Pfleger, B. F (2012) Trends Biotechnol. 30(12):659-667). Since FadE initiates the β-oxidation cycle, when E. coli lacks FadE, it cannot grow on fatty acids as a carbon source (see e.g., Campbell, J. W. and Cronan supra). The same effect can be achieved by attenuating other enzymes from the β-oxidation cycle, e.g., FadA, which is a 3-ketoacyl-CoA thiolase, or FadB, which is a dual 3-hydroxyacyl-CoA-dehydrogenase/dehydratase.

However, when E. coli is grown on a carbon source other than fatty acids e.g., grown on sugar, acetate, etc., FadE attenuation is optional because under such conditions FadE expression is repressed by FadR. Therefore, when cells are grown on a simple carbon source such as e.g., glucose, the FadE gene product is already attenuated. Accordingly, when grown on a carbon source other than fatty acids, a FadE mutation/deletion is optional.

In some embodiments, the fatty acid biosynthetic pathway in the production host uses the precursors acetyl-CoA and malonyl-CoA. E. coli or other host organisms engineered to overproduce these components can serve as the starting point for subsequent genetic engineering steps to provide the specific output product (such as, fatty acids, fatty esters, hydrocarbons, fatty alcohols). Several different modifications can be made, either in combination or individually, to the host strain to obtain increased acetyl-CoA/malonyl-CoA/fatty acid and fatty acid derivative production. See, for example, U.S. Patent Application Publication 2010/0199548, which is incorporated herein by reference in its entirety.

Other exemplary modifications of a host cell include, e.g., overexpression of non-native and/or native and/or variants of genes involved in the synthesis of acyl-ACP. In general, increasing acyl-ACP synthesis increases the amount of acyl-ACP, which is the substrate of thioesterases, ester synthases and acyl-ACP reductases. Exemplary enzymes that increase acyl-ACP production include e.g., enzymes that make up the “fatty acid synthase” (FAS). As is known in the art (see e.g., U.S. 2010/0199548) FAS enzymes are a group of enzymes that catalyze the initiation and elongation of acyl chains. The acyl carrier protein (ACP) along with the enzymes in the FAS pathway control the length, degree of saturation, and branching of the fatty acids produced. FAS pathway enzymes FabA, FabB, FabZ, and FadR are discussed above. Additional enzymes that comprise FAS include e.g., AccABCD, FabD, FabH, FabG, FabI, FabK, FabL, FabM, FabQ, FabV, FabX, and FabF. Depending upon the desired product one or more of these genes can be attenuated or over-expressed.

In some embodiments, a host strain may overexpress one or more of the FAS genes. Exemplary FAS genes that may be overexpressed include e.g., FadR from Escherichia coli (NP_415705.1), FabB from Escherichia coli (P0A953) or FabZ from Acinetobacter baylyi (Q6FCG4). In some exemplary embodiments, the overexpression of one or more of these genes, which code for enzymes and regulators in fatty acid biosynthesis, serves to further increase the titer of fatty-acid derivative compounds under particular culture conditions.

In another embodiment, the recombinant proteobacterium is produced through adaptive evolution of a wild-type proteobacterium or a different recombinant proteobacterium. In a particular embodiment, strain sAS.561 underwent adaptive evolution to obtain sAS.571. In another embodiment, the evolved proteobacterium contains one or more mutations. In a particular embodiment, the mutation is may be a C4T mutation in the promoter region of the yibL gene, which may up or down regulate the expression of the YibL gene product; a 18 bp in-frame insertion into the ptsI gene, which may effect PtsI expression and/or activity; and an IS1 mediated 65 bp insertion into the upstream region of the uof gene, which has been implicated in the translation of Fur, the major regulator of gene expression in response to iron (fur regulon). In a still further embodiment, the evolved proteobacterium comprises each of the C→T mutation in the promoter region of the yibL gene, an 18 bp in-frame insertion into the ptsI gene, and an IS1 mediated 65 bp insertion into the upstream region of the uof gene.

### III. Vectors

Also described herein are vectors comprising a nucleotide sequence that encodes one or more heterologous enzymes, including one or more of a heterologous acyl-ACP desaturase, a heterologous acyl-ACP thioesterase, a heterologous ferredoxin, a heterologous 3-hydroxyacyl-ACP-dehydratase (FabZ), a heterologous carboxylic acid reductase, a heterologous alcohol dehydrogenase, a heterologous phosphopantetheinyl transferase, a heterologous alcohol acetyl-CoA transferase, a heterologous (o-hydroxylase, a heterologous alcohol oxidase/dehydrogenase, a heterologous fatty acid metabolism regulator protein (fadR), a heterologous aldehyde hydrogenase, and a heterologous β-ketoacyl-ACP synthase. For example a vector comprising a nucleotide sequence encoding a Δ9-tetradecanoyl-acyl-ACP desaturase having at least 85%, at least 90%, at least 95% or 100% sequence identity to SEQ ID NO: 12 may be constructed by methods well known in the art.

The nucleotide sequence encoding the one or more enzymes, such as Δ9-tetradecanoyl-acyl-ACP desaturase, may be operably linked to one or more heterologous regulatory elements. Where the vector comprises a nucleotide sequence encoding more than one of the enzymes recited above, the vector may comprise a single heterologous regulatory element that directs expression of both enzymes or multiple heterologous regulatory elements that independently directs expression of each of the enzymes encoded by the vector.

As noted above, a polynucleotide or polypeptide can be overexpressed using methods well known in the art. In some embodiments, overexpression of a polypeptide is achieved by the use of an exogenous regulatory element. The term “exogenous regulatory element” generally refers to a regulatory element originating outside of the host cell. However, in certain embodiments, the term “exogenous regulatory element” can refer to a regulatory element derived from the host cell whose function is replicated or usurped for the purpose of controlling the expression of an endogenous polypeptide. For example, if the host cell is an E. coli cell, and the FadR polypeptide is encoded by an endogenous fadR gene, then expression of the endogenous fadR can be controlled by a promoter derived from another E. coli gene.

In some embodiments, the exogenous regulatory element is a chemical compound, such as a small molecule. As used herein, the term “small molecule” refers to a substance or compound having a molecular weight of less than about 1,000 g/mol.

In some embodiments, the exogenous regulatory element is an expression control sequence which is operably linked to the endogenous gene by recombinant integration into the genome of the host cell. In certain embodiments, the expression control sequence is integrated into a host cell chromosome by homologous recombination using methods well known in the art (e.g., Datsenko et al., Proc. Natl. Acad. Sci. U.S.A., 97(12): 6640-6645 (2000)).

In some embodiments, a vector described herein comprises a promoter operably linked to the polynucleotide sequence. In certain embodiments, the promoter is a developmentally-regulated promoter, an organelle-specific promoter, a tissue-specific promoter, an inducible promoter, a constitutive promoter, or a cell-specific promoter.

In some embodiments, a vector described herein comprises at least one sequence which may be (a) an expression control sequence (or regulatory element) operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech, Inc., Piscataway, NJ; Smith et al., Gene, 67: 31-40 (1988)), pMAL (New England Biolabs, Beverly, MA), and pRITS (Pharmacia Biotech, Inc., Piscataway, N.J.), which fuse glutathione S-transferase (GST), maltose E binding protein, or protein A, respectively, to the target recombinant polypeptide.

Suitable expression systems for both prokaryotic and eukaryotic cells are well known in the art; see, e.g., Sambrook et al., “Molecular Cloning: A Laboratory Manual,” second edition, Cold Spring Harbor Laboratory (1989). Examples of inducible, non-fusion E. coli expression vectors include pTrc (Amann et al., Gene, 69: 301-315 (1988)) and PET I1d (Studier et al., Gene Expression Technology: Methods in Enzymology 185, Academic Press, San Diego, CA, pp. 60-89 (1990)). In certain embodiments, a polynucleotide sequence of the invention is operably linked to a promoter derived from bacteriophage T5. Examples of vectors for expression in yeast include pYepSec1 (Baldari et al., EMBO J., 6: 229-234 (1987)), pMFa (Kurjan et al., Cell, 30: 933-943 (1982)), pJRY88 (Schultz et al., Gene, 54: 113-123 (1987)), pYES2 (Invitrogen Corp., San Diego, CA), and picZ (Invitrogen Corp., San Diego, CA). Baculovirus vectors available for expression of proteins in cultured insect cells (e.g., Sf9 cells) include, for example, the pAc series (Smith et al., Mol. Cell Biol., 3: 2156-2165 (1983)) and the pVL series (Lucklow et al., Virology, 170: 31-39 (1989)). Examples of mammalian expression vectors include pCDM8 (Seed, Nature, 329: 840 (1987)) and pMT2PC (Kaufinan et al., EMBO J., 6: 187-195 (1987)).

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by growth in the presence of an appropriate selection drug.

Similarly, for stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to an antibiotic) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by growth in the presence of an appropriate selection drug.

### IV. Methods of Producing Non-Native mUFAs or Derivatives Thereof, Cell Cultures and Fatty Acid Compositions

Methods of producing non-native mUFAs or derivatives thereof, cell cultures and fatty acid compositions are also described herein.

The recombinant proteobacterium described herein can be used to produce mUFAs or derivatives thereof, particularly ω-5 mUFAs or derivatives thereof. Thus, in one embodiment, a method is provided herein comprising culturing a recombinant proteobacterium comprising an acyl-ACP desaturase and an acyl-ACP thioesterase, wherein a native dual 3-hydroxy acyl-ACP dehydratase/isomerase (FabA) is deleted or attenuated in or on a suitable carbon source. As described above, the recombinant proteobacterium may further comprise one or more enzymes, such as a ferredoxin, flavodoxin reductase or ferredoxin reductase, a 3-hydroxyacyl-ACP-dehydratase (FabZ), a carboxylic acid reductase, an alcohol dehydrogenase, a phosphopantetheinyl transferase, an alcohol acetyl-CoA transferase, a ω-hydroxylase, an alcohol oxidase/dehydrogenase, a fatty acid metabolism regulator protein (fadR), an aldehyde hydrogenase, and a β-ketoacyl-ACP synthase. These enzymes may be native or heterologous, endogenous or exogenous. The recombinant proteobacterium may also comprise a heterologous dual 3-hydroxy acyl-ACP dehydratase/isomerase (FabA).

In general, non-native mUFAs and/or derivatives thereof are prepared by growing and/or fermenting the recombinant proteobacteria on or in suitable a carbon source. The recombinant proteobacteria are grown and/or fermented under appropriate conditions for a sufficient period of time to produce mUFAs and/or derivatives thereof. The carbon source may be culture media that comprises carbohydrates (e.g., monosaccharides, oligosaccharides, and polysaccharides), supplements (e.g., amino acids, antibiotics, polymers, acids, alcohols, aldehydes, ketones, peptides, and gases), and mineral salts. In a particular embodiment the carbon source is LB media or nitrogen (N)-mineral media with glucose as a carbon source.

Thus, also provided herein is a cell culture comprising the recombinant proteobacteria described herein and one or more mUFAs or derivatives thereof. Additionally provided herein is a fatty acid composition produced by the recombinant proteobacterium as described herein.

In some embodiments, the mUFA or derivative thereof is placed in a composition comprising the mUFA or derivative thereof, wherein the mUFA or derivative thereof is prepared by culturing and/or fermenting the recombinant proteobacterium. In some embodiments, the composition comprises one or more than one (e.g., two, three, four, five, or more) mUFA or derivative thereof.

Additionally, a composition is provided herein comprising more ω-5 unsaturated fatty acids or derivatives thereof than ω-7 unsaturated fatty acids derivatives thereof. The composition may be produced by the recombinant proteobacterium disclosed herein, and the compositions may comprise unsaturated fatty acids or other derivatives such as fatty acids, fatty acid esters, FAME, FAEE, FACE, fatty amines, fatty aldehydes, fatty alcohols, hydrocarbons, ketones, terminal olefins, internal olefin, 3-hydoxy fatty acid derivatives, bifunctional fatty acid derivatives, and unsaturated fatty acid derivatives as disclosed herein. For example, a composition may comprise at least about 5% more, at least about 10% more, at least about 15% more, at least about 20% more, at least about 25% more, at least about 30% more, at least about 35% more, at least about 40% more, at least about 45% more, at least about 50% more, at least about 55% more, at least about 60% more, at least about 65% more, at least about 70% more, at least about 75% more, at least about 80% more, at least about 85% more, at least about 90% more, at least about 95% more, or at least about 99% more ω-5 unsaturated fatty acids or derivatives thereof than ω-7 unsaturated fatty acids or derivatives thereof based on the total weight of the composition. Alternatively, the composition comprises ω-5 unsaturated fatty acids or derivatives thereof and no or substantially no ω-7 unsaturated fatty acids or derivative thereof (e.g., only trace amounts of ω-7 unsaturated fatty acids or derivatives thereof).

In another embodiment, a composition may comprise at least about 55%, at least about 60%, at least about 65%, at least about 70%, at least about 80%, at least about 85%, at least about 90%, at least about 95%, or at least about 99% ω-5 unsaturated fatty acids or derivatives thereof based on the total weight of the composition. Additionally, a composition may comprise about 50% or less, about 45% or less, about 40% or less, about 35% or less, about 30% or less, about 25% or less, about 20% or less, about 15% or less, about 10% or less, about 5% or less, about 4% or less, about 3% or less, about 2% or less, about 1% or less, or substantially no ω-7 unsaturated fatty acids or derivatives thereof based on the total weight of the composition.

Examples of mixture ratios of ω-5 unsaturated fatty acids or derivatives to ω-7 unsaturated fatty acids or derivatives thereof for the compositions provided herein include, but are not limited to, 55%/45%, 60%/40%, 65%/35%, 70%/30%, 75%/25%, 80%/20%, 85%/15%, 90%/10%, 95%/5%, 96%/4%, 97%/3%, 98%/2%, 99%/1%, wherein the first percentage value is the % of ω-5 unsaturated fatty acids or derivatives and the second percentage value is the % ω-7 unsaturated fatty acids or derivatives in the composition. Alternatively, the composition may comprise no or only trace amounts of the ω-7 unsaturated fatty acids.

In a particular example, the composition may comprise more ω-5 unsaturated fatty alcohols than ω-7 unsaturated fatty alcohols, e.g. about 90% of ω-5 unsaturated fatty alcohols and 10% ω-7 unsaturated fatty alcohols (e.g. 80% of z11-hexadecenol and 10% of z13-octadecenol and 10% of z9-hexadecenol).

In another particular example, the composition may comprise more ω-5 unsaturated fatty acids than ω-7 unsaturated fatty acids, e.g., about 95% of ω-5 unsaturated fatty acids, and 5% ω-7 unsaturated fatty acids. In another embodiment, the composition may comprise about 99%, or greater than 99% of ω-5 unsaturated fatty acids (e.g. z11-hexadecenoic acid and z9-tetradecenoic acid) and about 1% or less than 1% ω-7 unsaturated fatty acids. In still another embodiment, the composition may comprise essentially only ω-5 unsaturated fatty acids and no or only trace amounts of ω-7 unsaturated fatty acids.

### V. Uses

The recombinant proteobacterium described herein can be used for a variety of purposes. In particular, the recombinant proteobacterium may be used to produce insect pheromones or precursors thereof, and also for producing fragrances or precursors thereof.

In some embodiments, the mUFA or derivative thereof prepared by the cultured and/or fermented recombinant proteobacterium is used in a composition. In some embodiments, the mUFA or derivative thereof is a fermentation product of the recombinant proteobacterium. In other embodiments, the composition comprises one or more than one (e.g., two, three, four, five, or more) particular species of mUFA or derivative thereof. In a particular embodiment, the composition is an insect pheromone or precursor thereof, or a fragrance or a precursor thereof. In a particular embodiment, the mUFA or derivative thereof is purified.

In some embodiments, the mUFA or derivative thereof is prepared at a time and/or location that is different than when the composition is prepared. For example, the mUFA or derivative thereof may be produced by the recombinant proteobacterium in one location (e.g., a first facility, city, state, or country), transported to another location (e.g., a second facility, city, state, or country) and incorporated into the composition comprising the mUFA or derivative thereof.

In some embodiments, the mUFA or derivative thereof is purified prior to its use in the composition. The mUFA or derivative may be purified to a purity of at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, or at least about 99% free) from other components with which they are associated.

In some embodiments, the mUFA or derivatives thereof are insoluble or highly insoluble in water. In such cases, the mUFA or derivatives thereof are in a separate phase from the environment in which the recombinant proteobacteria reside (e.g., fermentation broth). In some embodiments, the mUFA or derivatives thereof are solid at room temperature. In another embodiment, the mUFA or derivatives thereof (e.g., alcohol derivatives) are liquid.

In a particular embodiment, purification of the mUFA or derivatives thereof involve isolating and recovering long chain alcohols. The product of the fermentation may be a heterogeneous mixture of a solid phase constituted by the biomass, the water phase of the fermentation broth, and a light liquid organic phase constituted by the secreted product. The separation of these phases can be achieved by centrifugation. Either disk-stack nozzle centrifuges or disk-stack ejector centrifuges can be used efficiently in different configurations. Typically, when using ejector machines, a 2-stage centrifugation scheme is recommended to achieve high clarity in the light phase (product) while minimizing losses. A nozzle centrifuge offers better clarification of the product in a single step centrifugation, but a more careful tuning of broth conditioning and centrifugation conditions may be required. In both cases, some conditioning of the fermentation broth (pH and temperature adjustments for example) may be desired to improve the overall performance of the centrifuges.

The light phase recovered is a crude organic phase composed by a mixture of fatty alcohols with a composition defined by the performance of the microorganism in use. This crude material can be further cleaned up by water-washing to remove any water-soluble compounds that might have been carried over, followed by water separation (centrifugation or gravity settling) and drying.

Additional purification steps may be required depending on the final product applications and specifications. These steps may include saponification, bleaching, and eventually distillation if high purity of a single chain length is required. All these are standard unit operations used regularly in the industry.

In another particular embodiment, purification of the mUFA or derivatives thereof involve isolating and recovering fatty acids. Purification of fatty acids differs from the separation of alcohols in that the fatty acids mixed with the biomass are both solids.

Two different approaches can be applied:

One approach includes recovery of the solid phase of biomass plus product via decanting centrifugation, followed by solvent extraction of the product from the biomass with an appropriate solvent (i.e., methanol or ethanol). The fatty acids dissolve in the solvent and the biomass is removed either by centrifugation or filtration. The recovery of the fatty acids is then completed by evaporating the solvent. Depending on the application the product can be further used as a solution in the solvent or as a solid. Other purification steps including distillation could be applied to meet final specifications.

Another approach includes recovery of the product via whole broth extraction with a water immiscible solvent. In this approach, the fermentation broth is contacted in either batch or continuous schemes with an appropriate solvent (i.e., butyl acetate, medium chain alcohols, or esters) to allow for the complete dissolution of the product in the solvent. The light organic solvent phase can be separated from the water phase in a similar way as those described for the recovery of the long chain alcohols. Once a clear solvent phase has been obtained, the final product is again recovered by solvent evaporation.

In another embodiment, the mUFA or derivative thereof prepared by the recombinant proteobacterium, or a composition comprising the mUFA or derivative thereof prepared by the recombinant proteobacterium is incorporated into a product. This product is made by combining, mixing, or otherwise using the mUFA or derivative thereof produced by the recombinant proteobacterium in combination with other or more additional components to prepare the product. The product may comprise one or more than one (e.g., two, three, four, five, or more) mUFA or derivative thereof prepared by the recombinant proteobacterium. In a particular embodiment, the product is a pheromone or precursor thereof, a fragrance or precursor thereof, or a nutritional supplement or precursor thereof.

### Insect Pheromones and Precursors Thereof in Crop Protection

Rising global demand for food combined with a growing concern about the environmental effects of overusing often toxic insecticides has magnified the need for more natural and sustainable means of crop protection such as insect pheromones, and precursors thereof, which are often mUFAs or derivatives thereof. The use of insect pheromones is a natural and non-toxic way of pest control and has gained attraction in agriculture in recent years. Insect pheromones can be used as lures in insect traps or sprayed on crops for insect mating disruption. However, so far synthetic pheromones are expensive to produce and are mostly synthesized from petroleum-derived feedstocks by sometimes complicated and/or low yielding chemical syntheses. Thus, in one embodiment, the recombinant proteobacterium described herein can be used for producing insect pheromones or pheromone precursors.

The insect pheromones or precursors thereof produced by the recombinant proteobacteria described herein may include derivatives of ω-5 unsaturated non-native fatty acids such as z9-tetradecenol, z11-hexadecenol, z13-octadecenol, z9-tetradecenal, z11-hexadecenal, z13-octadecenal, z9-tetradecenyl acetate, z11-hexadecenyl acetate, and z13-octadecenyl acetate. Specific examples of insect pheromones of important crop pests include, but are not limited to, z11-hexadecenal and/or z11-hexadecenyl acetate of the American cotton bollworm, the striped rice stem borer, and the diamondback moth (pests of cruciferous vegetables); z9-tetradecenal and/or z9-tetradecenyl acetate of the fall army worm (corn pests), the coddling moth, and the leafroller; and z13-octadecenal and/or z13-octadecenyl acetate of the Southwestern corn borer and the Asiatic rice borer. All these pheromones are derivatives of ω-5 mUFAs.

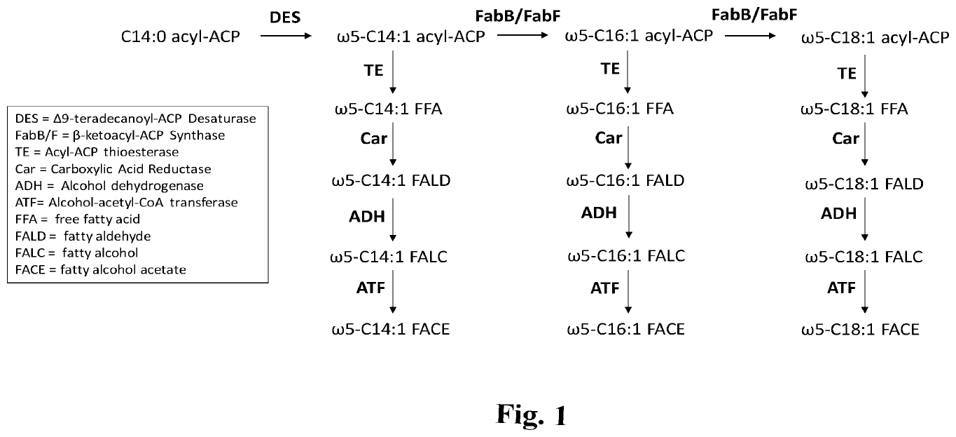

FIG. 1 summarizes the biochemical pathway converting bacterial acyl-ACPs to pheromones or pheromone precursors. A carboxylic acid reductase can efficiently convert the ω-5 non-native mUFA z9-tetradecenoic acid, z11-hexadecenoic acid, and z13-octadecenoic acid to the fatty aldehydes z9-tetradecenal, z11-hexadecenal, and z13-octadecenal, respectively. Particular carboxylic acid reductase or variants thereof are from Mycobacterium smegmatis. See WO 2010/062480 and WO 2013/152052, both of which are incorporated by reference in their entirety.

In one embodiment, the inventors have developed an efficient bacterial production system for ω-5 unsaturated pheromones or pheromone precursors that uses acyl-ACP intermediates and a novel biochemical pathway that includes a soluble Δ9-tetradecanoyl-ACP desaturase, an acyl-ACP thioesterase and a carboxylic acid reductase. The insect pheromone pathway does not contain any genes from insects and the recombinant proteobacterium has its FabA gene deleted. Therefore, only trace amounts of native ω-7 unsaturated fatty acid derivatives may be produced. For example, one recombinant strain produces ˜800 mg/L of z11-hexadecenol (˜80% of all fatty acid derivatives produced), but only trace amounts of z9-hexadecenol in a small-scale preparation. Thioesterase FatA from Arabidopsis thaliana has high specificity for acyl-ACPs with C16 chain length. Because proteobacteria (e.g., E. coli) have a type II fatty acid biosynthetic machinery, shorter chain acyl-ACPs are readily available in the cytoplasm for thioesterases to act upon. If FatA is replaced with e.g., an acyl-ACP thioesterase with high specificity for acyl-ACPs with C14 chain length (e.g., FatB from Cinnamomum camphorum or TE from Clostridium thermocellum), the strain may produce mainly z9-tetradecenol.

### Fragrances and Precursors Thereof

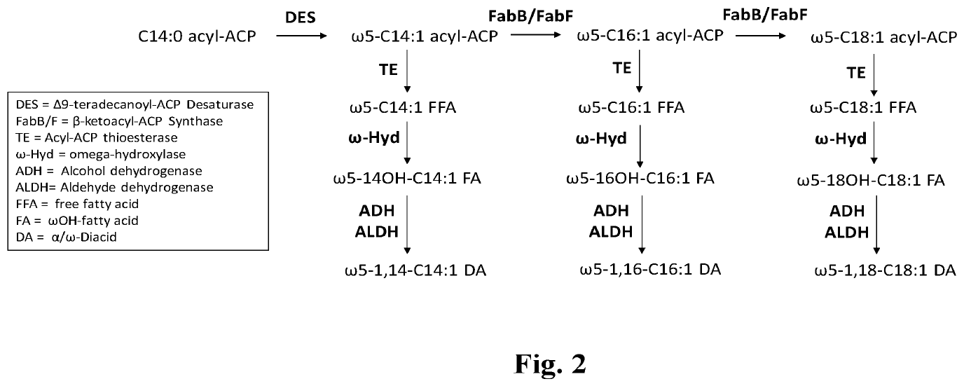

Additionally or alternatively, the recombinant proteobacterium described herein may be used for producing a fragrance or fragrance precursor. FIG. 2 summarizes the biochemical pathway converting bacterial acyl-ACPs to fragrance precursors. Sometimes these may be referred to as musk fragrances.

The inventors have discovered a novel pathway that utilizes an acyl-ACP desaturase (e.g., Δ9-tetradecanoyl-ACP desaturase), an acyl-ACP thioesterase, and a ω-hydroxylase to produce musk fragrance precursors in a recombinant recombinant proteobacterium such as E. coli. In a particular embodiment, the ω-hydroxylase is a hybrid-fusion P450 enzyme or a variant thereof as disclosed in WO 2014/201474 and/or WO 2017/106205, which are both herein incorporated by reference in their entirety. In a further particular embodiment, the ω-hydroxylase has at least 85%, at least 90%, at least 95% or 100% sequence identity to SEQ ID NO: 22.

The fragrance precursors which may be produced by the recombinant proteobacteria described herein include derivatives of ω-5 non-native mUFAs, such as (z9)14-hydroxy-tetradecenoic acid, (z11)16-hydroxy-hexadecenoic acid (z13)18-hydroxy-octadecenoic acid, (z5)1,14-tetradecendioic acid, (z5)1,16-hexadecendioic acid, and (z5)1,18-octadecendioic acid.

In Example 6 herein the production of ω5-non-native unsaturated ω-hydroxy fatty acid (z11)-16-hydroxy hexadecenoic acid is described. When this precursor is subjected to chemical lactonization (for methods see e.g., International Patent Application Publication WO 2015/157719 A9, which is incorporated herein by reference in its entirety), (12z)-1-Oxacycloheptadec-12-en-2-one is produced, which is struturally similar to the musk fragrances ambrettolide ((8z)-1-Oxacycloheptadec-8-en-2-one), cis-isoambrettolide ((10z)-1-Oxacycloheptadec-10-en-2-one) or isoambrettolide ((10e)-1-Oxacycloheptadec-10-en-2-one). (12z)-1-Oxacycloheptadec-12-en-2-one was reported to have a strong thibetolide-like odor similar to ambrette seeds (see U.S. Pat. No. 6,255,276), and is therefore an attractive novel musk fragrance. Hence, in one embodiment, the invention enables a cost-effective and steady supply of a natural precursor for this potential musk fragrance.

In Example 9 herein the production of the ω-5-non-native unsaturated α/ω-dicarboxylic acid (z5)1,16-hexadecenedioic acid is described. This precursor can be chemically converted to macrocyclic ketones similar to musk ketone fragrances (for methods see e.g., International Patent Application Publication WO 2015/157719, which is incorporated herein by reference in its entirety). For example, using decarboxylative chemistry (z5)1,16-hexadecenedioic acid can be converted to the 15-membered macrocyclic ketones (5z)-cyclopentadec-5-en-1-one or (4z)-cyclopentadec-4-en-1-one. The latter is known as the musk fragrance exaltenone. Using non-decarboxylative chemistry, (z5)1,16-hexadecenedioic acid can be converted to the 16-membered macrocyclic ketones (6z)-cyclohexadec-6-en-1-one or (5Z)-cyclohexadec-5-en-1-one. The latter is known as the musk fragrance ambrettone (also know as velvione). Therefore, in another embodiment, the invention enables a cost-effective and steady supply of the natural precursor for these commercial musk fragrances.

### EXAMPLES

The following examples are provided to further illustrate the invention disclosed herein but should not be construed as in any way limiting its scope.

### Example 1: Small Scale Fermentation

40 μL LB culture (from an LB culture growing in a 96 well plate) was used to inoculate 360 μL LB media, which was then incubated with shaking for approximately 4 hours at 32° C. 80 μL of the LB seed was used to inoculate 320 μL N-lim media (Table 1). After growing at 32° C. for 2 hours, the cultures were induced with isopropyl β-d-1-thiogalactopyranoside (IPTG) (final concentration 1 mM). The cultures were then incubated at 32° C. with shaking for 20 hours (unless otherwise noted), after which they were extracted with the extraction protocol detailed below.

TABLE 1
N-lim Media Formulation

| | | |
|---|---|---|
| 1 | x | 5x Salt Soln. with NH₄Cl |
| 1 | x | 1000x Trace Vitamins |
| 1 | mg/L | 10 mg/mL Thiamine |
| 1 | mM | 1M MgSO₄ |
| 0.1 | mM | 1M CaCl₂ |
| 40 | g/L | 500 g/L glucose |
| 1 | x | 1000x Trace minerals |
| 10 | mg/L | 10 g/L Fe Citrate |
| 100 | μg/mL | 100 mg/ml spectinomycin |
| 100 | mM | 2M BisTris (pH7.0) |
| 0.5 | mM | Aminolevulinic acid* |

*Aminolevulinic acid is only added when ω-hydroxy fatty acids or α/ω-diacids are expected products

### Free Fatty Acid Species Extraction and Analytical Protocol:

To each well to be extracted 30 μL of 12M HCl, followed by 400 μL of butyl acetate containing 500 mg/L 1-undecanol as an internal standard was added when fatty alcohols were quantified or 500 mg/L undecanoic acid as an internal standard were added when fatty acids or ω-hydroxy fatty acids were quantified. The 96 well plates were then heat-sealed and shaken for 30 minutes at 2000 rpm. After shaking, the plates were centrifuged for 10 minutes at 4500 rpm at room temperature to separate the aqueous and organic layers. 50 μL of the organic layer was transferred to a 96 well plate) and derivatized with 50 μL of TMS/BSTFA. The plate was subsequently heat sealed and stored at −20° C. until evaluated by either GC-FID or GC-MS.

Fatty acids or (w-hydroxy fatty acid analytics: The GC-MS parameters used to generate chromatograms and mass spectra for compounds identification were as follows:

TABLE 2

| | |
|---|---|
| Sample volume | 1 μL |
| Column | DB-1HT, 10 m × 250 μm × 0.1 μm |
| Initial temperature | 50° C. for 5 minutes |
| Final Temperature | 300° C., held for 5.24 minutes |
| Temperature increase rate | 25° C./minute |
| Total run time | 24 minutes |
| Column flow rate | 1.2 mL/min |
| Inlet temperature | 300° C. |
| Split ratio | 20:1 |
| Analyzing software | ChemStation E.02.01.1177 |

The mass spectrometry parameters are shown in Table 3.

TABLE 3

| | |
|---|---|
| Transfer line temperature | 300° C. |
| MS source temperature | 230° C. |
| MS Quad temperature | CombiPAL (CTC analytics) |

The GC-FID parameters used to quantify each compound are shown in Table 4:

TABLE 4

| | |
|---|---|
| Sample volume | 1 μL |
| Column | UFC Rtx-1, 5M × 0.1 mm × 0.1 μM |
| | (Thermo Fisher Ultrafast TRACE GC) |
| Initial oven temperature | 100° C. (0.2 minutes) |
| Final oven temperature | 320° C. (0.5 minutes) |
| Temperature increase rate | 100° C./min |
| Total run time | 2.5 minutes |
| Column flow rate | 0.5 mL/min |
| Inlet temperature | 300° C. |
| Flame ionization detector | 300° C. |
| temperature | |

Fatty alcohols analytics: The GC-FID parameters used to quantify each compound are shown in Table 5.

TABLE 5

| | |
|---|---|
| Sample volume | 1 μL |
| Column | UFC Rtx-1, 5M × 0.1 mm × 0.1 μM |
| | (Thermo Fisher Ultrafast TRACE GC) |
| Initial oven temperature | 100° C. (0.3 minutes) |
| Final oven temperature | 300° C. (0.05 minutes) |
| Temperature increase rate | 120° C./min |
| Total run time | 2.02 minutes |
| Column flow rate | 0.5 mL/min |
| Inlet temperature | 350° C. |
| Flame ionization detector | 300° C. |
| temperature | |

The protocols detailed above represents standard conditions, which may be modified as necessary.

### Cellular Fatty Acid Analysis:

Strains were grown over night in Luria-Bertani (LB) medium. The cellular fatty acids (i.e., fatty acids mainly from membrane phosholipids) were extracted and analyzed as follows: 10-20 mL of cultures were harvested and saponified at 70° C. for 1 hr using 1 mL of 15% (W/V) NaOH in 50% MeOH. The solutions were cooled down to room temperature and acidified by concentrated HCl to pH of 1-2. The acidic solution was then extracted with butyl acetate using a vortexer at 2500 rpm for 5 minutes. Extracts were centrifuged in an Eppendorf centrifuge at 15000 rpm for 5 minutes at room temperature. The organic layer (100 uL) was pipetted to a GC vial with insert, derivatized by adding 100 μL of N,O-bis(trimethylsilyl) trifluoroacetamide (BSTFA) and 1% trimethylchlorosilane (TMCS) and mixed using a vortexer for 30 seconds. The sample was then injected on GC-MS to generate chromatograms and mass spectra for compound identification. The GC-MS parameters were the same as in Table 2 and 3 above.

### Example 2: Identification of a Novel Δ9-Tetradecanoyl-ACP Desaturase

This example describes the discovery of a novel Δ9-tetradecanoyl-ACP desaturase.

A chloroplastic desaturase from Pelargonium xhortorum (DES_Pxho) (SEQ ID NOs: 1 and 2) (GenBank: AAC49421, UniProtKB-Q40879) is a Δ9-tetradecanoyl-ACP desaturase (see above). In order to identify another Δ9-tetradecanoyl-ACP and to compare with DES_Pxho, a comprehensive bioinformatics analysis of the family of plant acyl-ACP desaturases [EC 1.14.19.2] was conducted. Although the substrate specificity and regioselectivity of acyl-ACP desaturases cannot be predicted from their primary sequence, twenty putative “non-canonical” acyl-ACP desaturases with unknown substrate specificity and unknown regioselectivity were identified. These were not predicted to be canonical Δ9-stearoyl-ACP desaturases. Canonical Δ9-stearoyl-ACP desaturases are common and essential “housekeeping” desaturase enzymes in all plants and were not considered in this analysis.

The genes for the twenty unknown plant acyl-ACP desaturases were codon optimized for E. coli and cloned under control of an inducible Ptrc promoter into an expression vector (p15a replicon, kanamycin resistance marker). The twenty plasmids were transformed into an E. coli MG1655 derivative strain that contained an IPTG-inducible operon with a ferredoxin reductase (petH) (SEQ ID NOs: 3 and 4) (UniProtKB-B2IUI2) and a ferredoxin (petF) (SEQ ID NOs: 5 and 6) (UniProtKB-B2J405) from the cyanobacterium Nostoc punctiforme PCC73102. In addition, the strain harbored a second plasmid (SC101 replicon, spectinomycin resistance marker) containing, under control of the inducible Ptrc promoter, either a fatA thioesterase gene from Arabidopsis thaliana (SEQ ID NOs: 7 and 8) (UniProtKB-Q42561) (plasmid pAS.033) or a thioesterase gene from Clostridium thermocellum (SEQ ID NOs: 9 and 10) (UniProtKB-A3DJY9) (plasmid pAZ026).

Depending on which thioesterase plasmid was used, the strains allow identification of mainly C16 chain-length saturated and unsaturated free fatty acids released by FatA thioesterase (from plasmid pAS.033) or a mixture of C12-C18 chain-length saturated and unsaturated free fatty acids released by the Clostridium thioesterase (from plasmid pAZ026). The free fatty acids are secreted to the culture broth.

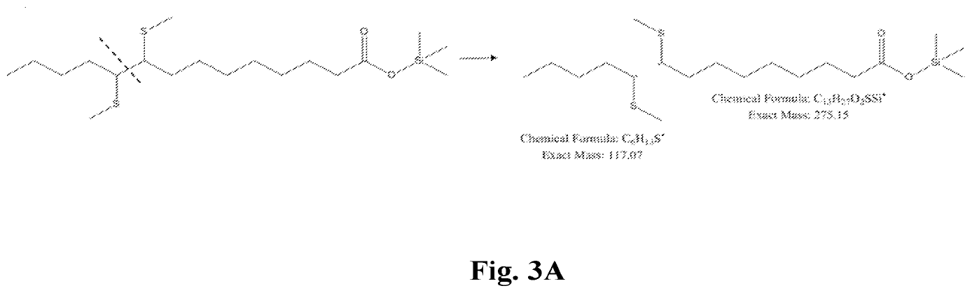

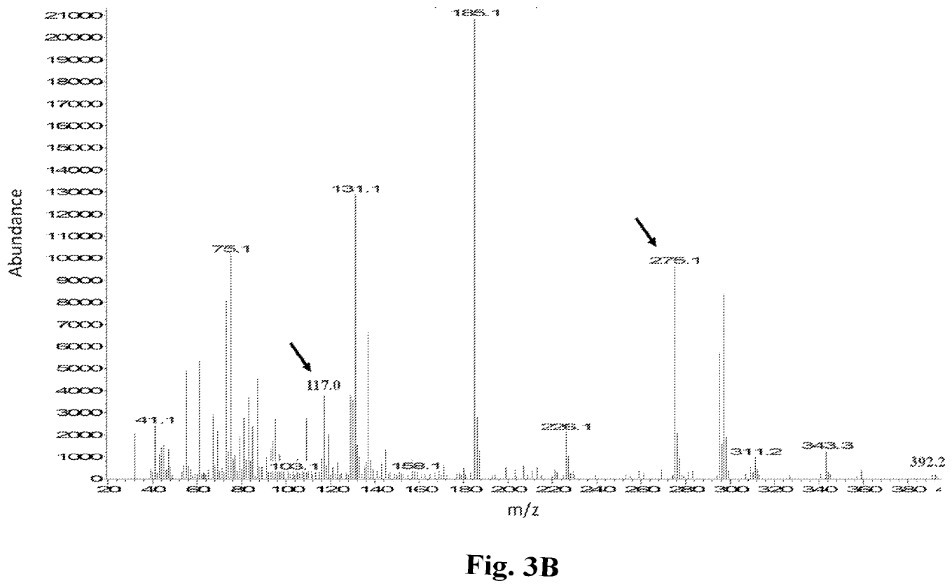

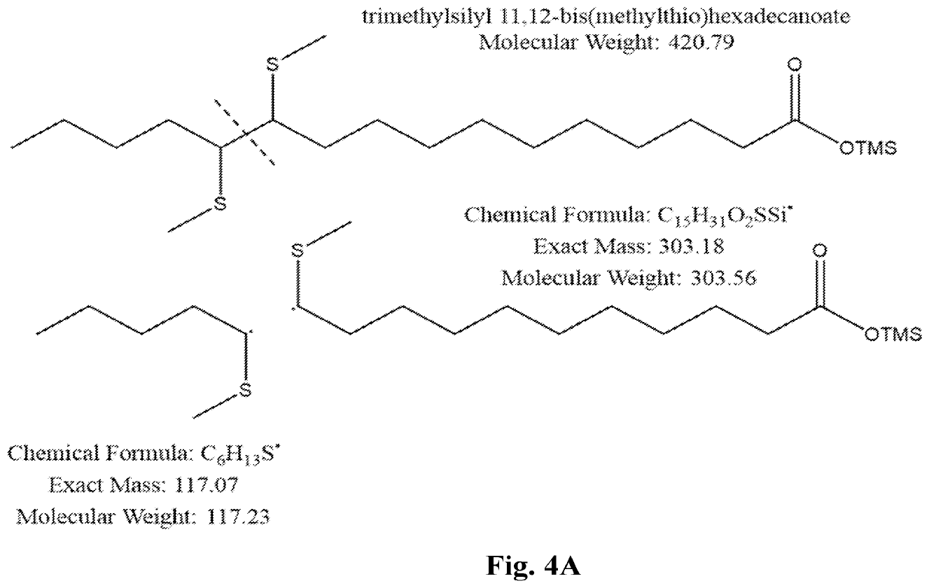

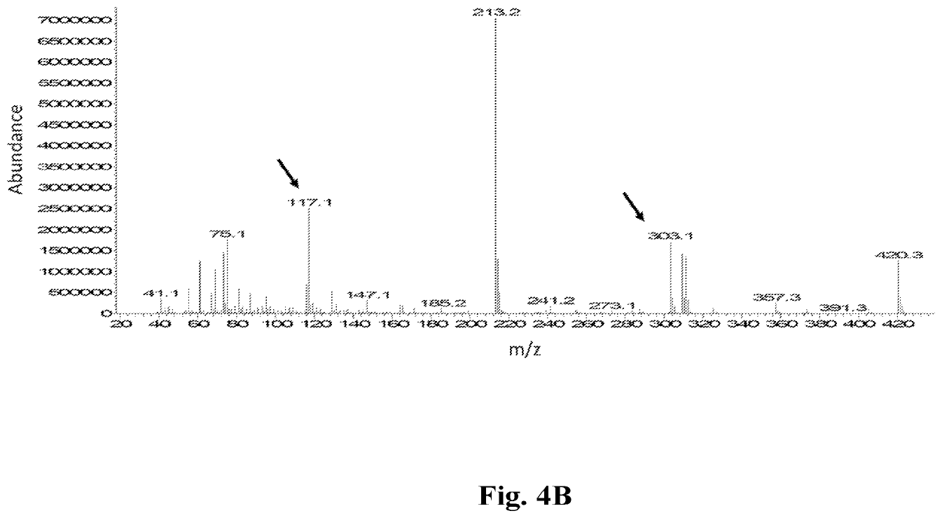

All strains were analyzed for their ability to produce non-native mUFAs (e.g., z9-tetradecenoic acid and z11-hexadecenoic acid) from glucose as described in Example 1. The peak identification for non-native fatty acids was performed using authentic standards for z9-tetradecenoic acid and z11-hexadecenoic acid, and the double bond position was confirmed by GC/MS of their dimethyl disulfide (DMDS) adducts (See Nichols et al. 1986, J. Microbiol. Methods 5: 49-55). FIGS. 3A, 3B, 4A and 4B show the fragmentation of z9-tetradecenoic acid and z11-hexadecenoic acid, respectively, under mass spectrometry and the resulting spectrum.

When compared with an empty vector (i.e., no desaturase) control strain, expression of one of the twenty “non-canonical,” unknown plant acyl-ACP desaturases gave rise to production of non-native mUFAs. Table 6 shows the composition of free fatty acids of the control strain and the strain harboring the novel desaturase F6HB23_VITV from Vitis vinifera (GenBank XP_002274652) (SEQ ID NOs: 11 and 12) and the Δ9-tetradecanoyl-ACP desaturase DES_Pxho.

With expression of FatA thioesterase, the only non-native unsaturated fatty acid produced in this strain was z8-hexadecenoic acid. The percentage of z11-hexadecenoic acid of the total fatty acids produced with F6HB23_VITV was 66% compared to 44% with DES_Pxho.

With expression of the thioesterase from Clostridium, the non-native mUFAs produced with DES_F6HB23_VITV were z9-tetradecenoic acid (22% of total fatty acids) and z11-hexadecenoic acid (16% of total fatty acids). This data indicates that z11-hexadecenoic acid is produced via elongation of Δ9-tetradecanoyl-ACP to Δ11-hexadecenoyl-ACP (by FabB and/or FabF enzymes) and that the novel desaturase acts on tetradecanoyl-ACP and not on hexadecanoyl-ACP.

This example shows that the desaturase F6HB23_VITV from Vitis vinfera is a novel Δ9-tetradecanoyl-ACP desaturase and when co-expressed with a thioesterase in a proteobacterium like E. coli gives rise to (ω-5 mUFAs such as z9-tetradecenoic acid and z11-hexadecenoic acid.

TABLE 6
Composition of free fatty acids produced in strains harboring
the novel desaturase F6HB23_VITV from Vitis vinifera

| | FAS | C8-C12 | z7C14:1 | z9C14:1 | C14:0 | z9C16:1 | z11C16:1 | C16:0 | C18 |
|---|---|---|---|---|---|---|---|---|---|
| | (mg/L) | (%) | (%) | (%) | (%) | (%) | (%) | (%) | (%) |
| | Plasmid pAS.033 with fatA thioesterase | | | | | | | | |
| Control | 1319 | 0 | 0 | 0 | 11 | 29 | 0 | 54 | 6 |
| Desaturase | 1223 | 0 | 0 | 0 | 12 | 12 | 66 | 10 | 0 |
| F6HB23_VITV | | | | | | | | | |
| Desaturase | 1455 | 0 | 0 | 0 | 5 | 34 | 44 | 15 | 2 |
| DES_Pxho | | | | | | | | | |
| | Plasmid pAZ026 with tesZ1 thioesterase | | | | | | | | |
| Control | 872 | 18 | 17 | 0 | 27 | 9 | 0 | 17 | 12 |
| Desaturase | 977 | 24 | 9 | 22 | 5 | 5 | 16 | 7 | 12 |
| F6HB23_VITV | | | | | | | | | |

### Example 3: Construction of a Recombinant E. coli Strain for the Production of Predominantly Non-Native ω-5 Unsaturated Fatty Acid Derivatives

This example describes the construction of a recombinant proteobacterium that contains predominantly non-native ω-5 unsaturated cellular fatty acids (i.e., within its cytoplasmic membrane) and only trace amounts of native ω-7 unsaturated cellular fatty acids. The strain has high flux through the fatty acid biosynthesis pathway and is suitable for high titer production of secreted ω-5 mUFAs or derivatives thereof when an acyl-ACP thioesterase or other fatty acid derivative enzymes (e.g., an ester synthase or an acyl-ACP reductase) are coexpressed.

The strain is a derivative of E. coli MG1655 and its genome was engineered as follows: the acyl-CoA dehydrogenase (fadE) gene was deleted and a variant of the transcriptional regulator FadR was overexpressed. Both modifications are optional. Next, an operon controlled by a constitutive PT5 promoter was integrated into the chromosome that contained the Δ9-tetradecanoyl-ACP desaturase gene (without the plastid targeting leader sequence) from P. xhortorum (DES_Pxho), and flavodoxin reductase (fpr) (SEQID NOs: 13 and 14) (UniProtKB-P28861) and ferredoxin (petF) from Nostoc punctiforme PCC73102 (SEQID NOs: 5 and 6), and subsequently the gene for a 3-hydroxy-acyl-ACP dehydratase (FabZ) (SEQID NOs 15 and 16) (UniProtKB-A0A0M1I0X8) from Acinetobacter baylyi controlled by a constitutive PT5 promoter was also integrated. Next, plasmid pIR.074 (p15a replicon, kanamycin resistance marker) containing the DES_Pxho gene under the control of an IPTG-inducible promoter, was transformed yielding strain AA.827.

The dual hydroxy-acyl-ACP dehydratase/isomerase FabA gene in AA.827 was deleted as follows: Plasmid pXC.006 (temperature sensitive SC101* replicon, ampicillin resistance marker) containing a temperature-sensitive FabA gene (FabA^{ts}, see Johnson and Greenberg 1975, J. Bacteriol. 122: 570-574) under the control of a constitutive promoter was transformed. Next, at the permissive temperature of 30° C., the chromosomal FabA gene was deleted, i.e., plasmid pXC.006 was maintained and FabA^{ts} enzyme was functional, and subsequently plasmid pXC.006 harboring FabA^{ts} was cured (i.e., removed) from the strain at an increased non-permissive temperature (42° C.), yielding strain sAS.561, which lacks FabA activity at all temperatures. Adaptive evolution was performed on strain sAS.561 and strain sAS.571 was obtained.

To determine the strain's ability to produce non-native ω-5 unsaturated mUFAs, the composition of the cellular fatty acids were determined as described in Example 1, and it was compared to a similar control strain but with intact FabA gene and without a desaturase gene (sZR.409). As shown in Table 7 the unsaturated fatty acids of strain AA.827, which contained the DES_Pxho gene but had FabA gene intact, consisted of ˜40% non-native ω-5unsaturated fatty acids. The unsaturated cellular fatty acid of strain sAS.571, which contained DES_Pxho and deletion of the FabA gene, consisted predominantly (˜95%) of non-native ω-5unsaturated fatty acids, and of those 86% were z11-hexadecenoic acid and 14% were z13-octadecenoic acid. Interestingly, the percent unsaturation of the cytoplasma membrane in sAS.571 was higher than for the control strain.

These results show that a Δ9-tetradecanoyl-ACP desaturase can functionally replace the 3-hydroxy-acyl-ACP dehydratase/isomerase FabA gene in a proteobacterium like E. coli and gives rise to predominantly ω-5 mUFAs such as z11-hexadecenoic acids and z13-octadecenoic acid when the FabA gene is deleted. As the recombinant strain described in this example has a high flux fatty acid biosynthesis pathway, it can be employed to produce and secrete non-native ω-5 mUFAs and derivatives thereof at high titer.

TABLE 7
Composition of cellular fatty acids
(CFAs) of recombinant E. coli strains

| | SFA ¹ | UFA² | | |
|---|---|---|---|---|
| | (% of | (% of | % ω-5 of | % ω-7 of |
| Strain | total) | total) | UFA | UFA² |
| SZR.409 | 36.1 | 63.9 | 1.5 | 98.5 |
| (control) | | | | |
| AA.827 | 34.2 | 64.9 | 40.5 | 59.5 |
| SAS.571 | 32.6 | 67.4 | 95.1 | 4.9 |

¹ saturated fatty acids: does not include 3-hydroxy tetradecanoic acid from outer membrane lipopolysaccharides
²unsaturated fatty acids: includes cyclopropane C17:0 fatty acid, which is derived from z9-hexadecenoic acid

### Example 4: Production of Non-Native ω5-Unsaturated Free Fatty Acids by a Recombinant E. coli Strain

This example shows that a recombinant ΔFabA E. coli strain expressing a Δ9-tetradecanoyl-ACP desaturase and a thioesterase predominantly produces non-native ω-5mUFAs and only trace amounts of native ω-7 unsaturated mUFAs.

Strain sAS.571 (prepared as in Example 3) was transformed with plasmid pKM.023 (SC101 replicon, spectinomycin resistance marker) containing an inducible Ptrc promoter and an operon with the fatA thioesterase gene from Arabidopsis thaliana and the native FabB β-ketoacyl-ACP synthase gene. The resulting strain was sAS.563. The control strain with intact FabA gene and without a desaturase gene was the same as the control strain in Example 3 transformed with plasmid pKM.023 (strain AA.804).

The strains were subjected to small scale fermentation and product analysis as described in Example 1. As shown in Table 8, strain sAS.563 produced over 2000 mg/L of free fatty acids at 32° C. and 37° C. with a high degree of unsaturation. All strains were evaluated for their ability to produce non-native mUFAs. The peak identification for z9-tetradecenoic acid and z11-hexadecenoic acid was performed by comparing their retention time and ion fragmentation pattern in GC/MS to that of authentic standards. The double bond position was confirmed by GC/MS of their dimethyl disulfide (DMDS) adducts as shown in FIGS. 3A-B and 4A-B. The peak identification for z13-octadecenoic acid was based on its expected retention time and ion fragmentation pattern relative to native z11-octadecenoic acid.

As shown in Table 8, ˜99% of the unsaturated fatty acid produced by sAS.563 had the double bond in the non-native ω-5 position with the majority being z11-hexadecenoic acid and smaller amounts of z9-tetradecenoic acid and z13-octadecenoic acid produced.

Interestingly, the E. coli control strain produced trace amounts of z11-hexadecenoic acid, which is not observed in the E. coli MG1655 wild-type strain. This may be attributed to the high fatty acid biosynthesis flux in the control strain allowing FabA to isomerize not only trans-2-decenoyl-ACP (native substrate) to cis-3-decenoyl-ACP (precursor to native ω-7 mUFA) but also to a minor extend trans-2-octenoyl-ACP (non-native substrate) to cis-3-octenoyl-ACP (precursor to non-native ω-5 mUFA). Similarly, trace amounts of z11-hexadecenoic acid were also found in the cellular fraction of the control strain without plasmid pKM.023 (see Example 3 and Table 7).

This example demonstrates that a recombinant ΔFabA E. coli strain expressing a Δ9-tetradecanoyl-ACP desaturase posseses a high flux fatty acid biosynthesis pathway and when co-expressed with a thioesterase produces non-native ω-5 unsaturated fatty acids at high titer and with only trace amounts of native ω-7 unsaturated fatty acids. Besides lesser amounts of z9-tetradecenoic acid and z13-octadecenoic acid, z11-hexadecenoic acid was the major non-native fatty acid produced.

TABLE 8
Composition and titer of free fatty acids (FFA) of recombinant E. coli strains

| | Total FFA | % UFA | % ω5 | z7-C14:1 | z9-C14:1 | z9-C16:1 | z11-C16:1 | z11-C18:1 | z13-C18:1 |
|---|---|---|---|---|---|---|---|---|---|
| strain | (mg/L) | of FFA | of UFA | (mg/L) | (mg/L) | (mg/L) | (mg/L) | (mg/L) | (mg/L) |
| AA.804 | 3558 | 70 | 1.3 | 0 | 0 | 2292 | 32 | 15 | 0 |
| (control) | | | | | | | | | |
| sAS.563 | 2094 | 77 | 99.2 | 0 | 133 | 12 | 1448 | 0 | 8 |
| (32C) | | | | | | | | | |
| sAS.563 | 2055 | 75 | 99.4 | 0 | 171 | 9 | 1363 | 0 | 6 |
| (37C) | | | | | | | | | |

### Example 5: Production of ω-5 Unsaturated Fatty Alcohols by Recombinant E. coli Strains

This example shows that a recombinant ΔFabA E. coli strain expressing a Δ9-tetradecanoyl-ACP desaturase, a thioesterase and a carboxylic acid reductase predominantly produces ω-5 unsaturated fatty alcohols (e.g., z11-hexadecenol and z9-tetradecenol) and only trace amounts of ω-7 unsaturated fatty alcohols (e.g., z9-hexadecenol).

In strain sAS.571 (prepared as in Example 3), the chromosomal entD gene was deregulated by replacing the native iron-regulated promoter of entD with a constitutive PT5 promoter. EntD is a promiscous phosphopantetheinyl transferase utilized in this strain to posttranslationally modify a carboxlic acid reductase. The resulting strain sAS.619 was transformed with plasmids pSVEN.250 and pSVEN.251, respectively, yielding strains sven.1236 and sven.1237. Both plasmids (SC101 replicon, spectinomycin resistance marker) contain two operons: the first operon contains under control of an inducible Ptrc promoter the fatA thioesterase gene from Arabidopsis thaliana and the FabB β-ketoacyl-ACP synthase gene; the second operon contains, under control of an inducible PT5 promoter, a carB carboxylic acid reductase gene from Mycobacterium smegmatis (SEQ ID NOs: 17 and 18) (UniProtKB-A0A653FCI4) and the alrA alcohol dehydrogenase gene from Acinetobacter baylyi (SEQ ID NOs: 19 and 20) (UniProtKB-Q6F6R9). The expression of the first operon was higher in plasmid pSVEN.250 than in pSVEN.251. A similar control strain with the intact FabA gene and without a desaturase (strain sOW.006) was used for this experiment.

The strains were subjected to small scale fermentation and product analysis as described in Example 1. Authentic standards for the non-native ω-5 unsaturated fatty alcohols were not available. Peak identification was based on their expected retention times and ion fragmentation patterns relative to native ω-7 unsaturated fatty alcohols. As shown in Table 9, both strains sven.1236 and sven.1237 produced a large amount of z11-hexadecenol and (˜800 mg/L) as well as z9-tetradecenol (41-123 mg/L), and both strains produced only trace amounts of the corresponding unsaturated fatty alcohols with the native ω-7 double bond (z9-hexadecenol). In contrast, the control strain produced a large amount of z9-hexadecenol (˜1400 mg/l) and only little z11-hexadecenol.

This example demonstrates that a recombinant ΔFabA E. coli strain expressing a Δ9-tetradecanoyl-ACP desaturase posseses a high flux fatty acid biosynthesis pathway and when co-expressed with a thioesterase and a carboxylic acid reductase produces ω5-unsaturated fatty alcohols at high titer and with only trace amounts of ω7-unsaturated fatty alcohols. Besides lesser amounts of z9-tetradecenol, z11-hexadecenol was the major fatty alcohol produced.

TABLE 9
Composition and titer of fatty alcohols (FALC) of recombinant E. coli strains

| | Total | Total | | % | | z7-C14:1 | z9-C14:1 | z9-C16:1 | z11-C16:1 |
|---|---|---|---|---|---|---|---|---|---|
| | FFA | FALC | % | [UFA + | % ω5 of | FALC | FALC | FALC | FALC |
| strain | (mg/L) | (mg/L) | FALC | UFALC] | UFALC | (mg/L) | (mg/L) | (mg/L) | (mg/L) |
| sOW.006 | 992 | 1598 | 61 | 90 | 3.2 | 7 | 0 | 1428 | 49 |
| (control) | | | | | | | | | |
| sven.1236 | 155 | 1123 | 88 | 79 | 99.6 | 0 | 123 | 3 | 795 |
| (32C) | | | | | | | | | |
| sven.1237 | 91 | 982 | 92 | 83 | 99.5 | 0 | 41 | 4 | 811 |
| (37C) | | | | | | | | | |

### Example 6: Production of ω-5 Unsaturated w-Hydroxy Fatty Acids by a Recombinant E. coli Strain

This example shows that a recombinant ΔFabA E. coli strain expressing a Δ9-tetradecanoyl-ACP desaturase, a thioesterase and a ω-hydroxylase predominantly produces ω-5 unsaturated ω-hydroxy fatty acids (e.g., (z11)-16-hydroxy hexadecenoic acid) and only trace amounts of ω-7 unsaturated ω-hydroxy fatty acids (e.g., (z9)-16-hydroxy hexadecenoic acid).

Strain sAS.571 (see Example 3) was transformed with plasmid pKM.010, which is a derivative of plasmid pKM.023 (SC101 replicon, spectinomycin resistance marker) (see Example 4) that, in addition to the fatA thioesterase and FabB β-ketoacyl-ACP synthase operon, contains under control of an inducible PT5 promoter a hybrid-fusion Cyp153-RhF ω-hydroxylase (SEQ ID NOs: 22 and 23). The resulting strain was sAS.583.

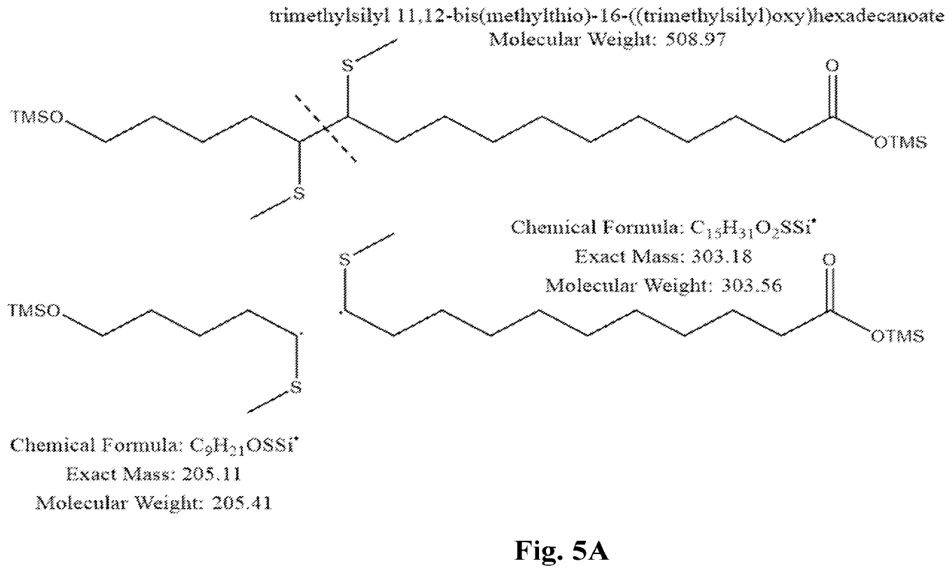

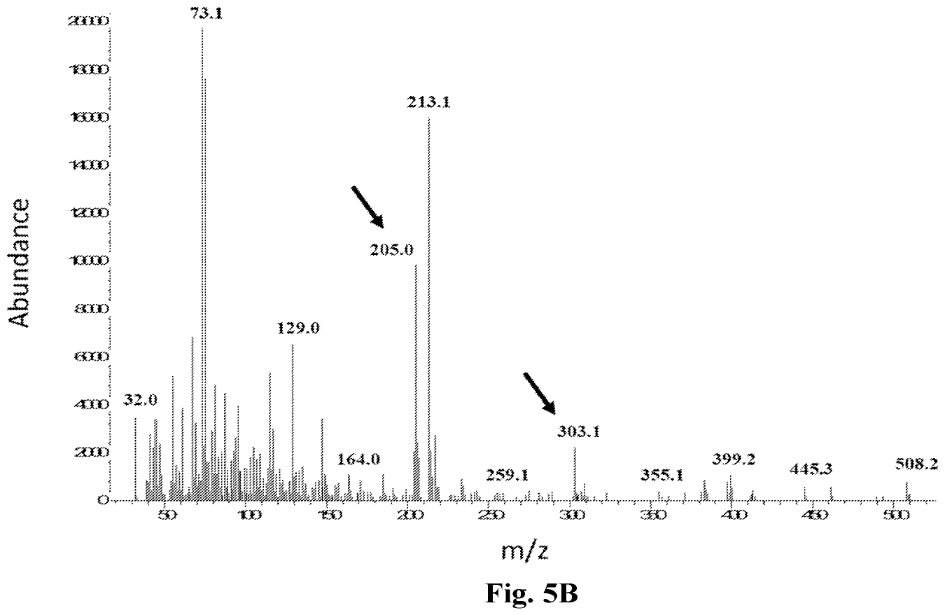

The strain was subjected to small scale fermentation and product analysis as described in Example 1. Authentic standards of ω-5 unsaturated ω-hydroxy fatty acids were not available. Peak identification was performed based on expected retention times and GC/MS fragmentation patterns relative to the native ω-7 unsaturated ω-hydroxy fatty acids. In addition, the double bond position of (z11)-16-hydroxy hexadecenoic was confirmed by GC/MS of its dimethyl disulfide (DMDS) adducts as shown in FIGS. 5A-5B.

Strain sAS.583 produced 390 mg/L of ω-hydroxy fatty acids and 40 mg/L of free fatty acids. The ω-hydroxy fatty acids consisted of 12 mg/L (z9)-14-hydroxy tetradecenoic acid, 41 mg/L 14-hydroxy tetradecanoic acid, 211 mg/L (z11)-16-hydroxy hexadecenoic acid and 126 mg/L 16-hydroxy hexadecanoic acid. The strain produced only trace amounts of (z9)-16-hydroxy hexadecenoic acid.

This example demonstrates that a recombinant ΔFabA E. coli strain expressing a Δ9-tetradecanoyl-ACP desaturase posseses a high flux fatty acid biosynthesis pathway and when co-expressed with a thioesterase and a ω-hydroxylase produces non-native ω-5 unsaturated ω-hydroxy fatty acids with only trace amounts of native ω-7 unsaturated ω-hydroxy fatty acids. Besides lesser amounts of (z9)-14-hydroxy tetradecenoic acid, (z11)-16-hydroxy hexadecenoic acid was the major ω-hydroxy fatty acid produced.

When (z11)-16-hydroxy hexadecenoic acid is subjected to chemical lactonization, (12z)-1-oxacycloheptadec-12-en-2-one is produced, which is struturally similar to musk fragrances such as ambrettolide ((8z)-1-oxacycloheptadec-8-en-2-one), cis-isoambrettolide ((10z)-1-oxacycloheptadec-10-en-2-one) or isoambrettolide ((10e)-1-oxacycloheptadec-10-en-2-one) (see e.g., International Patent Application Publication WO 2015/157719 A9).

### Example 7: Production of ω-5 Unsaturated Fatty Alcohol Acetate Esters by a Recombinant E. coli Strain

Recombinant ΔFabA E. coli strains expressing a Δ9-tetradecanoyl-ACP desaturase, a thioesterase, a carboxylic acid reductase and an acetyl-CoA transferase are expected to predominantly produce non-native ω-5 unsaturated fatty alcohol acetates (z11-hexadecenyl acetate, z9-tetradecenyl acetate, and z13-octadecenyl acetate) and only trace amounts of native ω-7 unsaturated fatty alcohol acetates (e.g., z9-hexadecenyl acetate).

A gene coding for an acetyl-CoA transferase [EC 2.3.1.84], e.g., Atf1 from Saccharomyces cerevisiae (SEQ ID NOs: 23 and 24) (UniProtKB-Q6XBT3), under the control of an inducible or constitutive promoter, is integrated into the chromosome of strain sAS.619 (as prepared in Example 5). The resulting strain is transformed with plasmids pSVEN.250 and pSVEN.251 (see Example 5), respectively, or with similar plasmids.

The strains are subjected to small scale fermentation and product analysis is carried out as described in Example 1. The recombinant proteobacteria are expected to produce non-native ω-5-unsaturated fatty alcohol acetates (z11-hexadecenyl acetate, z9-tetradecenyl acetate and/or z13-octadecenyl acetate) and only trace amounts of native ω7-unsaturated fatty alcohol acetates (e.g., z9-hexadecenyl acetate).

### Example 8: Production of ω-5 Unsaturated Fatty Aldehydes by a Recombinant E. coli Strain with Deleted Alcohol Dehydrogenase Genes

Recombinant ΔFabA E. coli strains with multiple deletions in alcohol dehydrogenase and/or aldehyde reductase genes, and expressing a Δ9-tetradecanoyl-ACP desaturase, a thioesterase and a carboxylic acid reductase are expected to predominantly produce non-native ω-5 unsaturated fatty aldehydes (e.g., z11-hexadecenal, z9-tetradecenal and z13-octadecenal) and only trace amounts of native ω-7 unsaturated fatty aldehydes (e.g., z9-hexadecenal).

Plasmids pSVEN.250 and pSVEN.251 (as prepared in Example 5) or similar plasmids are modified by deleting the AlrA gene from the second operon. Strain sAS.619 (see example 5) is modified by deleting or attenuating one or more of the yjgB, yahK, ybbO, YqhD, AdhP, EutG, YiaY, BetA, FucO, DkgA, YghA, GIdA or AdhE genes. The resulting strains are transformed with the modified plasmids and are subjected to small scale fermentation and product analysis as described in Example 1. The recombinant proteobacteria are expected to produce predominantly non-native ω-5 unsaturated fatty aldehydes (e.g., z11-hexadecenal, z9-tetradecenal and/or z13-octadecenal) and only trace amounts of native ω-7 unsaturated fatty aldehydes (e.g., z9-hexadecenal).

### Example 9: Production of ω-5 Unsaturated α/ω-Dicarboxylic Acids by a Recombinant E. coli Strain

Recombinant ΔFabA E. coli strain expressing a Δ9-tetradecanoyl-ACP desaturase, a thioesterase, a ω-hydroxylase and one or two heterologous dehydrogenases (an alcohol dehydrogenase and an aldehyde dehydrogenase) are expected to produce predominantly non-native ω-5 unsaturated α/ω-dicarboxylic acids (e.g., (z5)1,16-hexadecenedioic acid, (z5)1,14-tetradecenedioic acid and/or (z5)1,18-octadecenedioic acid acid) and only trace amounts of native ω-7 unsaturated α/ω-dicarboxylic acids (e.g., (z7)1,16-hexadecenedioic acid).

Two genes, coding for an alcohol dehydrogenase (e.g., UniProtKB-Q00593 from Pseudomonas oleovorans; SEQ ID NO:29) and an aldehyde dehydrogenase (e.g., UniProtKB-Q6FAS2 from Acinetobacter baylyi, SEQ ID NO:30), under the control of inducible or constitutive promoters are integrated into the chromosome of strain sAS.619 (as prepared in Example 5). The resulting strain is transformed with plasmid pKM.010 (see Example 5) or a similar plasmid and is subjected to small scale fermentation and product analysis as described in Example 1. The recombinant proteobacteria are expected to produce predominantly non-native ω-5 unsaturated α/ω-dicarboxylic acids (e.g., (z11)1,16-hexadecenedioic acid, (z5)1,14-tetradecenedioic acid and/or (z5)1,18-octadecenedioic acid acid) and only trace amounts of native ω-7 unsaturated α/ω-dicarboxylic acids (e.g., (z7)1,16-hexadecenedioic acid).

ω-5 unsaturated α/ω-dicarboxylic acids can be chemically converted to macrocyclic ketones similar to musk ketone fragrances (see e.g., International Patent Application Publication WO 2015/157719 A9). For example, using decarboxylative chemistry (z5)1,16-hexadecenedioic acid can be converted to the 15-membered macrocyclic ketones (5z)-cyclopentadec-5-en-1-one or (4z)-cyclopentadec-4-en-1-one. The latter is also known as exaltenone. Using non-decarboxylative chemistry (z5)1,16-hexadecenedioic acid can be converted to the 16-membered macrocyclic ketones (6Z)-cyclohexadec-6-en-1-one or (5Z)-cyclohexadec-5-en-1-one. The latter is also known as ambrettone or velvione.

### Example 10: Production of ω-5 Unsaturated Fatty Alcohols by Recombinant E. coli Strains Expressing an Acyl-ACP Reductase

Recombinant ΔFabA E. coli strain expressing a Δ9-tetradecanoyl-ACP desaturase, an acyl-ACP reductase and an alcohol dehydrogenase produces predominantly non-native ω-5 unsaturated fatty aldehydes and fatty alcohols (e.g., z11-hexadecenal and z11-hexadecenol) and only trace amounts of ω-7 unsaturated fatty aldehydes and fatty alcohols (e.g., z9-hexadecenal and z9-hexadecenol).

Strain sAS.571 (as prepared in Example 3) is transformed with a plasmid similar to pKM.023 (as prepared in Example 4), in which the FatA thioesterase gene is replaced by the AAR acyl-ACP reductase gene from Synechococcus elongatus (SEQ ID NOs: 34 and 35) (UniProtKB-Q54765) and optinonally the alrA alcohol dehydrogenase gene from Acinetobacter baylyi (SEQ ID NOs: 19 and 20) (UniProtKB-Q6F6R9).

The resulting strain is subjected to small scale fermentation and product analysis as described in Example 1. The recombinant proteobacterium produces predominantly non-native ω-5 unsaturated fatty aldehydes and fatty alcohols such as z9-tetradecenal, z11-hexadecenal, z13-tetradecenal, z9-tetradecenol, z11-hexadecenol and z13-tetradecenol and only trace amounts of ω-7 unsaturated fatty aldehydes and fatty alcohols (e.g., z9-hexadecenal and z9-hexadecenol).

### Example 11: Production of ω-5 Unsaturated Fatty Alcohol Acetate Esters by Recombinant E. coli Strains Expressing an Acyl-ACP Reductase

Recombinant ΔFabA E. coli strain expressing a Δ9-tetradecanoyl-ACP desaturase, an acyl-ACP reductase, an alcohol dehydrogenase and an acetyl-CoA transferase produces predominantly non-native ω-5 unsaturated fatty alcohol acetates (e.g., z11-hexadecenyl acetate) and only trace amounts of ω-7 unsaturated fatty alcohol acetates (e.g., z9-hexadecenyl acetate).

A gene coding for an acetyl-CoA transferase [EC 2.3.1.84], e.g., Atf1 from Saccharomyces cerevisiae (SEQ ID NOs: 23 and 24) (UniProtKB-Q6XBT3), under the control of an inducible or constitutive promoter, is integrated into the chromosome of strain sAS.571 (as prepared in Example 3). The resulting strain is transformed with the AAR acyl-ACP reductase containing plasmid prepared in Example 10.

The resulting strain is subjected to small scale fermentation and product analysis as described in Example 1. The recombinant proteobacterium produces non-native ω-5-unsaturated fatty alcohol acetates such as z11-hexadecenyl acetate, z9-tetradecenyl acetate and/or z13-octadecenyl acetate and only trace amounts of native ω7-unsaturated fatty alcohol acetates (e.g., z9-hexadecenyl acetate).

### Example 12: Production of Non-Native ω5-Unsaturated z9-Tetradecenoic Acid by a Recombinant E. coli Strain

This example shows that a recombinant ΔFabA E. coli strain expressing a Δ9-tetradecanoyl-ACP desaturase and a thioesterase from Clostridium thermocellum (UniProtKB-A3DJY9) predominantly produces non-native ω-5 unsaturated z9-tetradecenoic acid and only trace amounts of native ω-7 unsaturated z7-tetradecenoic acid.

The chromosomal copy of the fabB gene of strain sAS.571 (prepared as in Example 3) was replaced with a variant of fabB yielding strain ECKh-16885. The strain is transformed with plasmid pG_21306 (SC101 replicon, spectinomycin resistance marker) containing a thioesterase gene from Clostridium thermocellum under control of an inducible Ptrc promoter. The resulting strain was L29426.

Strain L29426 was subjected to small scale fermentation and product analysis as described in Example 1. As shown in Table 10, strain L29426 produced 285 mg/L of free fatty acids at 32° C. with a high content of C14 fatty acids (68%).

As shown in Table 10, 100% of the unsaturated fatty acid produced by L29426 had the double bond in the non-native ω-5 position with the majority being z9-tetradecenoic acid and smaller amounts of z11-hexadecenoic acid. The peak identification for z9-tetradecenoic acid and z11-hexadecenoic acid was performed by comparing their retention time and ion fragmentation pattern in GC/MS to that of authentic standards.

This example demonstrates that a recombinant ΔFabA E. coli strain expressing a Δ9-tetradecanoyl-ACP desaturase when co-expressed with an acyl-ACP thioesterase with high specificity for acyl-ACPs with C14 chain length such as from Clostridium thermocellum TE A3DJY9 produces predominantly non-native ω-5 unsaturated C14 fatty acids with only trace amounts of native ω-7 unsaturated C14 fatty acids.

TABLE 10
Composition and titer of free fatty acids (FFA) of recombinant E. coli strains

| | Total FFA | % C14 | % UFA | % ω5 | z7-C14:1 | z9-C14:1 | z9-C16:1 | z11-C16:1 | z11-C18:1 |
|---|---|---|---|---|---|---|---|---|---|
| strain | (mg/L) | FFA | of FFA | of UFA | (mg/L) | (mg/L) | (mg/L) | (mg/L) | (mg/L) |
| L29426 | 285 | 68 | 45 | 100 | 0 | 107 | 0 | 23 | 0 |

### Example 13: Production of ω-5 Unsaturated z9-Tertadecenol by Recombinant E. coli Strains

This example shows that a recombinant ΔFabA E. coli strain expressing a Δ9-tetradecanoyl-ACP desaturase, a thioesterase from Clostridium thermocellum (UniProtKB-A3DJY9) and a carboxylic acid reductase predominantly produces ω-5 unsaturated z9-tetradecenol) and only trace amounts of ω-7 unsaturated z7-tetradecenol).

The chromosomal copy of the fabB gene of strain sAS.619 (prepared as in Example 5) is replaced with a variant of fabB. The resulting strain is transformed with a plasmid (SC101 replicon, spectinomycin resistance marker) containing a thioesterase gene from Clostridium thermocellum under control of an inducible Ptrc promoter and containing a second operon, under control of an inducible PT5 promoter, with the carB carboxylic acid reductase gene from Mycobacterium smegmatis (SEQ ID NOs: 17 and 18) (UniProtKB-A0A653FCI4) and the alrA alcohol dehydrogenase gene from Acinetobacter baylyi (SEQ ID NOs: 19 and 20) (UniProtKB-Q6F6R9).

The strain is subjected to small scale fermentation and product analysis as described in Example 1. The strain is expected to produce predominantly C14 fatty acid derivatives. The strain is expected to produce predominantly ω-5 unsaturated z9-tetradecenol and only trace amounts of ω-7 unsaturated z7-tetradecenol.

### Example 14: Production of ω-5 Unsaturated Fatty Alcohols by Recombinant E. coli Strains with Attenuated FabA

This example shows that a recombinant E. coli strain with attenuated FabA expressing a Δ9-tetradecanoyl-ACP desaturase, a thioesterase, and a carboxylic acid reductase produces mixtures of ω-5 unsaturated fatty alcohols (e.g., z9-tetradecenol, z11-hexadecenol and z13-octadecenol) and ω-7 unsaturated fatty alcohols (e.g., z7-tetradecenol, z9-hexadecenol and z11-octadecenol).

The strain is a derivative of E. coli MG1655 and its genome is engineered as follows: the acyl-CoA dehydrogenase (fadE) gene is deleted and a variant of the transcriptional regulator FadR is overexpressed. Both modifications are optional. An operon controlled by a constitutive PT5 promoter is integrated into the chromosome that contained the Δ9-tetradecanoyl-ACP desaturase gene (without the plastid targeting leader sequence) from P. xhortorum (DES_Pxho), and flavodoxin reductase (fpr) (SEQ ID NOs: 13 and 14) (UniProtKB-P28861) and ferredoxin (petF) from Nostoc punctiforme PCC73102 (SEQ ID NOs: 5 and 6). Additionally, the chromosomal entD gene is deregulated by replacing the native iron-regulated promoter of entD with a constitutive PT5 promoter. Optionally, the gene for a 3-hydroxy-acyl-ACP dehydratase (FabZ) (SEQ ID NO: 15 and 16) (UniProtKB-A0A0M1I0X8) from Acinetobacter baylyi controlled by a constitutive PT5 promoter is also integrated.

The dual hydroxy-acyl-ACP dehydratase/isomerase fabA gene is attenuated as follows: the native promoter or RBS controlling FabA's transcription or translation initiation are modified or replaced with a weaker synthetic promoter or RBS, or FabA's start codon is altered from ATG to GTG or TTG or the native fabA gene is replaced with a fabA gene encoding an altered FabA protein variant which is less active or less stable, for example FabA^{ts} (see Johnson and Greenberg 1975, J. Bacteriol. 122: 570-574), or a combination thereof.

The resulting recombinant strain is transformed with plasmid pSVEN.250 or pSVEN.251 (see Example 5) or a similar expression plasmid containing the fatA thioesterase gene from Arabidopsis thaliana, the FabB β-ketoacyl-ACP synthase gene, a carB carboxylic acid reductase gene from Mycobacterium smegmatis (SEQ ID NOs: 17 and 18) (UniProtKB-A0A653FCI4) and the alrA alcohol dehydrogenase gene from Acinetobacter baylyi (SEQ ID NOs: 19 and 20) (UniProtKB-Q6F6R9)

The strain is subjected to small scale fermentation and product analysis as described in Example 1. The ω-5 and ω-7 unsaturated fatty alcohols, e.g., z9-tetradecenol, z11-hexadecenol, z13-octadecenol, z7-tetradecenol, z9-hexadecenol and z11-octadecenol are measured. Depending on the level of FabA attenuation, the strain may produce more ω-5 unsaturated than ω-7 unsaturated fatty alcohols, e.g., 90% of ω-5 unsaturated and 10% ω-7 unsaturated fatty alcohols (e.g. 80% of z11-hexadecenol and 10% of z13-octadecenol and 10% of z9-hexadecenol).

All references, including publications, patent applications, and patents, cited herein are hereby incorporated by reference to the same extent as if each reference were individually and specifically indicated to be incorporated by reference and were set forth in its entirety herein.

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Sequence Table

| SEQ ID NO: | Description | Sequence |
|---|---|---|
| 1 | myristyl-ACP | ATGGCTTCTACCTCTATCTCTAAAGTTAACCACATCCGTAA |
| | desaturase | AGTTGGTGTTACCGGTGTTATGGCTCCGCAGAAAATCGAA |
| | [Pelargonium x | ATCTTCAAATCTATGGAAGAATGGGGTAAACACAACATCC |
| | hortorum], | TGCCGCTGGCTAAACCGGTTGAAAAATCTTGGCAGCCGAC |
| | DNA sequence | CGACTTCCTGCCGGACCCGTCTTCTGAAGGTTTCATGGAAG |
| | without plastid | AATACAACGCTTTCAAAGAACGTACCCGTGAACTGCCGGA |
| | leader sequence | CGAATACTTCGTTGTTCTGGCTGGTGACATGATCACCGAAG |
| | | AAGCTCTGCCGACCTACCAGACCCTGGTTAACCGTCCGGAC |
| | | GAAGTTGCTGACGAAACCGGTCACTCTGAATCTCCGTGGG |
| | | CTGTTTGGTCTCGTGCTTGGACCGCTGAAGAAAACCGTCAC |
| | | GGTGACCTGCTGAACAAATACCTGTACCTGTCTGGTAAACT |
| | | GGACATGCGTCAGGTTGAAAAAACCATCCAGTACCTGATC |
| | | GCTCTGGGTCAGGACATCGGTACCGAAAAAAACCCGTACC |
| | | ACCTGTTCATCTACACCTCTTTCCAGGAACGTGCTACCTTC |
| | | ATCTCTCACGCTAACACCGCTAAACTGGCTCAGCAGCACG |
| | | GTGACAAACAGCTGGCTCAGATCTGCGGTACCATCGCTGCT |
| | | GACGAAAAACGTCACGAAACCGCTTACACCCGTATCGTTG |
| | | ACAAACTGTTCGAACTGGACCCGGACGAAACCATGTCTTG |
| | | CCTGGCTCACATGATGAAACGTAAAATCACCATGCCGGCT |
| | | CACCTGATGCGTGACGGTCGTGACCCGCACCTGTTCCAGCA |
| | | CTTCTCTGTgGTTGCTTCTCGTACCGGTGTTTACACCGTTAT |
| | | GGACTACATCAACATCCTGGAACACTTCGTTGAAAAATGG |
| | | AACATCGAAAAAATCACCGCTGGTCTGTCTGACAAAGGTC |
| | | GTGAAGCTCAGGACTACGTTTGCAAACTGGGTGAACGTCT |
| | | GCGTAAAGTTGAAGAACGTGCTCACCAGCGTGTTGTTCAG |
| | | GCTGACCCGATCCCGTTCTCTTGGATCTTCGACCGTAAAGT |
| | | TTAA |
| 2 | myristyl-ACP | MASTSISKVNHIRKVGVTGVMAPQKIEIFKSMEEWGKHNILPL |
| | desaturase | AKPVEKSWQPTDFLPDPSSEGFMEEYNAFKERTRELPDEYFV |
| | [Pelargonium x | VLAGDMITEEALPTYQTLVNRPDEVADETGHSESPWAVWSR |
| | hortorum], | AWTAEENRHGDLLNKYLYLSGKLDMRQVEKTIQYLIALGQDI |
| | mature protein | GTEKNPYHLFIYTSFQERATFISHANTAKLAQQHGDKQLAQIC |
| | | GTIAADEKRHETAYTRIVDKLFELDPDETMSCLAHMMKRKIT |
| | | MPAHLMRDGRDPHLFQHFSVVASRTGVYTVMDYINILEHFVE |
| | | KWNIEKITAGLSDKGREAQDYVCKLGERLRKVEERAHQRVV |
| | | QADPIPFSWIFDRKV |
| 3 | Ferredoxin | ATGACTCAAGCGAAAGCCAAAAAAGACCACGGTGACGTTC |
| | reductase PetH, | CTGTTAACACTTACCGTCCCAATGCTCCATTTATTGGCAAG |
| | Nostoc | GTAATATCTAATGAACCATTAGTCAAAGAAGGTGGTATTG |
| | punctiforme | GTATTGTTCAACACCTTAAATTTGACCTATCTGGTGGGGAT |
| | PCC73102, | TTGAAGTATATAGAAGGTCAAAGTATTGGCATTATTCCGCC |
| | DNA Sequence | AGGTTTAGACAAGAACGGCAAGCCTGAAAAACTCAGACTA |
| | | TATTCCATCGCCTCAACTCGTCATGGTGATGATGTAGATGA |
| | | TAAGACAGTATCACTGTGCGTCCGCCAGTTGGAGTACAAG |
| | | CACCCAGAAACTGGCGAAACAGTCTACGGTGTTTGCTCTAC |
| | | GCACCTGTGTTTCCTCAAGCCAGGGGAAGAGGTAAAAATT |
| | | ACAGGGCCTGTGGGTAAGGAAATGTTGTTACCCAATGACC |
| | | CTGATGCTAATGTTATCATGATGGCTACTGGAACAGGTATT |
| | | GCGCCGATGCGGGCTTACTTGTGGCGTCAGTTTAAAGATGC |
| | | GGAAAGAGCGGCTAACCCAGAATACCAATTTAAAGGATTC |
| | | TCTTGGCTAATATTTGGCGTACCTACAACTCCAAACCTTTT |
| | | ATATAAGGAAGAACTGGAAGAGATTCAACAAAAATATCCT |
| | | GAGAACTTCCGCCTAACTGCTGCCATCAGCCGCGAACAGA |
| | | AAAATCCCCAAGGCGGTAGAATGTATATTCAAGACCGCGT |
| | | AGCAGAACATGCTGATGAATTGTGGCAGTTGATTAAAAAT |
| | | GAAAAAACCCACACTTACATTTGCGGTTTGCGCGGTATGG |
| | | AAGAAGGTATTGATGCAGCCTTAACTGCTGCTGCTGCTAAG |
| | | GAAGGCGTAACCTGGAGTGATTACCAGAAGCAACTCAAGA |
| | | AAGCCGGTCGCTGGCACGTAGAAACTTACTAA |
| 4 | Ferredoxin | MTQAKAKKDHGDVPVNTYRPNAPFIGKVISNEPLVKEGGIGI |
| | reductase PetH, | VQHLKFDLSGGDLKYIEGQSIGIIPPGLDKNGKPEKLRLYSIAS |
| | Nostoc | TRHGDDVDDKTVSLCVRQLEYKHPETGETVYGVCSTHLCFL |
| | punctiforme | KPGEEVKITGPVGKEMLLPNDPDANVIMMATGTGIAPMRAYL |
| | PCC73102, | WRQFKDAERAANPEYQFKGFSWLIFGVPTTPNLLYKEELEEIQ |
| | mature protein | QKYPENFRLTAAISREQKNPQGGRMYIQDRVAEHADELWQLI |
| | | KNEKTHTYICGLRGMEEGIDAALTAAAAKEGVTWSDYQKQL |
| | | KKAGRWHVETY |
| 5 | Ferredoxin | ATGCCAACTTATAAAGTGACACTAATTAACGAGGCTGAAG |
| | PetF, Nostoc | GGCTGAACACAACCCTTGATGTTGAGGACGATACCTATATT |
| | punctiforme | CTAGACGCAGCTGAAGAAGCTGGTATTGACCTGCCCTACTC |
| | PCC73102, | TTGCCGCGCTGGTGCTTGCTCTACTTGTGCAGGTAAACTCG |
| | DNA sequence | TATCAGGTACCGTCGATCAAGGCGATCAATCATTCTTAGAT |
| | | GACGATCAAATAGAAGCTGGATATGTACTGACCTGTGTTG |
| | | CTTACCCAACTTCTAATGTCACGATCGAAACTCACAAAGAA |
| | | GAAGAACTCTATTAA |
| 6 | Ferredoxin PetF | MPTYKVTLINEAEGLNTTLDVEDDTYILDAAEEAGIDLPYSCR |
| | protein, Nostoc | AGACSTCAGKLVSGTVDQGDQSFLDDDQIEAGYVLTCVAYP |
| | punctiforme | TSNVTIETHKEEELY |
| | PCC73102 | |
| 7 | FatA | ATGGCTGTTGTATCTGCTGATCAAGGTAGTGTGGTTCAAGG |
| | thioesterase | TTTGGCTACTCTCGCGGATCAGCTCCGATTAGGTAGTTTGA |
| | from | CTGAAGATGGTTTATCTTATAAAGAGAAGTTTGTTGTTAGA |
| | Arabidopsos | TCTTACGAAGTGGGTAGTAACAAAACCGCTACTGTTGAAA |
| | thaliana, DNA | CCATTGCTAATCTTTTACAGGAGGTGGGATGTAATCATGCA |
| | sequence | CAAAGTGTTGGTTTTTCGACTGATGGGTTTGCAACAACAAC |
| | without plastid | TACTATGAGGAAGTTGCATCTCATTTGGGTTACTGCGAGAA |
| | leader sequence | TGCATATCGAGATCTATAAGTACCCTGCTTGGGGTGATGTG |
| | | GTTGAGATAGAGACTTGGTGTCAGAGTGAAGGAAGGATTG |
| | | GGACAAGGCGTGATTGGATTCTTAAGGATTCTGTCACTGGT |
| | | GAAGTCACTGGCCGTGCTACAAGCAAGTGGGTGATGATGA |
| | | ACCAAGACACGAGACGGCTTCAGAAAGTTTCTGATGATGT |
| | | TCGGGACGAGTACTTGGTCTTCTGTCCTCAAGAACCGAGGT |
| | | TAGCATTTCCGGAAGAGAATAACCGTAGCTTGAAGAAAAT |
| | | CCCGAAACTCGAAGATCCGGCTCAGTATTCAATGATTGGG |
| | | CTTAAGCCTAGACGAGCTGATCTCGACATGAACCAGCATG |
| | | TCAATAATGTCACCTATATTGGATGGGTTCTGGAGAGCATA |
| | | CCACAAGAAATTGTAGACACGCACGAGCTTCAGGTCATAA |
| | | CTCTGGATTATAGAAGAGAATGTCAACAAGACGATGTGGT |
| | | GGATTCACTCACCACCACCACCTCTGAAATTGGTGGAACCA |
| | | ATGGCTCTGCCACGTCTGGCACACAGGGCCACAACGATAG |
| | | CCAGTTCTTGCACCTCCTGAGGTTGTCTGGAGATGGTCAGG |
| | | AGATCAACCGCGGGACAACTCTGTGGAGAAAGAAGCCTTC |
| | | AAGTTAA |
| 8 | FatA | MAVVSADQGSVVQGLATLADQLRLGSLTEDGLSYKEKFVVR |
| | thioesterase | SYEVGSNKTATVETIANLLQEVGCNHAQSVGFSTDGFATTTT |
| | from | MRKLHLIWVTARMHIEIYKYPAWGDVVEIETWCQSEGRIGTR |
| | Arabidopsos | RDWILKDSVTGEVTGRATSKWVMMNQDTRRLQKVSDDVRD |
| | thaliana, | EYLVFCPQEPRLAFPEENNRSLKKIPKLEDPAQYSMIGLKPRR |
| | UniProtKB- | ADLDMNQHVNNVTYIGWVLESIPQEIVDTHELQVITLDYRRE |
| | Q42561 | CQQDDVVDSLTTTTSEIGGTNGSATSGTQGHNDSQFLHLLRLS |
| | (Genbank | GDGQEINRGTTLWRKKPSS |
| | NP_189147), | |
| | mature protein | |
| 9 | ABN54268.1 | ATGCAGAAGAAAAGATTTTCAAAGAAGTATGAAGTACATT |
| | Clostridium | ACTACGAAATCAACTCAATGCAGGAAGCAACTCTTCTCTCC |
| | thermocellum | CTGCTAAACTATATGGAGGACTGCGCAATATCCCACTCAAC |
| | ATCC 27405 | CTCTGCCGGATACGGTGTCAACGAGTTATTGGCTGCTGACG |
| | acyl-ACP | CAGGATGGGTATTATACCGCTGGTTAATTAAAATAGACAG |
| | thioesterase | ACTTCCCAAGCTCGGAGAAACAATTACTGTTCAGACATGG |
| | gene | GCCTCTTCCTTCGAACGCTTCTACGGCAACAGGGAATTTAT |
| | | CGTATTGGACGGCAGGGATAACCCCATTGTCAAAGCCTCA |
| | | TCCGTATGGATATATTTCAATATTAAAAAAAGAAAACCTAT |
| | | GAGAATCCCCCTCGAAATGGGAGATGCTTATGGCATAGAC |
| | | GAAACAAGAGCTTTGGAAGAACCCTTTACCGACTTCGATTT |
| | | TGATTTTGAACCCAAAGTTATTGAAGAATTTACTGTAAAAA |
| | | GAAGTGATATAGACACAAACAGCCACGTAAACAACAAGA |
| | | AATACGTTGACTGGATTATGGAAACCGTACCCCAGCAAAT |
| | | ATATGACAACTACAAAGTTACATCTCTTCAGATTATATACA |
| | | AAAAGGAATCTTCTTTGGGTTCAGGCATAAAGGCCGGATG |
| | | TGTAATTGATGAGCAAAATACCGATAATCCGCGGCTCCTTC |
| | | ACAAAATATGGGACAAGAATACCGGTTTGGAGCTTGTATC |
| | | CGCCGAAACAATCTGGCAAAAGATTCAGTCATAA |
| 10 | A3DJY9_HUN | MQKKRFSKKYEVHYYEINSMQEATLLSLLNYMEDCAISHSTS |
| | T2 Acyl-ACP | AGYGVNELLAADAGWVLYRWLIKIDRLPKLGETITVQTWAS |
| | thioesterase, | SFERFYGNREFIVLDGRDNPIVKASSVWIYFNIKKRKPMRIPLE |
| | Hungateiclostridium | MGDAYGIDETRALEEPFTDFDFDFEPKVIEEFTVKRSDIDTNSH |
| | thermocellum | VNNKKYVDWIMETVPQQIYDNYKVTSLQIIYKKESSLGSGIK |
| | | AGCVIDEQNTDNPRLLHKIWDKNTGLE |
| 11 | CCB49392.1 | ATGGCTTCCATGCACCGCTCTGTGTCCAGGGAGATTAAGAA |
| | Vitis vinifera | TACAAAGAAGACTTCTAGCTCTCCTCGCAAGGTGCAAGTA |
| | (wine grape) | ACCCATTCAATGCCACCACACAAGATTGAGATTTTCAAATC |
| | novel gene | CATGGAGAATTGGGTTGAGGAGAACGTTTTAATTCACCTG |
| | | AAGCCAGTTGAGAAATGTTGGCAACCTCAGGATTTTCTGCC |
| | | TCATCCTGCTTCTGATGGATTTCATGAGCGAGTCGAGGAGC |
| | | TAAAGGAGAGAGCAAAGGGGATCCCGGATGACTACTTTGT |
| | | CGTTTTGGTTGGAGATATGATCACTGAAGAAGCCCTTCCAA |
| | | CTTACCAAACACTGTTCAATACCACTGATGGAATCCGTGAT |
| | | GAAACAGGTGCAAGCCCCACTTCTTGGGCAACTTGGACAA |
| | | GGGCATGGACCGCTGAAGAGAACAGGCACGGTGACCTTCT |
| | | TAATAAGTATCTCTATCTGTCTGGAAGAGTAGACATGAAAC |
| | | AAATTGAGAAGACGATCCAGTATTTGATTAGGGCTGGAAT |
| | | GGATTTCCAGACGGAAAACAATCCGTACCTTTTATTCATCT |
| | | ATACTTCATTTCAAGAAAGGGCAACCTTCATATCCCATGGC |
| | | AATACTGCCAGGCTCGCCAAGCAACATGGGGACAAGAGCT |
| | | TGGCTCAAATATGTGGCATAATAGCCTCAGATGAGAAGCG |
| | | CCATGAAACTGCCTACACCAAGATAGTGGAAAAGCTCTTT |
| | | GAGATTGATCCCAATGGGACTGTCTTGGCTTTTGCAGACAG |
| | | GATGAGGAAGAAAATCACCATGCCGGCCCTCTTGATGTAT |
| | | GATGGATGTGATGACGACCTTTTTGAACACTTCTCAGCAGT |
| | | TGCTCAGCGGCTTGGTGTGTATACTGCCAAGGACTATGTTG |
| | | ATAACTTAGAATTCTTTGTGGAAAGATGGAATGTGGAAAA |
| | | GCTAACTGGGCTTTCTAGTGAGGGGCGAAAAGCTCAGGAT |
| | | TATGTTTGTGGGTTAGCTAAAAGATTGAGAACACTGGAGG |
| | | AGAGAGCTCAAGAAAAGGCTAAGCAAGCACCCACCATTCC |
| | | TTTCAGTTGGATTTTTGATAGAGAAGTGAAGCTCTGA |
| 12 | F6HB23_VITV | MASMHRSVSREIKNTKKTSSSPRKVQVTHSMPPHKIEIFKSME |
| | I Novel protein | NWVEENVLIHLKPVEKCWQPQDFLPHPASDGFHER VEELKER |
| | (Vitis vinifera) | AKGIPDDYFVVLVGDMITEEALPTYQTLFNTTDGIRDETGASP |
| | | TSWATWTRAWTAEENRHGDLLNKYLYLSGRVDMKQIEKTIQ |
| | | YLIRAGMDFQTENNPYLLFIYTSFQERATFISHGNTARLAKQH |
| | | GDKSLAQICGIIASDEKRHETAYTKIVEKLFEIDPNGTVLAFAD |
| | | RMRKKITMPALLMYDGCDDDLFEHFSAVAQRLGVYTAKDY |
| | | VDNLEFFVERWNVEKLTGLSSEGRKAQDYVCGLAKRLRTLE |
| | | ERAQEKAKQAPTIPFSWIFDREV |
| 13 | Flavodoxin/ferr | ATGGCTGATTGGGTAACAGGCAAAGTCACTAAAGTGCAGA |
| | edoxin-NADP+ | ACTGGACCGACGCCCTGTTTAGTCTCACCGTTCACGCCCCC |
| | reductase, fpr, | GTGCTTCCGTTTACCGCCGGGCAATTTACCAAGCTTGGCCT |
| | Escherichia | TGAAATCGACGGCGAACGCGTCCAGCGCGCCTACTCCTAT |
| | coli, DNA | GTAAACTCGCCCGATAATCCCGATCTGGAGTTTTACCTGGT |
| | sequence | CACCGTCCCCGATGGCAAATTAAGCCCACGACTGGCGGCA |
| | | CTGAAACCAGGCGATGAAGTGCAGGTGGTTAGCGAAGCGG |
| | | CAGGATTCTTTGTGCTCGATGAAGTGCCGCACTGCGAAACG |
| | | CTATGGATGCTGGCAACCGGTACAGCGATTGGCCCTTATTT |
| | | ATCGATTCTGCAACTAGGTAAAGATTTAGATCGCTTCAAAA |
| | | ATCTGGTCCTGGTGCACGCCGCACGTTATGCCGCCGACTTA |
| | | AGCTATTTGCCACTGATGCAGGAACTGGAAAAACGCTACG |
| | | AAGGAAAACTGCGCATTCAGACGGTGGTCAGTCGGGAAAC |
| | | GGCAGCGGGGTCGCTCACCGGACGGATACCGGCATTAATT |
| | | GAAAGTGGGGAACTGGAAAGCACGATTGGCCTGCCGATGA |
| | | ATAAAGAAACCAGCCATGTGATGCTGTGCGGCAATCCACA |
| | | GATGGTGCGCGATACACAACAGTTGCTGAAAGAGACCCGG |
| | | CAGATGACGAAACATTTACGTCGCCGACCGGGCCATATGA |
| | | CAGCGGAGCATTACTGGTAA |
| 14 | Flavodoxin/ferr | MADWVTGKVTKVQNWTDALFSLTVHAPVLPFTAGQFTKLG |
| | edoxin-NADP+ | LEIDGERVQRAYSYVNSPDNPDLEFYLVTVPDGKLSPRLAAL |
| | reductase, Fpr, | KPGDEVQVVSEAAGFFVLDEVPHCETLWMLATGTAIGPYLSI |
| | Escherichia | LQLGKDLDRFKNLVLVHAARYAADLSYLPLMQELEKRYEGK |
| | coli, protein | LRIQTVVSRETAAGSLTGRIPALIESGELESTIGLPMNKETSHV |
| | | MLCGNPQMVRDTQQLLKETRQMTKHLRRRPGHMTAEHY |
| 15 | 3-hydroxy-acyl- | ATGATGACTGAGTCAAACACTCCTGCGTTTACGATCCCTGA |
| | [acyl-carrier- | ATTGCCAATGGACATTCAAAAAATTCGTGAATATTTGCCAC |
| | protein] | ATCGCTATCCATTCCTATTGGTTGATCGTGTAGTTGAAGTT |
| | dehydratase, | GGTGAAAATAATATTGTTGGCTATAAAAACGTTTCGATTAA |
| | FabZ, | CGAAGAGTTTTTTCAGGGCCATTTTCCTGATTATCCGATTA |
| | Acinetobacter | TGCCAGGTGTCTTGATTGTAGAGGCTTTGGCACAAATTTCT |
| | baylyi, DNA | GGTATTTTGGGTTTTATTATGAATAATGAAACTCCTAAACC |
| | sequence | AGGTTCTTTGTTTCTGTTCGCAGGGGCTGAGAAAGTTCGTT |
| | | TCAAAAAACAAGTGGTTGCTGGTGATCAGCTCGTTTTAAAA |
| | | GCTGAACTTGTCATGCAAAAACGTGGTATCTACAAATACA |
| | | ATTGTACTGCTACGGTTGATGGTAAAGTCGCTACAACCGCT |
| | | GAAATTATAGTTTCGCATTTAAGAACAGAGCAGGCATG |
| 16 | 3-hydroxy-acyl- | MMTESNTPAFTIPELPMDIQKIREYLPHRYPFLLVDRVVEVGE |
| | [acyl-carrier- | NNIVGYKNVSINEEFFQGHFPDYPIMPGVLIVEALAQISGILGFI |
| | protein] | MNNETPKPGSLFLFAGAEKVRFKKQVVAGDQLVLKAELVMQ |
| | dehydratase, | KRGIYKYNCTATVDGKVATTAEIIVSHLRTEQA |
| | FabZ, | |
| | Acinetobacter | |
| | baylyi, protein | |
| 17 | Carboxylic | ATGGGCACGAGCGATGTTCACGACGCGACCGACGGCGTTA |
| | Acid Reductase, | CCGAGACTGCACTGGATGATGAGCAGAGCACTCGTCGTAT |
| | CarB, | TGCAGAACTGTACGCAACGGACCCAGAGTTCGCAGCAGCA |
| | Mycobacterium | GCTCCTCTGCCGGCCGTTGTCGATGCGGCGCACAAACCGG |
| | smegmatis, | GCCTGCGTCTGGCGGAAATCCTGCAGACCCTGTTCACCGGC |
| | DNA sequence | TACGGCGATCGTCCGGCGCTGGGCTATCGTGCACGTGAGCT |
| | | GGCGACGGACGAAGGCGGTCGTACGGTCACGCGTCTGCTG |
| | | CCGCGCTTCGATACCCTGACCTATGCACAGGTGTGGAGCCG |
| | | TGTTCAAGCAGTGGCTGCAGCGTTGCGTCACAATTTCGCAC |
| | | AACCGATTTACCCGGGCGACGCGGTCGCGACTATCGGCTTT |
| | | GCGAGCCCGGACTATTTGACGCTGGATCTGGTGTGCGCGTA |
| | | TCTGGGCCTGGTCAGCGTTCCTTTGCAGCATAACGCTCCGG |
| | | TGTCTCGCCTGGCCCCGATTCTGGCCGAGGTGGAACCGCGT |
| | | ATTCTGACGGTGAGCGCAGAATACCTGGACCTGGCGGTTG |
| | | AATCCGTCCGTGATGTGAACTCCGTCAGCCAGCTGGTTGTT |
| | | TTCGACCATCATCCGGAAGTGGACGATCACCGTGACGCAC |
| | | TGGCTCGCGCACGCGAGCAGCTGGCCGGCAAAGGTATCGC |
| | | AGTTACGACCCTGGATGCGATCGCAGACGAAGGCGCAGGT |
| | | TTGCCGGCTGAGCCGATTTACACGGCGGATCACGATCAGC |
| | | GTCTGGCCATGATTCTGTATACCAGCGGCTCTACGGGTGCT |
| | | CCGAAAGGCGCGATGTACACCGAAGCGATGGTGGCTCGCC |
| | | TGTGGACTATGAGCTTTATCACGGGCGACCCGACCCCGGTT |
| | | ATCAACGTGAACTTCATGCCGCTGAACCATCTGGGCGGTCG |
| | | TATCCCGATTAGCACCGCCGTGCAGAATGGCGGTACCAGC |
| | | TACTTCGTTCCGGAAAGCGACATGAGCACGCTGTTTGAGG |
| | | ATCTGGCCCTGGTCCGCCCTACCGAACTGGGTCTGGTGCCG |
| | | CGTGTTGCGGACATGCTGTACCAGCATCATCTGGCGACCGT |
| | | GGATCGCCTGGTGACCCAGGGCGCGGACGAACTGACTGCG |
| | | GAAAAGCAGGCCGGTGCGGAACTGCGTGAACAGGTCTTGG |
| | | GCGGTCGTGTTATCACCGGTTTTGTTTCCACCGCGCCGTTG |
| | | GCGGCAGAGATGCGTGCTTTTCTGGATATCACCTTGGGTGC |
| | | ACACATCGTTGACGGTTACGGTCTGACCGAAACCGGTGCG |
| | | GTCACCCGTGATGGTGTGATTGTTCGTCCTCCGGTCATTGA |
| | | TTACAAGCTGATCGATGTGCCGGAGCTGGGTTACTTCTCCA |
| | | CCGACAAACCGTACCCGCGTGGCGAGCTGCTGGTTCGTAG |
| | | CCAAACGTTGACTCCGGGTTACTACAAGCGCCCAGAAGTC |
| | | ACCGCGTCCGTTTTCGATCGCGACGGCTATTACCACACCGG |
| | | CGACGTGATGGCAGAAACCGCGCCAGACCACCTGGTGTAT |
| | | GTGGACCGCCGCAACAATGTTCTGAAGCTGGCGCAAGGTG |
| | | AATTTGTCGCCGTGGCTAACCTGGAGGCCGTTTTCAGCGGC |
| | | GCTGCTCTGGTCCGCCAGATTTTCGTGTATGGTAACAGCGA |
| | | GCGCAGCTTTCTGTTGGCTGTTGTTGTCCCTACCCCGGAGG |
| | | CGCTGGAGCAATACGACCCTGCCGCATTGAAAGCAGCCCT |
| | | GGCGGATTCGCTGCAGCGTACGGCGCGTGATGCCGAGCTG |
| | | CAGAGCTATGAAGTGCCGGCGGACTTCATTGTTGAGACTG |
| | | AGCCTTTTAGCGCTGCGAACGGTCTGCTGAGCGGTGTTGGC |
| | | AAGTTGCTGCGTCCGAATTTGAAGGATCGCTACGGTCAGC |
| | | GTTTGGAGCAGATGTACGCGGACATCGCGGCTACGCAGGC |
| | | GAACCAATTGCGTGAACTGCGCCGTGCTGCGGCTACTCAA |
| | | CCGGTGATCGACACGCTGACGCAAGCTGCGGCGACCATCC |
| | | TGGGTACCGGCAGCGAGGTTGCAAGCGACGCACACTTTAC |
| | | TGATTTGGGCGGTGATTCTCTGAGCGCGCTGACGTTGAGCA |
| | | ACTTGCTGTCTGACTTCTTTGGCTTTGAAGTCCCGGTTGGC |
| | | ACGATTGTTAACCCAGCGACTAATCTGGCACAGCTGGCGC |
| | | AACATATCGAGGCGCAGCGCACGGCGGGTGACCGCCGTCC |
| | | ATCCTTTACGACGGTCCACGGTGCGGATGCTACGGAAATCC |
| | | GTGCAAGCGAACTGACTCTGGACAAATTCATCGACGCTGA |
| | | GACTCTGCGCGCAGCACCTGGTTTGCCGAAGGTTACGACTG |
| | | AGCCGCGTACGGTCCTGTTGAGCGGTGCCAATGGTTGGTTG |
| | | GGCCGCTTCCTGACCCTGCAGTGGCTGGAACGTTTGGCACC |
| | | GGTTGGCGGTACCCTGATCACCATTGTGCGCGGTCGTGACG |
| | | ATGCAGCGGCACGTGCACGTTTGACTCAGGCTTACGATAC |
| | | GGACCCAGAGCTGTCCCGCCGCTTCGCTGAGTTGGCGGATC |
| | | GCCACTTGCGTGTGGTGGCAGGTGATATCGGCGATCCGAA |
| | | TCTGGGCCTGACCCCGGAGATTTGGCACCGTCTGGCAGCA |
| | | GAGGTCGATCTGGTCGTTCATCCAGCGGCCCTGGTCAACCA |
| | | CGTCCTGCCGTACCGCCAGCTGTTTGGTCCGAATGTTGTTG |
| | | GCACCGCCGAAGTTATCAAGTTGGCTCTGACCGAGCGCAT |
| | | CAAGCCTGTTACCTACCTGTCCACGGTTAGCGTCGCGATGG |
| | | GTATTCCTGATTTTGAGGAGGACGGTGACATTCGTACCGTC |
| | | AGCCCGGTTCGTCCGCTGGATGGTGGCTATGCAAATGGCTA |
| | | TGGCAACAGCAAGTGGGCTGGCGAGGTGCTGCTGCGCGAG |
| | | GCACATGACCTGTGTGGCCTGCCGGTTGCGACGTTTCGTAG |
| | | CGACATGATTCTGGCCCACCCGCGCTACCGTGGCCAAGTG |
| | | AATGTGCCGGACATGTTCACCCGTCTGCTGCTGTCCCTGCT |
| | | GATCACGGGTGTGGCACCGCGTTCCTTCTACATTGGTGATG |
| | | GCGAGCGTCCGCGTGCACACTACCCGGGCCTGACCGTCGA |
| | | TTTTGTTGCGGAAGCGGTTACTACCCTGGGTGCTCAGCAAC |
| | | GTGAGGGTTATGTCTCGTATGACGTTATGAATCCGCACGAT |
| | | GACGGTATTAGCTTGGATGTCTTTGTGGACTGGCTGATTCG |
| | | TGCGGGCCACCCAATTGACCGTGTTGACGACTATGATGACT |
| | | GGGTGCGTCGTTTTGAAACCGCGTTGACCGCCTTGCCGGAG |
| | | AAACGTCGTGCGCAGACCGTTCTGCCGCTGCTGCATGCCTT |
| | | TCGCGCGCCACAGGCGCCGTTGCGTGGCGCCCCTGAACCG |
| | | ACCGAAGTGTTTCATGCAGCGGTGCGTACCGCTAAAGTCG |
| | | GTCCGGGTGATATTCCGCACCTGGATGAAGCCCTGATCGAC |
| | | AAGTACATCCGTGACCTGCGCGAGTTCGGTCTGATTTAG |
| 18 | Carboxylic | MGTSDVHDATDGVTETALDDEQSTRRIAELYATDPEFAAAAP |
| | Acid Reductase, | LPAVVDAAHKPGLRLAEILQTLFTGYGDRPALGYRARELATD |
| | CarB, | EGGRTVTRLLPRFDTLTYAQVWSRVQAVAAALRHNFAQPIYP |
| | Mycobacterium | GDAVATIGFASPDYLTLDLVCAYLGLVSVPLQHNAPVSRLAPI |
| | smegmatis, | LAEVEPRILTVSAEYLDLAVESVRDVNSVSQLVVFDHHPEVD |
| | protein | DHRDALARAREQLAGKGIAVTTLDAIADEGAGLPAEPIYTAD |
| | | HDQRLAMILYTSGSTGAPKGAMYTEAMVARLWTMSFITGDP |
| | | TPVINVNFMPLNHLGGRIPISTAVQNGGTSYFVPESDMSTLFE |
| | | DLALVRPTELGLVPRVADMLYQHHLATVDRLVTQGADELTA |
| | | EKQAGAELREQVLGGRVITGFVSTAPLAAEMRAFLDITLGAHI |
| | | VDGYGLTETGAVTRDGVIVRPPVIDYKLIDVPELGYFSTDKPY |
| | | PRGELLVRSQTLTPGYYKRPEVTASVFDRDGYYHTGDVMAE |
| | | TAPDHLVYVDRRNNVLKLAQGEFVAVANLEAVESGAALVRQ |
| | | IFVYGNSERSFLLAVVVPTPEALEQYDPAALKAALADSLQRT |
| | | ARDAELQSYEVPADFIVETEPFSAANGLLSGVGKLLRPNLKDR |
| | | YGQRLEQMYADIAATQANQLRELRRAAATQPVIDTLTQAAA |
| | | TILGTGSEVASDAHFTDLGGDSLSALTLSNLLSDFFGFEVPVG |
| | | TIVNPATNLAQLAQHIEAQRTAGDRRPSFTTVHGADATEIRAS |
| | | ELTLDKFIDAETLRAAPGLPKVTTEPRTVLLSGANGWLGRFLT |
| | | LQWLERLAPVGGTLITIVRGRDDAAARARLTQAYDTDPELSR |
| | | RFAELADRHLRVVAGDIGDPNLGLTPEIWHRLAAEVDLVVHP |
| | | AALVNHVLPYRQLFGPNVVGTAEVIKLALTERIKPVTYLSTVS |
| | | VAMGIPDFEEDGDIRTVSPVRPLDGGYANGYGNSKWAGEVL |
| | | LREAHDLCGLPVATFRSDMILAHPRYRGQVNVPDMFTRLLLS |
| | | LLITGVAPRSFYIGDGERPRAHYPGLTVDFVAEAVTTLGAQQR |
| | | EGYVSYDVMNPHDDGISLDVFVDWLIRAGHPIDRVDDYDDW |
| | | VRRFETALTALPEKRRAQTVLPLLHAFRAPQAPLRGAPEPTEV |
| | | FHAAVRTAKVGPGDIPHLDEALIDKYIRDLREFGLI* |
| 19 | Alcohol | ATGGCAACAACTAATGTGATTCATGCTTATGCTGCAATGCA |
| | dehydrogenase, | GGCAGGTGAAGCACTCGTGCCTTATTCGTTTGATGCAGGCG |
| | AlrA, | AACTGCAACCACATCAGGTTGAAGTTAAAGTCGAATATTG |
| | Acinetobacter | TGGGCTGTGCCATTCCGATGTCTCGGTACTCAACAACGAAT |
| | baylyi, DNA | GGCATTCTTCGGTTTATCCAGTCGTGGCAGGTCATGAAGTG |
| | sequence | ATTGGTACGATTACCCAACTGGGAAGTGAAGCCAAAGGAC |
| | | TAAAAATTGGTCAACGTGTTGGTATTGGCTGGACGGCAGA |
| | | AAGCTGTCAGGCCTGTGACCAATGCATCAGTGGTCAGCAG |
| | | GTATTGTGCACGGGCGAAAATACCGCAACTATTATTGGTCA |
| | | TGCTGGTGGCTTTGCAGATAAGGTTCGTGCAGGCTGGCAAT |
| | | GGGTCATTCCCCTGCCCGACGAACTCGATCCGACCAGTGCT |
| | | GGTCCTTTGCTGTGTGGCGGAATCACAGTATTTGATCCAAT |
| | | TTTAAAACATCAGATTCAGGCTATTCATCATGTTGCTGTGA |
| | | TTGGTATCGGTGGTTTGGGACATATGGCCATCAAGCTACTT |
| | | AAAGCATGGGGCTGTGAAATTACTGCGTTTAGTTCAAATCC |
| | | AAACAAAACCGATGAGCTCAAAGCTATGGGGGCCGATCAC |
| | | GTGGTCAATAGCCGTGATGATGCCGAAATTAAATCGCAAC |
| | | AGGGTAAATTTGATTTACTGCTGAGTACAGTTAATGTGCCT |
| | | TTAAACTGGAATGCGTATCTAAACACACTGGCACCCAATG |
| | | GCACTTTCCATTTTTTGGGCGTGGTGATGGAACCAATCCCT |
| | | GTACCTGTCGGTGCGCTGCTAGGAGGTGCCAAATCGCTAA |
| | | CAGCATCACCAACTGGCTCGCCTGCTGCCTTACGTAAGCTG |
| | | CTCGAATTTGCGGCACGTAAGAATATCGCACCTCAAATCG |
| | | AGATGTATCCTATGTCGGAGCTGAATGAGGCCATCGAACG |
| | | CTTACATTCGGGTCAAGCACGTTATCGGATTGTACTTAAAG |
| | | CCGATTTTTAA |
| 20 | Alcohol | MATTNVIHAYAAMQAGEALVPYSFDAGELQPHQVEVKVEYC |
| | dehydrogenase, | GLCHSDVSVLNNEWHSSVYPVVAGHEVIGTITQLGSEAKGLK |
| | AlrA, | IGQRVGIGWTAESCQACDQCISGQQVLCTGENTATIIGHAGGF |
| | Acinetobacter | ADKVRAGWQWVIPLPDELDPTSAGPLLCGGITVFDPILKHQIQ |
| | baylyi, protein | AIHHVAVIGIGGLGHMAIKLLKAWGCEITAFSSNPNKTDELKA |
| | | MGADHVVNSRDDAEIKSQQGKFDLLLSTVNVPLNWNAYLNT |
| | | LAPNGTFHFLGVVMEPIPVPVGALLGGAKSLTASPTGSPAALR |
| | | KLLEFAARKNIAPQIEMYPMSELNEAIERLHSGQARYRIVLKA |
| | | DF |
| 21 | Hybrid fusion | ATGCCAACACTGCCCAGAACATTTGACGACATTCAGTCCCG |
| | cytochrome | ACTGATTAACGCCACCTCCAGGGTGGTGCCGATGCAGAGG |
| | P450 | CAAATTCAGGGACTGAAATTCTTAATGAGCGCCAAGAGGA |
| | Cyp153A16(G3 | AGACCTTCGGCCCACGCCGACCGATGCCCGAATTCGTTGA |
| | 07A)-RedRhF | AACACCCATCCCGGACGTTAACACGCTGGCCCTTGAGGAC |
| | Fusion Protein, | ATCGATGTCAGCAATCCGTTTTTATACCGGCAGGGTCAGTG |
| | DNA Sequence | GCGCGCCTATTTCAAACGGTTGCGTGATGAGGCGCCGGTCC |
| | | ATTACCAGAAGAACAGCCCTTTCGGCCCCTTCTGGTCGGTA |
| | | ACTCGGTTTGAAGACATCCTGTTCGTGGATAAGAGTCACGA |
| | | CCTGTTTTCCGCCGAGCCGCAAATCATTCTCGGTGACCCTC |
| | | CGGAGGGGCTGTCGGTGGAAATGTTCATAGCGATGGATCC |
| | | GCCGAAACACGATGTGCAGCGCAGCTCGGTGCAGGGAGTA |
| | | GTGGCACCGAAAAACCTGAAGGAGATGGAGGGGCTGATCC |
| | | GATCACGCACCGGCGATGTGCTTGACAGCCTGCCTACAGA |
| | | CAAACCCTTTAACTGGGTACCTGCTGTTTCCAAGGAACTCA |
| | | CAGGCCGCATGCTGGCGACGCTTCTGGATTTTCCTTACGAG |
| | | GAACGCCACAAGCTGGTTGAGTGGTCGGACAGAATGGCAG |
| | | GTGCAGCATCGGCCACCGGCGGGGAGTTTGCCGATGAAAA |
| | | TGCCATGTTTGACGACGCGGCAGACATGGCCCGGTCTTTCT |
| | | CCAGGCTTTGGCGGGACAAGGAGGCGCGCCGCGCAGCAGG |
| | | CGAGGAGCCCGGTTTCGATTTGATCAGCCTGTTGCAGAGCA |
| | | ACAAAGAAACGAAAGACCTGATCAATCGGCCGATGGAGTT |
| | | TATCGGTAATTTGACGCTGCTCATAGTCGcCGGCAACGATA |
| | | CGACGCGCAACTCGATGAGTGGTGGCCTGGTGGCCATGAA |
| | | CGAATTCCCCAGGGAATTTGAAAAATTGAAGGCAAAACCG |
| | | GAGTTGATTCCGAACATGGTGTCGGAAATCATCCGCTGGC |
| | | AAACGCCGCTGGCCTATATGCGCCGAATCGCCAAGCAGGA |
| | | TGTCGAACTGGGCGGCCAGACCATCAAGAAGGGTGATCGA |
| | | GTTGTCATGTGGTACGCGTCGGGTAACCGGGACGAGCGCA |
| | | AATTTGACAACCCCGATCAGTTCATCATTGATCGCAAGGAC |
| | | GCACGAAACCACATGTCGTTCGGCTATGGGGTTCACCGTTG |
| | | CATGGGCAACCGTCTGGCTGAACTGCAACTGCGCATCCTCT |
| | | GGGAAGAAATACTCAAGCGTTTTGACAACATCGAAGTCGT |
| | | CGAAGAGCCCGAGCGGGTGCAGTCCAACTTCGTGCGGGGC |
| | | TATTCCAGGTTGATGGTCAAACTGACACCGAACAGTGTACT |
| | | CCATCGTCATCAACCTGTCACCATCGGCGAGCCGGCCGCTC |
| | | GTGCTGTGAGCCGCACGGTGACCGTTGAGCGTCTTGATCGC |
| | | ATTGCCGACGATGTCCTTCGCCTGGTCCTTCGCGATGCTGG |
| | | AGGTAAAACCCTCCCGACGTGGACGCCTGGCGCTCACATC |
| | | GACCTGGATCTGGGtGCTCTGAGCCGTCAGTATTCGCTCTG |
| | | CGGCGCTCCGGATGCTCCGTCGTACGAAATCGCCGTGCACT |
| | | TAGATCCgGAAAGCCGTGGTGGAAGCCGCTATATTCATGAA |
| | | CAGCTGGAAGTTGGAAGTCCGCTGCGTATGCGtGGCCCACG |
| | | CAACCATTTCGCCCTGGATCCGGGtGCGGAACATTACGTGT |
| | | TTGTTGCCGGGGGTATCGGCATCACGCCGGTGCTGGCAATG |
| | | GCGGATCATGCCCGTGCGCGTGGTTGGTCGTACGAACTGC |
| | | ATTATTGTGGTCGTAATCGTAGCGGTATGGCTTACCTGGAA |
| | | CGCGTCGCGGGACATGGTGACCGCGCtGCCTTGCACGTATC |
| | | TGAAGAAGGCACCCGCATTGATCTGGCGGCATTACTTGCTG |
| | | AACCGGCGCCGGGCGTGCAAATCTACGCCTGCGGTCCGGG |
| | | CCGTTTATTAGCGGGTCTTGAAGACGCGTCTCGTAATTGGC |
| | | CGGATGGCGCGCTTCATGTGGAGCATTTCACTTCGAGTTTA |
| | | GCCGCTTTGGATCCGGATGTCGAACATGCCTTTGATTTGGA |
| | | GCTGCGTGACTCTGGCCTTACCGTTCGCGTCGAGCCAACTC |
| | | AGACCGTTTTAGACGCTTTGCGTGCGAACAATATCGACGTC |
| | | CCGTCGGATTGCGAAGAGGGGCTGTGTGGTTCTTGCGAAG |
| | | TAGCCGTTCTGGATGGCGAGGTTGATCACCGTGATACCGTT |
| | | CTGACTAAGGCCGAGCGCGCCGCGAATCGTCAGATGATGA |
| | | CTTGCTGCAGTCGTGCATGCGGTGATCGTCTGGCGCTGCGC |
| | | CTCTAA |
| 22 | Hybrid fusion | MPTLPRTFDDIQSRLINATSRVVPMQRQIQGLKFLMSAKRKTF |
| | cytochrome | GPRRPMPEFVETPIPDVNTLALEDIDVSNPFLYRQGQWRAYFK |
| | P450 | RLRDEAPVHYQKNSPFGPFWSVTRFEDILFVDKSHDLFSAEPQ |
| | Cyp153A16(G3 | IILGDPPEGLSVEMFIAMDPPKHDVQRSSVQGVVAPKNLKEM |
| | 07A)-RedRhF | EGLIRSRTGDVLDSLPTDKPFNWVPAVSKELTGRMLATLLDFP |
| | Fusion Protein | YEERHKLVEWSDRMAGAASATGGEFADENAMEDDAADMA |
| | Sequence | RSFSRLWRDKEARRAAGEEPGFDLISLLQSNKETKDLINRPME |
| | | FIGNLTLLIVAGNDTTRNSMSGGLVAMNEFPREFEKLKAKPEL |
| | | IPNMVSEIIRWQTPLAYMRRIAKQDVELGGQTIKKGDRVVMW |
| | | YASGNRDERKFDNPDQFIIDRKDARNHMSFGYGVHRCMGNR |
| | | LAELQLRILWEEILKRFDNIEVVEEPERVQSNFVRGYSRLMVK |
| | | LTPNSVLHRHQPVTIGEPAARAVSRTVTVERLDRIADDVLRLV |
| | | LRDAGGKTLPTWTPGAHIDLDLGALSRQYSLCGAPDAPSYEI |
| | | AVHLDPESRGGSRYIHEQLEVGSPLRMRGPRNHFALDPGAEH |
| | | YVFVAGGIGITPVLAMADHARARGWSYELHYCGRNRSGMA |
| | | YLERVAGHGDRAALHVSEEGTRIDLAALLAEPAPGVQIYACG |
| | | PGRLLAGLEDASRNWPDGALHVEHFTSSLAALDPDVEHAFDL |
| | | ELRDSGLTVRVEPTQTVLDALRANNIDVPSDCEEGLCGSCEV |
| | | AVLDGEVDHRDTVLTKAERAANRQMMTCCSRACGDRLALRL |
| 23 | Alcohol acetyl- | ATGAACGAAATCGACGAAAAAAACCAGGCTCCGGTTCACC |
| | CoA transferase | AGGAATGCCTGAAAGAAATGATCCAGAACGGTCACGCTCG |
| | Atf1, | TCGTATGGGTTCTGTTGAAGACCTGTACGTTGCTCTGAACC |
| | Saccharomyces | GTCAGAACCTGTACCGTAACTTCTGCACCTACGGTGAACTG |
| | cerevisiae, | TCTGACTACTGCACCCGTGACCAGCTGACCCTGGCTCTGCG |
| | DNA Sequence | TGAAATCTGCCTGAAAAACCCGACCCTGCTGCACATCGTTC |
| | | TGCCGACCCGTTGGCCGAACCACGAAAACTACTACCGTTCT |
| | | TCTGAATACTACTCTCGTCCGCACCCGGTTCACGACTACAT |
| | | CTCTGTTCTGCAGGAACTGAAACTGTCTGGTGTTGTTCTGA |
| | | ACGAACAGCCGGAATACTCTGCTGTTATGAAACAGATCCT |
| | | GGAAGAATTCAAAAACTCTAAAGGTTCTTACACCGCTAAA |
| | | ATCTTCAAACTGACCACCACCCTGACCATCCCGTACTTCGG |
| | | TCCGACCGGTCCGTCTTGGCGTCTGATCTGCCTGCCGGAAG |
| | | AACACACCGAAAAATGGAAAAAATTCATCTTCGTTTCTAA |
| | | CCACTGCATGTCTGACGGTCGTTCTTCTATCCACTTCTTCCA |
| | | CGACCTGCGTGACGAACTGAACAACATCAAAACCCCGCCG |
| | | AAAAAACTGGACTACATCTTCAAATACGAAGAAGACTACC |
| | | AGCTGCTGCGTAAACTGCCGGAACCGATCGAAAAAGTTAT |
| | | CGACTTCCGTCCGCCGTACCTGTTCATCCCGAAATCTCTGC |
| | | TGTCTGGTTTCATCTACAACCACCTGCGTTTCTCTTCTAAAG |
| | | GTGTTTGCATGCGTATGGACGACGTTGAAAAAACCGACGA |
| | | CGTTGTTACCGAAATCATCAACATCTCTCCGACCGAATTCC |
| | | AGGCTATCAAAGCTAACATCAAATCTAACATCCAGGGTAA |
| | | ATGCACCATCACCCCGTTCCTGCACGTTTGCTGGTTCGTTTC |
| | | TCTGCACAAATGGGGTAAATTCTTCAAACCGCTGAACTTCG |
| | | AATGGCTGACCGACATCTTCATCCCGGCTGACTGCCGTTCT |
| | | CAGCTGCCGGACGACGACGAAATGCGTCAGATGTACCGTT |
| | | ACGGTGCTAACGTTGGTTTCATCGACTTCACCCCGTGGATC |
| | | TCTGAATTCGACATGAACGACAACAAAGAAAACTTCTGGC |
| | | CGCTGATCGAACACTACCACGAAGTTATCTCTGAAGCTCTG |
| | | CGTAACAAAAAACACCTGCACGGTCTGGGTTTCAACATCC |
| | | AGGGTTTCGTTCAGAAATACGTTAACATCGACAAAGTTATG |
| | | TGCGACCGTGCTATCGGTAAACGTCGTGGTGGTACCCTGCT |
| | | GTCTAACGTTGGTCTGTTCAACCAGCTGGAAGAACCGGAC |
| | | GCTAAATACTCTATCTGCGACCTGGCTTTCGGTCAGTTCCA |
| | | GGGTTCTTGGCACCAGGCTTTCTCTCTGGGTGTTTGCTCTAC |
| | | CAACGTTAAAGGTATGAACATCGTTGTTGCTTCTACCAAAA |
| | | ACGTTGTTGGTTCTCAGGAATCTCTGGAAGAACTGTGCTCT |
| | | ATCTACAAAGCTCTGCTGCTGGGTCCGTAG |
| 24 | Alcohol acetyl- | MNEIDEKNQAPVHQECLKEMIQNGHARRMGSVEDLYVALNR |
| | CoA transferase | QNLYRNFCTYGELSDYCTRDQLTLALREICLKNPTLLHIVLPT |
| | Atf1, | RWPNHENYYRSSEYYSRPHPVHDYISVLQELKLSGVVLNEQP |
| | Saccharomyces | EYSAVMKQILEEFKNSKGSYTAKIFKLTTTLTIPYFGPTGPSWR |
| | cerevisiae, | LICLPEEHTEKWKKFIFVSNHCMSDGRSSIHFFHDLRDELNNIK |
| | protein | TPPKKLDYIFKYEEDYQLLRKLPEPIEKVIDFRPPYLFIPKSLLS |
| | sequence | GFIYNHLRFSSKGVCMRMDDVEKTDDVVTEIINISPTEFQAIK |
| | | ANIKSNIQGKCTITPFLHVCWFVSLHKWGKFFKPLNFEWLTDI |
| | | FIPADCRSQLPDDDEMRQMYRYGANVGFIDFTPWISEFDMND |
| | | NKENFWPLIEHYHEVISEALRNKKHLHGLGFNIQGFVQKYVNI |
| | | DKVMCDRAIGKRRGGTLLSNVGLFNQLEEPDAKYSICDLAFG |
| | | QFQGSWHQAFSLGVCSTNVKGMNIVVASTKNVVGSQESLEE |
| | | LCSIYKALLLGP |
| 25 | Phosphopantetheinyl | ATGAAAACTACGCATACCTCCCTCCCCTTTGCCGGACATAC |
| | transferase | GCTGCATTTTGTTGAGTTCGATCCGGCGAATTTTTGTGAGC |
| | EntD, DNA | AGGATTTACTCTGGCTGCCGCACTACGCACAACTGCAACAC |
| | Sequence | GCTGGACGTAAACGTAAAACAGAGCATTTAGCCGGACGGA |
| | | TCGCTGCTGTTTATGCTTTGCGGGAATATGGCTATAAATGT |
| | | GTGCCCGCAATCGGCGAGCTACGCCAACCTGTCTGGCCTGC |
| | | GGAGGTATACGGCAGTATTAGCCACTGTGGGACTACGGCA |
| | | TTAGCCGTGGTATCTCGTCAACCGATTGGCATTGATATAGA |
| | | AGAAATTTTTTCTGTACAAACCGCAAGAGAATTGACAGAC |
| | | AACATTATTACACCAGCGGAACACGAGCGACTCGCAGACT |
| | | GCGGTTTAGCCTTTTCTCTGGCGCTGACACTGGCATTTTCC |
| | | GCCAAAGAGAGCGCATTTAAGGCAAGTGAGATCCAAACTG |
| | | ATGCAGGTTTTCTGGACTATCAGATAATTAGCTGGAATAAA |
| | | CAGCAGGTCATCATTCATCGTGAGAATGAGATGTTTGCTGT |
| | | GCACTGGCAGATAAAAGAAAAGATAGTCATAACGCTGTGC |
| | | CAACACGATTAA |
| 26 | Phosphopantetheinyl | MKTTHTSLPFAGHTLHFVEFDPANFCEQDLLWLPHYAQLQH |
| | transferase | AGRKRKTEHLAGRIAAVYALREYGYKCVPAIGELRQPVWPA |
| | EntD, protein | EVYGSISHCGTTALAVVSRQPIGIDIEEIFSVQTARELTDNIITPA |
| | | EHERLADCGLAFSLALTLAFSAKESAFKASEIQTDAGFLDYQII |
| | | SWNKQQVIIHRENEMFAVHWQIKEKIVITLCQHD |
| 27 | FabZ from | MSLSIEQIMEIIPHRYPMLLVDRVEEIEPGKRAVGYKNVTFNE |
| | Clostridium | QIFQGHYPGKPIMPGVLMIEALAQLGGVAILSLDKYKGKKPIL |
| | acetobutylicum, | GAVKNAKFRRMVVPGDVLKLEIEIVKVKGPAGIGKGIATVNG |
| | protein | EKAVEAEITFMIV |
| 28 | FabZ from | MTVNPDAPALPTLPLAVETIQGLLPHRYPFALVDRIIDYVPGE |
| | Synechococcus | RAVGIKNVTFNEPQFQGHFPGRPLMPGVLIVEAMAQVGGVIV |
| | elongatus, | TLMPDMPQGLFVFAGIDQVRFRRPVVPGDQLVLSAQLLSVKR |
| | protein | RRFCKIQGEAMVDGQLAASGELLESLVE |
| 29 | Alcohol | MYDYIIVGAGSAGCVLANRLSADPSKRVCLLEAGPRDTNPLI |
| | dehydrogenase | HMPLGIALLSNSKKLNWAFQTAPQQNLNGRSLFWPRGKTLG |
| | from | GSSSINAMVYIRGHEDDYHAWEQAAGRYWGWYRALELFKR |
| | Pseudomonas | LECNQRFDKSEHHGVDGELAVSDLKYINPLSKAFVQAGMEA |
| | oleovorans, | NINFNGDFNGEYQDGVGFYQVTQKNGQRWSSARAFLHGVLS |
| | protein | RPNLDIITDAHASKILFEDRKAVGVSYIKKNMHHQVKTTSGGE |
| | | VLLSLGAVGTPHLLMLSGVGAAAELKEHGVSLVHDLPEVGK |
| | | NLQDHLDITLMCAANSREPIGVALSFIPRGVSGLFSYVFKREG |
| | | FLTSNVAESGGFVKSSPDRDRPNLQFHFLPTYLKDHGRKIAGG |
| | | YGYTLHICDLLPKSRGRIGLKSANPLQPPLIDPNYLSDHEDIKT |
| | | MIAGIKIGRAILQAPSMAKHFKHEVVPGQAVKTDDEIIEDIRRR |
| | | AETIYHPVGTCRMGKDPASVVDPCLKIRGLANIRVVDASIMP |
| | | HLVAGNTNAPTIMIAENAAEIIMRNLDVEALEASAEFAREGAE |
| | | LELAMIAVCM |
| 30 | Acetaldehyde | MRYIDPNQPGSKVQFKAQYENFIGGQWVPPVKGEYFGNSSPV |
| | dehydrogenase | DGKVFTQIPRSSVEDIELALDAAHKAKADWNKASPTVRSNVL |
| | 2 from | LKIADRLEENLELLAVAETWENGKPIRETLAADIPLAIDHFRYF |
| | Acinetobacter | AGCIRAQEGGISEIDEDTIAYHFHEPLGVVGQIIPWNFPILMAA |
| | baylyi, protein | WKLAPALAAGNCIVLKPAEQTPSSILVLAELIQDLLPPGVLNIV |
| | | NGYGAEVGRPLATNPRISKIAFTGSTKVGQMIMQYATENIIPV |
| | | TLELGGKSPNIFFEDILDKEDDYLEKTLEGFAMFALNQGEVCT |
| | | CPSRALVQESIADKFLEMAVERVKRIKTGHPLDTETMIGAQAS |
| | | KQQFDKILGCIDTGRNEGAQLLTGGDARHDVDGGFYIEPTIFK |
| | | GNNSMKIFQEEIFGPVLSVTTFKDFDDAMRIANDTIYGLGAGV |
| | | WSRSAHTSYRAGRAIEAGRVWTNCYHLYPAHAAFGGYKQSG |
| | | IGRENHRMMLDHYQQTKNLLVSYSTKPMGFF |
| 31 | Acetaldehyde | MIYAQPGQEGSVVSFKSRYENYIGGEWVAPVKGQYFDNITPV |
| | dehydrogenase | TGAVFCEVPRSTAEDIDLALDAAHKAAPAWGKTSPTERSNILL |
| | from | KIADRIEANLEKLAVAETWDNGKAVRETLNADVPLAADHLR |
| | Marinobacter | YFAGCIRAQEGHMSEIDHNTVAYHFHEPLGVVGQIIPWNFPLL |
| | aquaeoli, | MAAWKLGPCLASGNCTVLKPAEQTPASILVLMDIIGDLLPPG |
| | protein | VINIVNGYGIEAGQALATSKRIAKIAFTGSTPVGSHILKCAAEN |
| | | IIPSTVELGGKSPNIYFSDVMKAEPEFVDKCVEGLVLAFFNQG |
| | | EICTCPSRALVQEDMFEEFMQKVVERTKSIKRGNPLDTDVQV |
| | | GAQASKEQFDKIMSYMEIGRQEGAVVLTGGDREHLEGEFNN |
| | | GFYIQPTLFKGDNKMRVFQEEIFGPVVGVTTFKTEEEALAIAN |
| | | DTEFGLGAGVWTRDTNLAYRMGRNIQAGRVWMNCYHAYP |
| | | AHAAFGGYKKSGIGRENHKMALEHYQQTKCMLTSYDTNPLG |
| | | FF |
| 32 | Acetaldehyde | MTIPISLAKLNSSADTHSALEVFNLQKVASSARRGKFGIAERIA |
| | dehydrogenase | ALNLLKETIQRREPEIIAALAADFRKPASEVKLTEIFPVLQEINH |
| | from | AKRNLKDWMKPRRVRAALSVAGTRAGLRYEPKGVCLIIAPW |
| | Pseudomonas | NYPFNLSFGPLVSALAAGNSVVIKPSELTPHTATLIGSIVREAF |
| | oleovorans, | SVDLVAVVEGDAAVSQELLALPFDHIFFTGSPRVGKLVMEAA |
| | protein | SKTLASVTLELGGKSPTIIGPTANLPKAARNIVWGKFSNNGQT |
| | | CIAPDHVFVHRCIAQKFNEILVKEIVRVYGKDFAAQRRSADYC |
| | | RIVNDQHFNRINKLLTDAKAKGAKILQGGQVDATERLVVPTV |
| | | LSNVTAAMDINHEEIFGPLLPIIEYDDIDSVIKRVNDGDKPLAL |
| | | YVFSEDKQFVNNIVARTSSGSVGVNLSVVHFLHPNLPFGGVN |
| | | NSGIGSAHGVYGFRAFSHEKPVLIDKFSITHWLFPPYTKKVKQ |
| | | LIGITVKYLS |
| 33 | 3-oxoacyl-[acyl | MKRAVITGLGIVSSIGNNQQEVLASLREGRSGITFSQELKDSG |
| | carrier protein] | MRSHVWGNVKLDTTGLIDRKVVRFMSDASIYAFLSMEQAIA |
| | synthase 1, | DAGLSPEAYQNNPRVGLIAGSGGGSPRFQVFGADAMRGPRGL |
| | FabB, | KAVGPYVVTKAMASGVSACLATPFKIHGVNYSISSACATSAH |
| | Escherichia | CIGNAVEQIQLGKQDIVFAGGGEELCWEMACEFDAMGALST |
| | coli, protein | KYNDTPEKASRTYDAHRDGFVIAGGGGMVVVEELEHALARG |
| | | AHIYAEIVGYGATSDGADMVAPSGEGAVRCMKMAMHGVDT |
| | | PIDYLNSHGTSTPVGDVKELAAIREVFGDKSPAISATKAMTGH |
| | | SLGAAGVQEAIYSLLMLEHGFIAPSINIEELDEQAAGLNIVTET |
| | | TDRELTTVMSNSFGFGGTNATLVMRKLKD |
| 34 | Acyl-[acyl | ATGTTCGGTCTTATCGGTCATCTCACCAGTTTGGAGCAGGC |
| | carrier protein] | CCGCGACGTTTCTCGCAGGATGGGCTACGACGAATACGCC |
| | reductase, | GATCAAGGATTGGAGTTTTGGAGTAGCGCTCCTCCTCAAAT |
| | AAR, | CGTTGATGAAATCACAGTCACCAGTGCCACAGGCAAGGTG |
| | Synechococcus | ATTCACGGTCGCTACATCGAATCGTGTTTCTTGCCGGAAAT |
| | elongatus, DNA | GCTGGCGGCGCGCCGCTTCAAAACAGCCACGCGCAAAGTT |
| | sequence | CTCAATGCCATGTCCCATGCCCAAAAACACGGCATCGACA |
| | | TCTCGGCCTTGGGGGGCTTTACCTCGATTATTTTCGAGAAT |
| | | TTCGATTTGGCCAGTTTGCGGCAAGTGCGCGACACTACCTT |
| | | GGAGTTTGAACGGTTCACCACCGGCAATACTCACACGGCC |
| | | TACGTAATCTGTAGACAGGTGGAAGCCGCTGCTAAAACGC |
| | | TGGGCATCGACATTACCCAAGCGACAGTAGCGGTTGTCGG |
| | | CGCGACTGGCGATATCGGTAGCGCTGTCTGCCGCTGGCTCG |
| | | ACCTCAAACTGGGTGTCGGTGATTTGATCCTGACGGCGCGC |
| | | AATCAGGAGCGTTTGGATAACCTGCAGGCTGAACTCGGCC |
| | | GGGGCAAGATTCTGCCCTTGGAAGCCGCTCTGCCGGAAGC |
| | | TGACTTTATCGTGTGGGTCGCCAGTATGCCTCAGGGCGTAG |
| | | TGATCGACCCAGCAACCCTGAAGCAACCCTGCGTCCTAATC |
| | | GACGGGGGCTACCCCAAAAACTTGGGCAGCAAAGTCCAAG |
| | | GTGAGGGCATCTATGTCCTCAATGGCGGGGTAGTTGAACA |
| | | TTGCTTCGACATCGACTGGCAGATCATGTCCGCTGCAGAGA |
| | | TGGCGCGGCCCGAGCGCCAGATGTTTGCCTGCTTTGCCGAG |
| | | GCGATGCTCTTGGAATTTGAAGGCTGGCATACTAACTTCTC |
| | | CTGGGGCCGCAACCAAATCACGATCGAGAAGATGGAAGCG |
| | | ATCGGTGAGGCATCGGTGCGCCACGGCTTCCAACCCTTGGC |
| | | ATTGGCAATTTGA |
| 35 | Acyl-[acyl | MFGLIGHLTSLEQARDVSRRMGYDEYADQGLEFWSSAPPQIV |
| | carrier protein] | DEITVTSATGKVIHGRYIESCFLPEMLAARRFKTATRKVLNAM |
| | reductase, | SHAQKHGIDISALGGFTSIIFENFDLASLRQVRDTTLEFERFTTG |
| | AAR, | NTHTAYVICRQVEAAAKTLGIDITQATVAVVGATGDIGSAVC |
| | Synechococcus | RWLDLKLGVGDLILTARNQERLDNLQAELGRGKILPLEAALP |
| | elongatus, | EADFIVWVASMPQGVVIDPATLKQPCVLIDGGYPKNLGSKVQ |
| | protein | GEGIYVLNGGVVEHCFDIDWQIMSAAEMARPERQMFACFAE |
| | | AMLLEFEGWHTNFSWGRNQITIEKMEAIGEASVRHGFQPLALAI |

What is claimed is:

1. A recombinant proteobacterium comprising a heterologous acyl-ACP desaturase and a heterologous acyl-ACP thioesterase; wherein a native dual 3-hydroxy acyl-ACP dehydratase/isomerase (FabA) is deleted or attenuated; and wherein the recombinant proteobacterium produces a non-native monounsaturated free fatty acid or a derivative thereof.

2. The recombinant proteobacterium of claim 1, wherein the recombinant bacterium is a γ-proteobacterium.

3. The recombinant proteobacterium of claim 1 or 2, wherein the recombinant proteobacterium is selected from the group consisting of Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, and Marinobacter aquaeolei.

4. The recombinant proteobacterium of any one of the preceding claims, wherein the heterologous acyl-ACP-desaturase is Δ9-tetradecanoyl-acyl-ACP desaturase.

5. The recombinant proteobacterium of any one of the preceding claims, wherein the heterologous acyl-ACP desaturase has about 85% sequence identity to SEQ ID NO: 2 or SEQ ID NO: 12.

6. The recombinant proteobacteria of any one of the preceding claims, wherein the heterologous acyl-ACP thioesterase is a plant FatA-type thioesterase, a plant FatB-type thioesterase, or a bacterial acyl-ACP thioestersase.

7. The recombinant proteobacterium of any one of the preceding claims, wherein the heterologous acyl-ACP thioesterase has about 85% sequence identity to SEQ ID NO: 8.

8. The recombinant proteobacterium of any one of the preceding claims, wherein the non-native monounsaturated free fatty acid is a ω5-monounsaturated free fatty acid or a derivative thereof.

9. The recombinant proteobacterium of any one of the preceding claims, wherein an amount of non-native monounsaturated free fatty acids or derivatives thereof produced by the recombinant proteobacterium is greater than 90% of the total amount of native and non-native monounsaturated free fatty acids or derivatives thereof produced by the recombinant proteobacterium.

10. The recombinant proteobacterium of any one of the preceding claims, wherein the non-native monounsaturated free fatty acid is selected from the group consisting of z9-tetradecenoic acid, z11-hexadecenoic acid, z13-octadecenoic acid, and a combination thereof.

11. The recombinant proteobacterium of any one of the preceding claims, further comprising a heterologous ferredoxin.

12. The recombinant proteobacterium of any one of the preceding claims, wherein the recombinant bacterium overexpresses endogenous flavodoxin reductase or ferredoxin reductase or expresses heterologous ferredoxin reductase or heterologous flavodoxin reductase.

13. The recombinant proteobacterium of any one of the preceding claims, wherein the recombinant proteobacterium further comprises a heterologous 3-hydroxy acyl-ACP-dehydratase (FabZ).

14. The recombinant proteobacterium of any one of the preceding claims, wherein the recombinant proteobacterium further comprises (1) an increased level of expression of FadR polypeptide, and/or (2) an increased level of DNA binding activity, compared to a corresponding wild-type proteobacterium.

15. The recombinant proteobacterium of any one of the preceding claims, wherein the recombinant proteobacterium further comprises a heterologous carboxylic acid reductase.

16. The recombinant proteobacterium of claim 15, wherein the recombinant proteobacterium produces an unsaturated fatty aldehyde or an unsaturated fatty alcohol.

17. The recombinant proteobacterium of claim 16, wherein the unsaturated fatty aldehyde or unsaturated fatty alcohol is selected from the group consisting of z9-tetradecenal, z11-hexadecenal, z13-octadececanal, z9-tetradecenol, z11-hexadecenol, z13-octadececanol, and a combination thereof.

18. The recombinant proteobacterium of any one of claims 15-17, wherein the heterologous carboxylic acid reductase has about 85% sequence identity to SEQ ID NO: 18.

19. The recombinant proteobacterium of any one of the preceding claims, wherein the recombinant bacterium further comprises a heterologous alcohol dehydrogenase.

20. The recombinant proteobacterium of claim 19, wherein the heterologous alcohol dehydrogenase has about 85% sequence identity to SEQ ID NO: 20.

21. The recombinant proteobacterium of any one claim 15-20, wherein the recombinant bacterium further comprises a heterologous phosphopantetheinyl transferase.

22. The recombinant proteobacterium of claim 21, wherein the heterologous phosphopantetheinyl transferase has about 85% sequence identity to SEQ ID NO: 26.

23. The recombinant proteobacterium of any one of claims 15-22, wherein the recombinant proteobacterium further comprises a heterologous alcohol acetyl-CoA transferase.

24. The recombinant proteobacterium of claim 23, wherein the heterologous alcohol acetyl-CoA transferase has about 85% sequence identity to SEQ ID NO: 24.

25. The recombinant proteobacterium of claim 23 or 24, wherein the recombinant bacterium produces an unsaturated fatty alcohol acetate.

26. The recombinant proteobacterium of claim 25, wherein the unsaturated fatty alcohol acetate is selected from the group consisting of z9-tetradecenyl acetate, z11-hexadecenyl acetate, z13-octadecenyl acetate, and a combination thereof.

27. The recombinant proteobacterium of any one of the preceding claims, wherein the recombinant bacterium further comprises a heterologous ω-hydroxylase.

28. The recombinant proteobacterium of claim 27, wherein the heterologous ω-hydroxylase has about 85% sequence identity to SEQ ID NO: 22.

29. The recombinant proteobacterium of claim 27 or 28, wherein the recombinant proteobacterium produces an unsaturated ω-hydroxy fatty acid.

30. The recombinant proteobacterium of claim 29, wherein the unsaturated ω-hydroxy fatty acid is selected from the group consisting of (z9)14-hydroxy-tetradecenoic acid, (z11)16-hydroxy-hexadecenoic acid, (z13)18-hydroxy-octadecenoic acid, and a combination thereof.

31. The recombinant proteobacterium of claims 27-29, wherein the recombinant proteobacterium further comprises a heterologous alcohol oxidase/dehydrogenase and/or a heterologous aldehyde dehydrogenase.

32. The recombinant proteobacterium of claim 31, wherein the recombinant proteobacterium produces an unsaturated α/ω-dicarboxylic acid.

33. The recombinant proteobacterium of claim 32, wherein the heterologous α/ω-dicarboxylic acid is selected from the group consisting of (z5)1,14-tetradecenedioic acid, (z5)1,16-hexadecenedioic acid, (z5)1,18-octadecenedioic acid, and a combination thereof.

34. The recombinant proteobacterium of any one of the preceding claims, wherein the fatty acid derivative comprises a fatty alcohol, an ω-hydroxy fatty acid, a fatty alcohol acetate, a fatty aldehyde, or an α/ω-dicarboxylic acid.

35. The recombinant proteobacterium of claim 1, further comprising a heterologous 3-hydroxy acyl-ACP-dehydratase, a heterologous ferredoxin, and a heterologous flavodoxin reductase.

36. The recombinant proteobacterium of claim 1, further comprising a heterologous β-ketoacyl-ACP-synthase and/or the recombinant proteobacterium has an increased level of expression of endogenous β-ketoacyl-ACP-synthase compared to a corresponding wild-type proteobacterium.

37. The recombinant proteobacterium of claim 1, further comprising a heterologous carboxylic acid reductase, a heterologous alcohol dehydrogenase, a heterologous phosphopantetheinyl transferase, and a heterologous β-ketoacyl-ACP-synthase.

38. The recombinant proteobacterium of claim 1, further comprising a heterologous ω-hydroxylase, and a heterologous β-ketoacyl-ACP-synthase.

39. The recombinant proteobacterium of claim 1, further comprising a heterologous carboxylic acid reductase and a heterologous acetyl-CoA transferase.

40. The recombinant proteobacterium of claim 1, further comprising a carboxylic acid reductase and the recombinant proteobacterium has one or more deletions in endogenous alcohol dehydrogenase and/or endogenous aldehyde reductase genes.

41. The recombinant proteobacterium of claim 1, further comprising a heterologous alcohol dehydrogenase, a heterologous ω-hydroxylase, and a heterologous aldehyde dehydrogenase.

42. A method for producing a non-native monounsaturated free fatty acid or a derivative thereof, the method comprising culturing the recombinant proteobacterium of any one of claims 1 to 41.

43. The method of claim 41, further comprising isolating the non-native monounsaturated free fatty acid or derivative thereof.

44. A monounsaturated free fatty acid or derivative thereof prepared by the method of any one of claims 42 and 43.

45. The monounsaturated free fatty acid or derivative thereof of claim 44, wherein the monounsaturated free fatty acid or derivative thereof is purified.

46. The monounsaturated free fatty acid or derivative thereof of any one of claims 44 and 45, wherein the monounsaturated free fatty acid or derivative thereof is purified by a method selected from the group consisting of a two-step centrifugation and water-washing; decanting centrifugation and solvent extraction from a biomass; and whole broth extraction with a water immiscible solvent.

47. A product prepared from the monounsaturated free fatty acid or derivative thereof of any one of claims 44-46.

48. The product of claim 46, wherein the product is a pheromone fragrance or a nutritional supplement.

49. A method of making the product of any one of claims 47 and 48.

50. A nucleotide sequence encoding a Δ9-tetradecanoyl-acyl-ACP desaturase having at least 85% sequence identity to SEQ ID NO: 12.

51. The nucleotide sequence of claim 50, wherein the nucleotide sequence is operably linked to one or more heterologous regulatory elements.

52. A vector comprising a nucleotide sequence encoding a Δ9-tetradecanoyl-acyl-ACP desaturase having at least 85% sequence identity to SEQ ID NO: 12.

53. A recombinant host cell comprising a nucleotide sequence encoding a Δ9-tetradecanoyl-acyl-ACP desaturase having at least 85% sequence identity to SEQ ID NO: 12.

54. A composition comprising a monounsaturated free fatty acid or derivative thereof prepared by the method of culturing the recombinant proteobacterium of any one of claims 1 to 41.

55. The composition of claim 54, wherein the composition is a fermentation product of the recombinant proteobacterium.

56. The composition of any one of claim 54 or 55, wherein the composition is an insect pheromone or an insect pheromone precursor.

57. The composition of any one of claim 54 or 55, wherein the composition is a fragrance or a fragrance precursor.

58. The composition of any one of claims 54 to 57, wherein the monounsaturated free fatty acid or derivative thereof is purified.

59. The composition of claim 57, wherein the monounsaturated free fatty acid or derivative thereof is purified by a method selected from the group consisting of a two-step centrifugation and water-washing; decanting centrifugation and solvent extraction from a biomass; and whole broth extraction with a water immiscible solvent.

60. A method for preparing an insect pheromone or insect pheromone precursor comprising culturing a recombinant proteobacterium of any one of claims 1 to 41 and isolating the non-native monounsaturated free fatty acid or derivative thereof.

61. A method for preparing a fragrance or fragrance precursor comprising culturing a recombinant proteobacterium of any one of claims 1 to 41 and isolating the non-native monounsaturated free fatty acid or derivative thereof.

62. Use of the recombinant proteobacterium of any one of claims 1 to 41.

63. The use of claim 62, wherein the proteobacterium is used for producing (1) an insect pheromone or pheromone precursor or (2) a fragrance or fragrance precursor.

64. Use of the composition comprising a monounsaturated free fatty acid or derivative thereof prepared by culturing the recombinant proteobacterium of any one of claims 1 to 41.

65. The use of claim 64, wherein the composition is a fermentation product.

66. A recombinant proteobacterium comprising a heterologous acyl-ACP desaturase, a heterologous ferredoxin, a heterologous flavodoxin reductase or a heterologous ferredoxin reductase, a heterologous carboxylic acid reductase, and a heterologous 3-hydroxy-acyl-ACP dehydratase; wherein a native dual 3-hydroxy acyl-ACP dehydratase/isomerase (FabA) is deleted or attenuated; and wherein the recombinant proteobacterium produces a non-native monounsaturated free fatty acid or a derivative thereof.

67. A recombinant proteobacterium comprising a heterologous acyl-ACP desaturase, a heterologous ferredoxin, a heterologous flavodoxin reductase or a heterologous ferredoxin reductase, a heterologous acyl-ACP reductase and a heterologous alcohol dehydrogenase; wherein a native dual 3-hydroxy acyl-ACP dehydratase/isomerase (FabA) is deleted or attenuated; and wherein the recombinant proteobacterium produces a non-native monounsaturated fatty aldehyde and/or fatty alcohol.

68. The recombinant proteobacterium of claim 66 further comprising a heterologous acetyl-COA transferase.

69. A recombinant proteobacterium comprising a heterologous acyl-ACP desaturase, a heterologous ferredoxin, a heterologous flavodoxin reductase or a heterologous ferredoxin reductase, a heterologous ω-hydroxylase, and a heterologous 3-hydroxy-acyl-ACP dehydratase; wherein a native dual 3-hydroxy acyl-ACP dehydratase/isomerase (FabA) is deleted or attenuated; and wherein the recombinant proteobacterium produces a non-native monounsaturated ω-hydroxy fatty acid or a derivative thereof.

70. The recombinant proteobacterium of claim 69, further comprising a heterologous alcohol dehydrogenase and/or a heterologous aldehyde dehydrogenase.
